(19) United States
(12) Patent Application Publication
(10) Pub. No.: US 2023/0173141 A1
(43) Pub. Date: Jun. 8, 2023

(54) NANOFIBER-ENABLED ENCAPSULATION DEVICES AND USES THEREOF

(71) Applicant: CORNELL UNIVERSITY, Ithaca, NY (US)

(72) Inventors: Wanjun LIU, Ithaca, NY (US); Minglin MA, Ithaca, NY (US); James A. FLANDERS, Ithaca, NY (US); Longhai WANG, Ithaca, NY (US); Daniel T. BOWERS, Ithaca, NY (US); Qingsheng LIU, Ithaca, NY (US)

(21) Appl. No.: 17/916,215

(22) PCT Filed: Apr. 2, 2021
(86) PCT No.: PCT/US2021/025492

§ 371 Date: Sep. 30, 2022

Related U.S. Application Data
(60) Provisional application No. 63/004,331, filed on Apr. 2, 2020.

(51) Int. Cl. A61L 27/54; B82Y 5/00; A61L 27/52; A61L 27/56; A61L 27/34

(52) U.S. Cl. CPC A61L 27/54; B82Y 5/00; A61L 27/52; A61L 27/56; A61L 27/34; A61L 2400/12

(57) ABSTRACT

The present application relates to implantable therapeutic delivery system, its method of making and use. The therapeutic delivery system includes a nanofiber core substrate having proximal and distal ends, and an interior nanofiber wall defining an internal space extending longitudinally along the core substrate, with one or more therapeutic agents positioned within the internal space. A hydrogel surrounds the nanofiber core substrate, where the hydrogel includes 0.1% to 20% of an alginate mixture. The alginate mixture includes zwitterionically modified alginate and pure alginate in a ratio of 1:1000 to 1000:1 (v/v). Also disclosed is a thermo sealing device useful for the formation of the implantable therapeutic delivery system.

This application claims the priority benefit of U.S. Provisional Patent Application Ser. No. 63/004,331, filed Apr. 2, 2020, which is hereby incorporated by reference in its entirety.

This invention was made with government support under grant number 1R01DK105967-01A1 awarded by the National Institutes of Health. The government has certain rights in the invention.

### FIELD

The present disclosure relates to an implantable nanofiber-enabled therapeutic delivery system and methods of using the same.

### BACKGROUND

Type 1 diabetes (T1D) is an auto-immune disease characterized by a loss of β cells (Scharp and Marchetti, “Encapsulated Islets for Diabetes Therapy: History, Current Progress, and Critical Issues Requiring Solution,” Adv. Drug Deliver. Rev. 67-68:35-73 (2014); Desai and Shea, “Advances in Islet Encapsulation Technologies,” Nat. Rev. Drug Discov. 16:338-350 (2017); Veiseh et al., “Managing Diabetes with Nanomedicine: Challenges and Opportunities,” Nat. Rev. Drug Discov. 14:45-57 (2015); Katsarou et al., “Type 1 Diabetes Mellitus,” Nature Reviews Disease Primers 3:1-17 (2017)). Patients must frequently monitor their blood glucose levels and undergo insulin therapy to maintain blood glucose in a healthy range. This task is not only stressful but omnipresent for patients at every point of time in their lives. Current therapies use either injection of multiple insulin types or an insulin pump to suit a patient's needs (Ernst et al., “Nanotechnology in Cell Replacement Therapies for Type 1 Diabetes,” Adv Drug Deliver Rev 139:116-138 (2019); Bowers et al., “Engineering the Vasculature for Islet Transplantation,” Acta Biomater. 95:131-151 (2019); Yu et al., “Microneedle-array Patches Loaded with Hypoxia-sensitive Vesicles Provide Fast Glucose-responsive Insulin Delivery,” Proc. Natl. Acad Sci. USA 112:8260-8265 (2015)). Other devices such as Continuous Glucose Monitors (CGMs) offer more information and peace of mind to patients, but nonetheless require extensive input and effort from patients (Kovatchev et al., “Comparison of the Numerical and Clinical Accuracy of Four Continuous Glucose Monitors,” Diabetes Care 31:1160-1164 (2008); Russell et al., “Outpatient Glycemic Control with a Bionic Pancreas in Type 1 Diabetes,” N. Engl. J. Med. 371:313-325 (2014)). Alternatively, transplantation of insulin-producing cells represents a promising curative treatment for type 1 diabetes by providing patients with the cells that they have unfortunately lost (Shapiro et al., “Clinical Pancreatic Islet Transplantation,” Nature Reviews Endocrinology 13:268 (2017); Shapiro et al., “Islet Transplantation in Seven Patients with Type 1 Diabetes Mellitus Using a Glucocorticoid-free Immunosuppressive Regimen,” N. Engl. J. Med. 343:230-238 (2000); Posselt et al., “Islet Transplantation in Type 1 Diabetics using an Immunosuppressive Protocol Based on the Anti-LFA-1 Antibody Efalizumab,” Am. J. Transplant 10:1870-1880 (2010); Barton et al., “Improvement in Outcomes of Clinical Islet Transplantation: 1999-2010,” Diabetes Care 35:1436-1445 (2012); Ryan et al., “Five-year Follow-up After Clinical Islet Transplantation,” Diabetes 54:2060-2069 (2005)). Notably, more than 1,500 patients have been treated with human islet transplantation and achieved clinical success since 2000 (Shapiro et al., “Clinical Pancreatic Islet Transplantation,” Nature Reviews Endocrinology 13:268 (2017)). However, limited patients benefit from islet transplantation due to the requirement of chronic immune suppression and donor shortage.

Cell encapsulation (Scharp and Marchetti, “Encapsulated Islets for Diabetes Therapy: History, Current Progress, and Critical Issues Requiring Solution,” Adv. Drug Deliver. Rev. 67-68:35-73 (2014); Desai and Shea, “Advances in Islet Encapsulation Technologies,” Nat. Rev. Drug Discov. 16:338-350 (2017); Veiseh et al., “Managing Diabetes with Nanomedicine: Challenges and Opportunities,” Nat. Rev. Drug Discov. 14:45-57 (2015); Ernst et al., “Nanotechnology in Cell Replacement Therapies for Type 1 Diabetes,” Adv Drug Deliver Rev 139:116-138 (2019); Bowers et al., “Engineering the Vasculature for Islet Transplantation,” Acta Biomater. 95:131-151 (2019); Orive et al., “Cell Encapsulation: Promise and Progress,” Nat. Med. 9:104-107 (2003); Orive et al., “Cell Encapsulation: Technical and Clinical Advances,” Trends Pharmacol. Sci. 36:537-546 (2015)), designed to establish an immunological barrier against the host to protect transplanted cells while allowing free transfer of glucose, insulin, and essential nutrients, has been widely investigated for immunosuppression-free cell replacement therapies for T1D. This approach has become particularly attractive in recent years due to the progress that makes it possible to generate a limitless supply of insulin-producing β cells from stem cells (SC-β cells) (Veres et al., “Charting Cellular Identity During Human In Vitro β-cell Differentiation,” Nature 569:368-373 (2019); Sharon et al., “A Peninsular Structure Coordinates Asynchronous Differentiation with Morphogenesis to Generate Pancreatic Islets,” Cell 176:790-804.e713 (2019); Pagliuca et al., “Generation of Functional Human Pancreatic β Cells In Vitro,” Cell 159:428-439 (2014); Rezania et al., “Reversal of Diabetes with Insulin-producing Cells Derived In Vitro from Human Pluripotent Stem Cells,” Nat. Biotechnol. 32:1121 (2014); Guo et al., “Factors Expressed by Murine Embryonic Pancreatic Mesenchyme Enhance Generation of Insulin-producing Cells from hESCs,” Diabetes 62:1581-1592 (2013); Van Hoof et al., “Differentiation of Human Embryonic Stem Cells into Pancreatic Endoderm in Patterned Size-controlled Clusters,” Stem Cell Research 6:276-285 (2011); Hogrebe et al., “Targeting the Cytoskeleton to Direct Pancreatic Differentiation of Human Pluripotent Stem Cells,” Nat. Biotechnol. 38:460-470 (2020); Maxwell et al., “Gene-edited Human Stem cell-derived β Cells from a Patient with Monogenic Diabetes Reverse Preexisting Diabetes in Mice,” Science Translational Medicine 12 (2020); Nair et al., “Recapitulating Endocrine Cell Clustering in Culture Promotes Maturation of Human Stem-cell-derived β Cells,” Nat. Cell Biol. 21:263-274 (2019)), relieving the cadaveric donor tissue limitations and benefiting much broader patient populations. However, developing a clinically feasible, long-term functional cell encapsulation device is a significant, unmet challenge (Scharp and Marchetti, “Encapsulated Islets for Diabetes Therapy: History, Current Progress, and Critical Issues Requiring Solution,” Adv. Drug Deliver. Rev. 67-68:35-73 (2014); Desai and Shea, “Advances in Islet Encapsulation Technologies,” Nat. Rev. Drug Discov. 16:338-350 (2017); Veiseh et al., “Managing Diabetes with Nanomedicine: Challenges and Opportunities,” Nat. Rev. Drug Discov. 14:45-57 (2015); Ernst et al., “Nanotechnology in Cell Replacement Therapies for Type 1 Diabetes,” Adv Drug Deliver Rev 139:116-138 (2019)). Among the challenges, a major one is the foreign body response against the encapsulation device, which leads to cellular overgrowth and fibrotic deposition which consequently results in diminished mass transfer and graft failure (Scharp and Marchetti, “Encapsulated Islets for Diabetes Therapy: History, Current Progress, and Critical Issues Requiring Solution,” Adv. Drug Deliver. Rev. 67-68:35-73 (2014); Chang et al., “Nanoporous Immunoprotective Device for Stem-Cell-Derived β-Cell Replacement Therapy,” ACS Nano 11:7747-7757 (2017); Bose et al., “A Retrievable Implant for the Long-term Encapsulation and Survival of Therapeutic Xenogeneic Cells,” Nature Biomedical Engineering 4:814-826 (2020); Anderson et al., “Foreign Body Reaction to Biomaterials,” Semin. Immunol. 20:86-100 (2008); Harding and Reynolds, “Combating Medical Device Fouling,” Trends Biotechnol. 32:140-146 (2014); Grainger, D. W. J. N. b., “All Charged Up About Implanted Biomaterials,” Nat. Biotechnol. 31:507-509 (2013); Williams, D. F. J. B., “On the Mechanisms of Biocompatibility,” Biomaterials 29:2941-2953 (2008); Wick et al., “The Immunology of Fibrosis,” Annu. Rev. Immunol. 31:107-135 (2013); Wynn and Ramalingam, “Mechanisms of Fibrosis: Therapeutic Translation for Fibrotic Disease,” Nat. Med. 18:1028 (2012)). For example, the ViaCyte device (Kumagai-Braesch et al., “The TheraCyte™ Device Protects Against Islet Allograft Rejection in Immunized Hosts,” Cell Transplant 22:1137-1146 (2013); Haller et al., “Macroencapsulated Human iPSC-derived Pancreatic Progenitors Protect Against STZ-induced Hyperglycemia in Mice,” Stem Cell Reports 12:787-800 (2019)) and the Beta-Air device (Ludwig et al., “A Novel Device for Islet Transplantation Providing Immune Protection and Oxygen Supply,” Horm. Metab. Res. 42:918-922 (2010); Barkai et al., “Enhanced Oxygen Supply Improves Islet Viability in a New Bioartificial Pancreas,” Cell Transplant 22:1463-1476 (2013)), the two most advanced devices in the field, although promising in preventing allo- and auto-immune responses, failed to provide any clinical benefit or long-term cell function due to compromised mass transfer caused by foreign body response and fibrotic reactions (Bose et al., “A Retrievable Implant for the Long-term Encapsulation and Survival of Therapeutic Xenogeneic Cells,” Nature Biomedical Engineering 4:814-826 (2020); Pullen, L. C., “Stem Cell—Derived Pancreatic Progenitor Cells Have Now Been Transplanted into Patients: Report from IPITA 2018,” Am. J. Transplant 18:1581-1582 (2018); Liu et al., “Zwitterionically Modified Alginates Mitigate Cellular Overgrowth for Cell Encapsulation,” Nat. Commun. 10:1-14 (2019); Hentze et al., “Teratoma Formation by Human Embryonic Stem Cells: Evaluation of Essential Parameters for Future Safety Studies,” Stem Cell Research 2:198-210 (2009)).

Efforts have been made to tackle the challenge of foreign body responses, especially for the commonly used encapsulation material—alginate hydrogels (Dolgin, E., “Encapsulate This,” Nat. Med. 20:9-11 (2014); Vegas et al., “Combinatorial Hydrogel Library Enables Identification of Materials That Mitigate the Foreign Body Response in Primates,” Nat. Biotech. 34:345-352 (2016); Veiseh et al., “Size- and Shape-dependent Foreign Body Immune Response to Materials Implanted in Rodents and Non-human Primates,” Nat. Mater. 14:643-651 (2015); An et al., “Designing a Retrievable and Scalable Cell Encapsulation Device for Potential Treatment of Type 1 Diabetes,” Proc. Natl. Acad. Sci. USA 115(2):E263-E272 (2017); Lee and Mooney, “Alginate: Properties and Biomedical Applications,” Prog. Polym. Sci. 37:106-126 (2012); Kearney et al., “Macroscale Delivery Systems for Molecular and Cellular Payloads,” Nat. Mater. 12:1004-1017 (2013)). For example, combinational approaches have been employed to identify advanced alginate derivatives from 774 chemical modifications. Three “hits” substantially reduced cellular overgrowth on implanted alginate microcapsules in both mice and non-human primates (Vegas et al., “Combinatorial Hydrogel Library Enables Identification of Materials That Mitigate the Foreign Body Response in Primates,” Nat. Biotech. 34:345-352 (2016); Lee and Mooney, “Alginate: Properties and Biomedical Applications,” Prog. Polym. Sci. 37:106-126 (2012)). Our group developed fibrosis-mitigating alginate microcapsules using a different approach. In particular, alginates were modified with zwitterionic functional groups (Liu et al., “Zwitterionically Modified Alginates Mitigate Cellular Overgrowth for Cell Encapsulation,” Nat. Commun. 10:1-14 (2019)) known for their biofouling-resistant properties (Jiang et al., “Ultralow-fouling, Functionalizable, and Hydrolyzable Zwitterionic Materials and Their Derivatives for Biological Applications,” Adv. Mater. 22:920-932 (2010); Ladd et al., “Zwitterionic Polymers Exhibiting High Resistance to Nonspecific Protein Adsorption from Human Serum and Plasma,” Biomacromolecules 9:1357-1361 (2008); Zhang et al., “Zwitterionic Hydrogels Implanted in Mice Resist the Foreign-body Reaction,” Nat. Biotechnol. 31:553-556 (2013)) and reproducible and robust reduction of cellular overgrowth was observed in various models, including mice, dogs, and pigs. Microcapsules made of the zwitterionically modified alginates and used to encapsulate rat islets enabled long-term diabetes correction for up to 200 days in immunocompetent mice. Although these results are promising, the inability to reliably retrieve all transplanted microcapsules (Liu et al., “Zwitterionically Modified Alginates Mitigate Cellular Overgrowth for Cell Encapsulation,” Nat. Commun. 10:1-14 (2019); Bochenek et al., “Alginate Encapsulation as Long-term Immune Protection of Allogeneic Pancreatic Islet Cells Transplanted into the Omental Bursa of Macaques,” Nature Biomedical Engineering 2:810-821 (2018)), and the intrinsic weakness of hydrogel materials (Lee and Mooney, “Hydrogels for Tissue Engineering,” Chem. Rev. 101:1869-1879 (2001); Khademhosseini and Langer, “Microengineered Hydrogels for Tissue Engineering,” Biomaterials 28:5087-5092 (2007)) raise safety concerns for clinical applications. These concerns merit particular consideration when SC-β cells are used due to the potential risk of nontarget cells (Bose et al., “A Retrievable Implant for the Long-term Encapsulation and Survival of Therapeutic Xenogeneic Cells,” Nature Biomedical Engineering 4:814-826 (2020); An et al., “Designing a Retrievable and Scalable Cell Encapsulation Device for Potential Treatment of Type 1 Diabetes,” Proc. Natl. Acad. Sci. USA 115(2):E263-E272 (2017); Steele et al., “Therapeutic Cell Encapsulation Techniques and Applications in Diabetes,” Adv. Drug Deliver. Rev. 67:74-83 (2014); An et al., “Developing Robust, Hydrogel-based, Nanofiber-enabled Encapsulation Devices (NEEDs) for Cell Therapies,” Biomaterials 37:40-48 (2015)).

The present invention is directed to overcoming these and other deficiencies in the art.

### SUMMARY

A first aspect of the disclosure is directed to an implantable therapeutic delivery system comprising: a nanofiber core substrate having proximal and distal ends, said nanofiber core substrate having an interior nanofiber wall defining an internal space extending longitudinally along the core substrate, with one or more therapeutic agents positioned within the internal space; and a hydrogel surrounding said nanofiber core substrate, wherein said hydrogel comprises 0.1% to 20% of an alginate mixture, said alginate mixture comprising zwitterionically modified alginate and pure alginate in a ratio of 1:1000 to 1000:1 (v/v).

Another aspect of the disclosure is directed to an implantable therapeutic delivery system comprising: a nanofiber core substrate having proximal and distal ends, said nanofiber core substrate having an interior nanofiber wall defining an internal space extending longitudinally along the core substrate, with one or more therapeutic agents positioned within the internal space; and a biocompatible polymeric coating surrounding said nanofiber core substrate, wherein said biocompatible polymeric coating has a thickness of 1 nm to 5 mm, and wherein the standard deviation in thickness of the polymeric coating around the entirety of the nanofiber core substrate is <100%.

Another aspect of the present disclosure is directed to an implantable therapeutic delivery system comprising: a nanofiber core substrate having proximal and distal ends, said nanofiber core substrate defined by an inner nanofiber layer and an outer nanofiber layer surrounding the inner nanofiber layer, wherein the inner nanofiber layer has a nanofiber structure that differs from the nanofiber structure of the outer nanofiber layer, said nanofiber core substrate further comprising an internal space surrounded by the inner nanofiber layer of the substrate, with one or more therapeutic agents positioned within said internal space; and a biocompatible polymeric coating surrounding said nanofiber core substrate.

Another aspect of the present disclosure is directed to a method of delivering a therapeutic agent to a subject in need thereof. This method involves implanting any one of the implantable therapeutic delivery systems as described herein into the subject.

Another aspect of the present disclosure relates a method of producing an implantable therapeutic delivery system. This method involves providing a longitudinally extending nanofiber core substrate, said substrate having a proximal and a distal end, each proximal and distal end having an opening to at least one internal space within the nanofiber core substrate; sealing the proximal end of the nanofiber core substrate; soaking the sealed proximal end and outer surface of the nanofiber core substrate in a biocompatible polymer solution to allow polymer solution penetration into the nanofiber core substrate; filling the at least one internal space of the nanofiber core substrate with one or more crosslinking agents to crosslink the coated biocompatible polymer solution to the nanofiber core substrate; loading one or more therapeutic agents into the at least one internal space of the nanofiber core substrate through the opening at the distal end of the nanofiber core substrate; sealing the distal end of the loaded nanofiber core substrate; and coating the sealed distal end of the nanofiber core substrate with the biocompatible polymer solution to form the implantable therapeutic delivery system.

Another aspect of the present disclosure relates to a method of producing an implantable therapeutic delivery system. This method comprises: providing a longitudinally extending nanofiber core substrate, said substrate having a proximal and a distal end, each proximal and distal end having an opening to at least one internal space within the nanofiber core substrate; sealing the proximal end of the nanofiber core substrate; applying a biocompatible polymer solution to the sealed proximal end and outer surface of the nanofiber core substrate; loading one or more therapeutic agents into the at least one internal space of the nanofiber core substrate through the opening at the distal end of the nanofiber core substrate; sealing the distal end of the loaded nanofiber core substrate; applying the biocompatible polymer solution to the sealed distal end of the nanofiber core substrate; and crosslinking the coated biocompatible polymer solution to the nanofiber core substrate to form the implantable therapeutic delivery system.

Another aspect of the present disclosure relates to a method of producing an implantable therapeutic delivery system. This method involves providing a longitudinally extending nanofiber core substrate, said substrate having a proximal and a distal end, each proximal and distal end having an opening to at least one internal space within the nanofiber core substrate; sealing the proximal end of the nanofiber core substrate; loading one or more therapeutic agents into the at least one internal space of the nanofiber core substrate through the opening at the distal end of the nanofiber core substrate; sealing the distal end of the loaded nanofiber core substrate; soaking the sealed and loaded nanofiber core substrate in a cross-linker solution; coating the cross-linker soaked nanofiber core substrate with a biocompatible polymer solution; and crosslinking the coated biocompatible polymer solution to the nanofiber core substrate to form the implantable therapeutic delivery system.

Another aspect of the present disclosure is directed to a method of producing a porous nanofiber substrate. This method involves providing one or more polymer-solvent solutions; coating a rotating collecting rod with a viscous saccharide solution; electrospinning said one or more polymer solutions onto the coated rotating collecting rod to form the porous nanofiber substrate; and dissolving the viscous saccharide solution from the collecting rod, thereby removing the porous nanofiber substrate from the collecting rod.

A final aspect of the present disclosure is directed to a thermo sealing device. This thermos sealing device comprises a first substrate portion comprising a cut-out along its peripheral edge; a second substrate portion comprising a cut-out that is substantially identical in shape and size to the cut-out of the first substrate, said second substrate further comprising a trench configured to house a heating element, wherein said trench aligns with the cut-out of the second substrate; a connector connecting the first and second substrate portions in a manner that aligns the cut-out of the first substrate portion with the cut-out of the second substrate portion; and a heating element positioned in the trench of the second substrate portion

To mitigate the safety concerns while taking advantage of the superior biocompatibility of the zwitterionic alginates, herein is reported a Safe, Hypo-immunoreactive, Islet Encapsulation, Long-term-functional Device (termed as SHIELD) for delivery of islets and human SC-β cells. The SHIELD has several unique features. First, the design includes a concentric configuration where cells are encapsulated within the cylindrical wall allowing scale-up in both radial and longitudinal directions without sacrificing diffusion distance or mass transfer. Second, the strong and robust nanofibrous membrane with tunable, interconnected pore structure provides excellent mass transfer while ensuring safety. Third, an innovative “in-out” crosslinking strategy was developed to coat the nanofibrous membrane with a thin, uniform, controllable and stable layer of alginate hydrogel. Lastly, the zwitterionically modified alginate (Liu et al., “Zwitterionically Modified Alginates Mitigate Cellular Overgrowth for Cell Encapsulation,” Nat. Commun. 10:1-14 (2019), which is hereby incorporated by reference in its entirety) mitigates fibrotic reactions, enabling long-term function of SHIELD. Imaging, tensile, and peeling tests indicated that the “in-out” crosslinking resulted in an interpenetrating composite structure between the nanofibers and the alginate coating, exhibiting both high tensile strength and strong interfacial adhesion. In vitro and in vivo optimizations culminated in a device that prevented cell escape and cell penetration while supporting normal function of encapsulated cells. The facile mass transfer and the low level of fibrotic reaction enabled long-term restoration of normoglycemia (up to 399 days) in immunocompetent diabetic mice using encapsulated rat islets. More importantly, SHIELD encapsulating human SC-β cells corrected diabetes in SCID-Beige mice shortly after implantation for up to 238 days. Lastly, scalability and facile retrieval were achieved and demonstrated in dogs. This new device is translatable for cell therapies for T1D and other diseases.

### BRIEF DESCRIPTION OF THE DRAWINGS

FIGS. 1A-1O show the electrospinning of nanofiber tubes for SHIELD devices. FIG. 1A is a schematic diagram showing the electrospinning setup consisting of a rotating collector, a moving stage, and a high voltage power supply connected to an electrospinning nozzle. FIG. 1B is an image of nanofiber tubes with a length more than 20 cm. FIG. 1C is an image of nanofiber tubes with different diameters, scale bar, 5 mm. FIG. 1D is a plot of the nanofiber tube thickness as a function of electrospinning time. FIG. 1E is a plot of the pore size of nanofiber membrane (˜1.67 μm) weakly depending on thickness within the range investigated. FIGS. 1F-1J are SEM images of nanofiber membranes with different fiber diameters and pore sizes, scale bar, 2 μm (part of the data is also shown in FIG. 8). FIGS. 1K-1O are H&E images of uncoated devices after 14-day in vivo test in the intraperitoneal space of healthy C57BL6/J mice (n=4 or 5 for pore size, gray arrows point to the outer surface, while black arrows point to the inner surface; part of the data is also shown in FIG. 8), scale bar, 200 μm.

FIGS. 2A-2J show the “in-out crosslinking” method leading to robust alginate coating. FIGS. 2A-2D show the tensile test for dip-coated membranes and “in-out crosslinked” membranes. FIGS. 2A and 2B show delamination between the alginate hydrogel and nanofiber membrane that was observed for dip-coated membranes. FIGS. 2C and 2D show the “in-out crosslinked” membranes exhibited an excellent integration between alginate hydrogel and the nanofiber membrane during the tensile test, scale bars, 5 mm. FIG. 2E is the stress-strain curves for uncoated membranes, dip-coated membranes, and “in-out crosslinked” membranes. FIG. 2F is a SEM image showing the interpenetration between alginate and nanofibers for “in-out crosslinked” membranes, scale bar, 20 μm. FIGS. 2G-2J show the peeling test for “in-out crosslinked” membranes. FIGS. 2G-2I show the remaining nanofibers on the hydrogel after the peeling test verified the strong coating adhesion enabled by the “in-out crosslinking” method: FIG. 2G is aa cartoon depicting the peel test, FIGS. 2H and 2I are images from the peel test. FIG. 2J shows the force/width as a function of displacement for the peeling test, scale bars, 5 mm.

FIGS. 3A-3D show the thermo cutting device for SHIELD. FIGS. 3A and 3B are a schematic diagram and an image, respectively, showing the transparent thermo cutter consisting of a power supply (not shown), PDMS supporting materials, and a vertically oriented heating element made of iron-chrome-aluminum heating alloy (4 mm width), scale bar, 10 mm. FIGS. 3C and 3D show that using the custom-designed thermo cutter, a smooth arch shape sealing was achieved. This was used for the entire study to minimize sharp corners for SHIELD, scale bars, 5 mm.

FIGS. 4A-4R show the scalability and retrievability of SHIELD in dogs. FIG. 4A is an image of a hanging-suture SHIELD device before implantation (4%, 3:7 modified alginate coating, length ˜12 cm), scale bar, 5 mm. FIG. 4B is an image showing one end of a hanging-suture SHIELD device was bonded to a nylon suture with the arrow pointing to translucent thermo bonded area, scale bar, 5 mm. FIGS. 4C-4F are images showing the anchoring process through a suture grasper; white arrows point to the suture grasper, black arrows point to the hanging suture connecting the SHIELD, and gray arrows point to the SHIELD being delivered through a trocar, scale bars, 5 mm: (FIG. 4C) open the grasper; (FIG. 4D) catch the hanging suture; (FIG. 4E) withdraw the grasper together with the hanging suture; (FIG. 4F) the device anchored to the peritoneal layer of the body wall by the hanging suture. FIGS. 4G-4I are images showing a device after 1-month implantation: (FIG. 4G) adhesion to omentum occurred on one end; (FIGS. 4H and 4I) the majority of the device was free of adhesion as shown by black arrows. FIGS. 4J-4R are images of the retrieved device after 1-month implantation: (FIG. 4J) an image showing the mild adhesion to one end of the device, scale bar, 10 mm; (FIGS. 4K-4O) H&E images of the entire device, scale bar, 1 mm. FIGS. 4P and 4Q are images showing minimal cellular overgrowth on the surface of coated alginate hydrogel; gray arrows point to the outer surface, while black arrows point to the nanofiber membrane (the black area in FIG. 4P is also nanofiber membrane): (FIG. 4P) an optical image, scale bar, 1 mm; (FIG. 4Q) a H&E image, scale bar, 200 μm. FIG. 4R is an image showing the cellular overgrowth in the area having omentum adhesion; gray arrows point to the cellular overgrowth, while black arrows point to the nanofiber membrane, scale bar, 200 μm.

FIGS. 5A-5J show the design and fabrication of the SHIELD device. FIG. 1A is a schematic diagram showing the SHIELD device consisting of an inner nanofibrous tube and an outer nanofibrous tube. The inner nanofibrous tube keeps the clusters of insulin-producing cells around the inner surface of the outer nanofibrous tube and thus maintains a short diffusion distance. The outer nanofibrous tube is coated with a zwitterionic alginate hydrogel for fibrosis mitigation. FIGS. 5B-5E show the fabrication of the SHIELD device: (FIG. 5B) a schematic diagram showing the process of loading islets/alginate mixture onto the outer surface of the inner nanofibrous tube; (FIG. 5C) a representative image of the inner nanofibrous tube loaded with islets (the black area is nanofiber membrane), scale bar, 200 μm; (FIG. 5D) a schematic diagram of the in-out crosslinking method for fabricating the outer nanofibrous tube, which can generate a uniform and stable coating with a controllable thickness; (FIG. 5E) the SHIELD device was achieved after inserting the inner nanofibrous tube (loaded with islets) to the coated outer nanofibrous tube followed by thermo sealing. FIG. 5F is a SEM image showing the interconnected porous structure of nanofiber membranes, scale bar, 20 μm. FIG. 5G shows the chemical structure of zwitterionic alginate. FIG. 5H is an optical image showing the uniformity of the coated alginate hydrogel fabricated by the in-out crosslinking method (the black area is nanofiber membrane; the transparent area is alginate hydrogel), scale bar, 200 μm. FIG. 5I is a representative image of a rodent-size SHIELD (length ˜2.5 cm), scale bar, 5 mm. FIG. 5J is a representative image of a long SHIELD (length ˜10 cm), scale bar, 5 mm.

FIGS. 6A-6F show the control of coating thickness through diffusion time. FIG. 6A is a representative image of the SHIELD device showing the uniformity of alginate coating, scale bar, 2 mm. FIGS. 6B-6E show the coating thickness was controlled by adjusting the diffusion time: (FIG. 6B) 30 s; (FIG. 6C) 90 s; (FIG. 6D) 150 s; (FIG. 6E) 210 s, scale bar, 200 μm. FIG. 6F is a plot of the coating thickness as a function of diffusion time.

FIGS. 7A-7F show the quantification of the mechanical properties of uncoated membranes, dip-coated membranes, and in-out crosslinked membranes. FIGS. 7A-7B are graphical comparisons between uncoated membranes and dip-coated membranes (at the second breaking point): (FIG. 7A) tensile strength; (FIG. 7B) tensile strain. FIGS. 7C-7F are graphical comparisons between dip-coated membranes and in-out crosslinked membranes: (FIG. 7C) Young's modulus; (FIG. 7D) tensile strength; (FIGS. 7E and 7F) tensile strain.

FIGS. 8A-8J show the optimization of the pore size by balancing safety and mass transfer. FIG. 8A is a plot of pore size as a function of fiber diameter, scale bars, 2 μm. FIG. 8B is a graph of the number of samples having cell escape for uncoated devices with different pore sizes (average pore size: 0.15 μm, 0.38 μm, 0.67 μm, 1.05 μm, and 1.67 μm). FIG. 8C is a plot of the fluorescence units as a function of days post incubation for the presto blue test. FIG. 8D is a Live/Dead image of NIH3T3 cells inside a coated device after 2-day incubation, scale bar, 200 μm. FIGS. 8E-8G are H&E images of uncoated devices after 14-day in vivo test in the intraperitoneal space of healthy C57BL6/J mice (n=4 or 5 for each pore size, gray arrows point to the outer surface of devices, while black arrows point to the inner surface), scale bars, 200 μm: (FIG. 8E) 1.67 μm; (FIG. 8F) 0.67 μm; (FIG. 8G) 0.15 μm. FIGS. 8H-8J are graphs showing the quantification of (FIG. 8H) cell penetration, (FIG. 8I) thickness of fibrotic layer, and (FIG. 8J) number of samples having tissue adhesion for uncoated devices with different pore sizes.

FIGS. 9A-9Y show the in vitro cell escape test of uncoated devices. FIGS. 9A-9E are images from Day 2. FIGS. 9F-9J are images from Day 5. FIGS. 9K-9O are images from Day 10. FIGS. 9P-9T are images from Day 14, scale bar, 1 mm. FIGS. 9U-9Y are images of NIH3T3/GFP cells in the devices after 14-day incubation, scale bar, 200 μm.

FIGS. 10A-10Y show the in vitro cell escape test of coated devices. FIGS. 10A-10E are images from Day 2. FIGS. 10F-10J are images from Day 5. FIGS. 10K-10O are images from Day 10. FIGS. 10P-10T are images from Day 14, scale bar, 1 mm. FIGS. 10U-10Y are images of NIH3T3/GFP cells in the devices after 14-day incubation, scale bar, 200 μm.

FIGS. 11A-11V show the results from the in vitro cell escape test of uncoated and coated devices with a pore size of 1.67 μm. FIGS. 11A-11K show that the cell escape started to occur on day 5 post incubation for uncoated devices. FIGS. 11L-11V show that cell escape was not detected from coated SHIELD devices. Scale bars, white 1 mm, black 200 μm.

FIGS. 12A-12L show stable zwitterionic alginate coating resulting in superior biocompatibility. FIGS. 12A-12B are images of in vitro cell attachment test on alginate hydrogel coating surface using NIH3T3/GFP cells, scale bars, 1 mm. FIGS. 12C-12F are representative images of devices coated with alginate hydrogels after 14-day in vivo test in the intraperitoneal space of healthy C57BL63 mice; gray arrows point to the outer surface of coated devices, while black arrows point to the nanofiber membranes (the black areas in FIG. 12C and FIG. 12D are also nanofiber membranes), scale bars, 200 μm. FIGS. 12A, 12C, and 12E used 3% SLG100. FIGS. 12B, 12D, and 12F used 3% modified alginate (SB-alginate:SLG100=3:7). FIG. 12G is a graph of the quantification of the cell attachment on the device after 1 day incubation. FIGS. 12H and 12I are graphs of the quantification of coating stability for alginate hydrogels with different ratios between SB-alginate and unmodified high molecular weight alginate SLG100 (n=4 for each ratio, combinations of devices retrieved on day 14 and day 28, 0:10 represents neat SLG100): (FIG. 12H) 4% alginate; (FIG. 12I) 3% alginate. FIGS. 12J and 12K are graphs of the number of samples having (FIG. 12J) cell penetration and (FIG. 12K) tissue adhesion for uncoated devices, devices coated with neat SLG100 and modified alginate. Neat SLG100 (n=8) is a combination of samples from concentration of 3% and 4%, while modified alginate (n=20) is a combination of samples from 3% modified alginate (3:7 & 5:5) and 4% modified alginate (3:7, 5:5 & 7:3). FIG. 12L is a graph of the quantification of cellular overgrowth on coated devices. Neat SLG100 (n=8) is a combination of samples from concentration of 3% and 4%, while modified alginate (n=12) is a combination of samples from 3% (3:7) and 4% (3:7 & 5:5).

FIGS. 13A-13L are representative images of SHIELD devices coated with 4% alginate hydrogels after 14-day in vivo test in the intraperitoneal space of healthy C57BL6/J mice. SB-alginate/SLG100 ratio: (FIGS. 13A-13C) 7:3; (FIGS. 13D-13F) 5:5; (FIG. 13G-13I) 3:7; (FIG. 13J-13L) 0:10 (n=4 for each ratio, retrieved on day 14 and day 28, 0:10 represents neat SLG100. Gray arrows point to the outer surface. While black arrows point to the nanofiber membrane (the black area is also nanofiber membrane), white arrows point to fibrosis due to alginate detachment). Scale bars, white 1 mm, black 200 μm.

FIGS. 14A-14I show representative images of SHIELD devices coated with 3% alginate hydrogels after 14-day in vivo test in the intraperitoneal space of healthy C57BL6/J mice. SB-alginate/SLG100 ratio: (FIGS. 14A-14C) 5:5; (FIGS. 14D-14F) 3:7; (FIGS. 14G-14I) 0:10 (0:10 represents neat SLG100), n=4 for each ratio, retrieved on day 14 and day 28. Gray arrows point to the outer surface. While black arrows point to the nanofiber membrane (the black area is also nanofiber membrane), white arrows point to fibrosis due to alginate detachment, part of the data is also shown in FIG. 12). Scale bars, white 1 mm, black 200 μm.

FIGS. 15A-15G show SHIELD supports long-term function of rat islets in C57BL6/J mice. FIG. 15A is a plot of the blood glucose as a function of days post implantation (the retrieval is indicated by arrows together with dash lines corresponding to blood glucose curves). FIG. 15B is a plot of the OGTT for healthy mice (n=5), diabetic mice treated with modified alginate coated devices (a combination of all modified alginate coated devices longer than 193 days, n=8) and uncoated devices (on day 50, n=3). FIGS. 15C and 15D are images of a SHIELD device retrieved on day 325 showing rare cellular overgrowth, gray arrows point to the outer surface, while black arrows point to the nanofiber membrane (the black area in FIG. 15C is also nanofiber membrane), scale bars, 200 μm. FIG. 15C is an optical image; FIG. 15D is an H&E image. FIGS. 15E-15G are images of islets in the SHIELD device retrieved on day 325: (FIG. 15E) an optical image, scale bar, 10 mm; (FIG. 15F) an H&E image, scale bar, 100 μm; (FIG. 15G) insulin/glucagon/DAPI staining, scale bar, 25 μm.

FIGS. 16A-16I show the data from in vivo test of SHIELD devices in C57BL6/J mice using rat islets. FIG. 16A is a plot of the body weight as a function of days post implantation. FIG. 16B is a graph of the body weight increase of mice treated with uncoated (n=3) and modified alginate coated (n=14) devices ˜50 days post implantation. FIG. 16C is a plot of the number of samples having tissue adhesion for uncoated devices (n=3) and coated ones (a combination of 3% and 4% modified alginate 3:7, n=15). FIG. 16D is a plot of body weight before and after retrieval (a combination of 3% and 4% modified alginate coated devices that maintained normoglycemia until retrieval, n=7). FIG. 16E is a graph off the insulin secretion of retrieved devices from the ex vivo GSIS test (a combination of modified alginate coated devices that maintained normoglycemia until retrieval, n=7). FIGS. 16F-16G show the quantification of coating stability for SHIELD devices with rat islets (3% (82-274 days, n=4) and 4% (34-399 days, n=11) modified alginate coating). FIG. 16H is a graph of the quantification of cellular overgrowth for SHIELD devices with islets (34-399 days, n=15) and without islets (a combination of 3% and 4% modified alginate 3:7, 14-28 days, n=8). FIG. 16I is a graph of the quantification of cellular overgrowth on functional (n=9) and failed (n=6) devices (a combination of devices coated with 3% and 4% modified alginate 3:7.

FIGS. 17A-17D show the characterization of human SC-β cells. FIG. 17A shows uniform clusters (˜150 μm) of human SC-β cells prepared by the aggregation process before encapsulation, scale bar, 400 μm. FIGS. 17B-17D are clusters of human SC-β cells from a retrieved device from the mouse that accidentally died after blood collection on day 234, scale bars, 100 μm; (FIG. 17B) an H&E image; (FIG. 17C) insulin/glucagon/DAPI staining (FIG. 17D) C-peptide/PDX1/DAPI staining.

FIGS. 18A-18G show SHIELD supports long-term function of human SC-β cells in SCID-beige mice. FIG. 18A is a plot of blood glucose as a function of days post implantation (The retrieval is indicated by gray arrows together with dash lines corresponding to blood glucose curves). FIG. 18B is a plot of the OGTT for diabetic mice (n=4) and mice having engrafted devices (day 45 and day 61, n=9). FIGS. 18C and 18D are images of the SHIELD device retrieved on day 222 showing mild cellular overgrowth, gray arrows point to the outer surface, while black arrows point to the nanofiber membrane (the black area in FIG. 18C is also nanofiber membrane), scale bars, 200 μm. FIG. 18C is an optical image; FIG. 18D is an H&E image. FIGS. 18E-18G are images of islets in the SHIELD device retrieved on day 238, scale bars, 100 μm: (FIG. 18E) an H&E image; (FIG. 18F) insulin/glucagon/DAPI staining; (FIG. 18G) C-peptide/PDX1/DAPI staining.

FIGS. 19A-19F show the data from in vivo test of SHIELD devices in SCID-beige mice using human SC-β cells. FIG. 19A is a plot of body weight as a function of days post implantation. FIG. 19B is a graph showing the body weight increase in ˜50 days post implantation for diabetic mice receiving no treatment (n=4) or treated with SHIELD devices (n=14). FIG. 19C is a graph of human C-peptide measured from mouse serum for short-term (day 45 and day 61, n=9) and long-term (day 172 and day 234, n=5). FIG. 20D is a plot of body weight before and after retrieval (coated devices that maintained normoglycemia until retrieval, n=10). FIGS. 19E-19F show the quantification of (FIG. 19E) coating stability and (FIG. 19F) cellular overgrowth for SHIELD devices with SC-β cells (≥36 days, n=15) or without cells (from 3% 3:7 coating stability test, 14-28 days, n=4).

FIGS. 20A-20D show SHIELD devices used in the intraperitoneal space of dogs. FIGS. 20A and 20B are images showing a hanging-suture SHIELD device having mild adhesion to omentum (half device) after 1-month of implantation. FIGS. 20C and 20D are images showing a non-anchored SHIELD device having adhesion to omentum at both ends after 1-month implantation, scale bar, 10 mm.

### DETAILED DESCRIPTION

The present disclosure relates to an implantable nanofiber-enabled therapeutic delivery system, methods of producing the delivery systems, and methods of using the same.

A first aspect of the disclosure is directed to an implantable therapeutic delivery system comprising: a nanofiber core substrate having proximal and distal ends, said nanofiber core substrate having an interior nanofiber wall defining an internal space extending longitudinally along the core substrate, with one or more therapeutic agents positioned within the internal space; and a hydrogel surrounding said nanofiber core substrate, wherein said hydrogel comprises 0.1% to 20% of an alginate mixture, said alginate mixture comprising zwitterionically modified alginate and pure alginate in a ratio of 1:1000 to 1000:1 (v/v).

The hydrogel surrounding the nanofiber core substrate may have a concentration of the alginate mixture ranging from about 0.1%, 1%, 2%, 3%, 4%, 5%, 6%, 7%, 8%, 9%, 10%, 11%, 12%, 13%, 14%, 15%, 16%, 17%, 18%, or 19%, up to about 1%, 2%, 3%, 4%, 5%, 6%, 7%, 8%, 9%, 10%, 11%, 12%, 13%, 14%, 15%, 16%, 17%, 18%, 19%, or 20%. In any embodiment, the hydrogel comprises 0.5% to about 10% of an alginate mixture. In any embodiment, the hydrogel comprises 0.1% to about 7% of an alginate mixture. A particularly useful hydrogel comprises 1% to 4% of an alginate mixture.

In accordance with this aspect of the disclosure, the alginate mixture of the hydrogel surrounding the nanofiber core substrate of the delivery system may comprise a ratio of zwitterionically modified alginate and pure alginate in a range from about 1; 10; 20; 30; 40; 50; 60; 70; 80; 90; 100; 200; 300; 400; 500; 600; 700; 800; 900; or 1,000 to about 1; 10; 20; 30; 40; 50; 60; 70; 80; 90; 100; 200; 300; 400; 500; 600; 700; 800; 900; or 1,000. In one embodiment, the alginate mixture comprises zwitterionically modified alginate and pure alginate in a ratio of 7:3 to 3:7 (v/v). The ratio of zwitterionically modified alginate to pure alginate may, for example, be 7:3, 6:4, 5:5 (1:1), 4:6, or 3:7 (v/v).

In accordance with this aspect of the disclosure, suitable zwitterionically modified alginates include, without limitation, those disclosed in Liu et al., “Zwitterionically Modified Alginates Mitigate Cellular Overgrowth for Cell Encapsulation,” Nat. Commun. 10(1):5262 (2019); and U.S. Patent Application Publication No. 20190389979 to Ma and Liu, which are hereby incorporated by reference in their entirety.

In any embodiment, the hydrogel surrounding the nanofiber core substrate of the implantable therapeutic delivery system as described herein, is crosslinked and interlocked to the nanofiber core substrate. In any embodiment, the hydrogel surrounding the nanofiber core substrate has a thickness of 1 nm to 5 mm, wherein the standard deviation in thickness of the hydrogel around the entirety of the nanofiber core substrate is <100%. In any embodiment, the standard deviation in thickness of the polymeric coating around the entirety of the nanofiber core substrate is <60%. The hydrogel may, for example, have a thickness ranging from about 1 nm; 10 nm; 100 nm; 200 nm; 300 nm; 400 nm; 500 nm; 600 nm; 700 nm; 800 nm; 900 nm; 1.00 nm; 2,000 nm; 3,000 nm; 4,000 nm; 5,000 nm; 6,000 nm; 7,000 nm; 8,000 nm; 9,000 nm; 10,000 nm; 20,000 nm; 30,000 nm; 40,000 nm; 50,000 nm; 60,000 nm; 70,000 nm; 80,000 nm; 90,000 nm; 100,000 nm; 200,000 nm; 300,000 nm; 400,000 nm; 500,000 nm; 600,000 nm; 700,000 nm; 800,000 nm; 900,000 nm; 1 mm; 2 mm; 3 mm; or 4 mm up to about 10 nm; 100 nm; 200 nm; 300 nm; 400 nm; 500 nm; 600 nm; 700 nm; 800 nm; 900 nm; 1.00 nm; 2,000 nm; 3,000 nm; 4,000 nm; 5,000 nm; 6,000 nm; 7,000 nm; 8,000 nm; 9,000 nm; 10,000 nm; 20,000 nm; 30,000 nm; 40,000 nm; 50,000 nm; 60,000 nm; 70,000 nm; 80,000 nm; 90,000 nm; 100,000 nm; 200,000 nm; 300,000 nm; 400,000 nm; 500,000 nm; 600,000 nm; 700,000 nm; 800,000 nm; 900,000 nm; 1 mm; 2 mm; 3 mm; 4 mm; or 5 mm.

In any embodiment, the hydrogel surrounding the nanofiber core substrate comprises one or more biologically active agents selected from the group consisting of a protein, peptide, antibody or antibody fragment thereof, antibody mimetic, a nucleic acid, a small molecule, a hormone, a growth factor, an angiogenic factor, a cytokine, an anti-inflammatory agent, and combinations thereof. Suitable anti-inflammatory agents include, without limitation, non-steroidal anti-inflammatory drugs (NSAID) (e.g., diclofenac, diflunisal, etodolac, fenoprofen, flurbiprofen, ibuprofen, indomethacin, ketoprofen, ketorolac, mefenamic acid, meloxicam, nabumetone, naproxen, oxaprozin, piroxicam, salsalate, sulindac, and tolmetin), analgesics (e.g., acetaminophen, oxycodone, tramadol, and propoxyphene hydrochloride), glucocorticoids (e.g., cortisone, dexamethasone, hydrocortisone, methylprednisolone, prednisolone, and prednisone), and dihydrofolate reductase inhibitors (e.g., methotrexate).

Another aspect of the disclosure is directed to an implantable therapeutic delivery system comprising: a nanofiber core substrate having proximal and distal ends, said nanofiber core substrate having an interior nanofiber wall defining an internal space extending longitudinally along the core substrate, with one or more therapeutic agents positioned within the internal space; and a biocompatible polymeric coating surrounding said nanofiber core substrate, wherein said biocompatible polymeric coating has a thickness of 1 nm to 5 mm, and wherein the standard deviation in thickness of the polymeric coating around the entirety of the nanofiber core substrate is <100%. In one embodiment, the standard deviation in thickness of the polymeric coating around the entirety of the nanofiber core substrate is <60%.

In any embodiment, the interior nanofiber wall of the nanofiber core substrate of this implantable therapeutic delivery system as described herein forms a tube having a diameter of 0.1 mm to 30 cm. The diameter of the tube may, for example, range from about 0.1 mm, 1 mm, 10 mm, 20 mm, 30 mm, 40 mm, 50 mm, 60 mm, 70 mm, 80 mm, 90 mm, 100 mm, 110 mm, 120 mm, 130 mm, 140 mm, 150 mm, 160 mm, 170 mm, 180 mm, 190 mm, 200 mm, 210 mm, 220 mm, 230 mm, 240 mm, 250 mm, 260 mm, 270 mm, 280 mm, or 290 mm, up to about 1 mm, 10 mm, 20 mm, 30 mm, 40 mm, 50 mm, 60 mm, 70 mm, 80 mm, 90 mm, 100 mm, 110 mm, 120 mm, 130 mm, 140 mm, 150 mm, 160 mm, 170 mm, 180 mm, 190 mm, 200 mm, 210 mm, 220 mm, 230 mm, 240 mm, 250 mm, 260 mm, 270 mm, 280 mm, 290 mm, or 300 mm. In any embodiment, the tube is a conical tube. In any embodiment, the tube is a cylindrical tube.

In any embodiment, the interior wall of the implantable therapeutic delivery system as described herein has a thickness of 1 μm to 5 mm. The interior wall may, for example, have a thickness ranging from about 1 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1,000 μm, 2 mm, 3 mm, or 4 mm, up to about 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1,000 μm, 2 mm, 3 mm, 4 mm or 5 mm.

In any embodiment, the nanofiber core substrate of the implantable therapeutic delivery system as described herein has a nanofiber density of 0.01 g/cm³ to 1.5 g/cm³. The nanofiber density may, for example, range from about 0.01 g/cm³, 0.05 g/cm³, 0.1 g/cm³, 0.15 g/cm³, 0.20 g/cm³, 0.25 g/cm³, 0.30 g/cm³, 0.35 g/cm³, 0.40 g/cm³, 0.45 g/cm³, 0.50 g/cm³, 0.55 g/cm³, 0.60 g/cm³, 0.65 g/cm³, 0.70 g/cm³, 0.75 g/cm³, 0.80 g/cm³, 0.85 g/cm³, 0.90 g/cm³, 0.95 g/cm³, 1.00 g/cm³, 1.05 g/cm³, 1.10 g/cm³, 1.15 g/cm³, 1.20 g/cm³, 1.25 g/cm³, 1.30 g/cm³, 1.35 g/cm³, 1.40 g/cm³, or 1.45 g/cm³ up to about 0.05 g/cm³, 0.1 g/cm³, 0.15 g/cm³, 0.20 g/cm³, 0.25 g/cm³, 0.30 g/cm³, 0.35 g/cm³, 0.40 g/cm³, 0.45 g/cm³, 0.50 g/cm³, 0.55 g/cm³, 0.60 g/cm³, 0.65 g/cm³, 0.70 g/cm³, 0.75 g/cm³, 0.80 g/cm³, 0.85 g/cm³, 0.90 g/cm³, 0.95 g/cm³, 1.00 g/cm³, 1.05 g/cm³, 1.10 g/cm³, 1.15 g/cm³, 1.20 g/cm³, 1.25 g/cm³, 1.30 g/cm³, 1.35 g/cm³, 1.40 g/cm³, 1.45 g/cm³, or 1.50 g/cm³.

In any embodiment, nanofibers of the nanofiber core substrate of the implantable therapeutic delivery system as described herein have a diameter of 1 nm to 50 μm. The nanofiber diameter may, for example, range from about 1 nm; 10 nm; 20 nm; 30 nm; 40 nm; 50 nm; 60 nm; 70 nm; 80 nm; 90 nm; 100 nm; 200 nm; 300 nm; 400 nm; 500 nm; 600 nm; 700 nm; 800 nm; 900 nm; 1,000 nm; 2,000 nm; 3,000 nm; 4,000 nm; 5,000 nm; 6,000 nm; 7,000 nm; 8,000 nm; 9,000 nm; 10,000 nm; 20,000 nm; 30,000 nm; or 40,000 nm up to about 10 nm; 20 nm; 30 nm; 40 nm; 50 nm; 60 nm; 70 nm; 80 nm; 90 nm; 100 nm; 200 nm; 300 nm; 400 nm; 500 nm; 600 nm; 700 nm; 800 nm; 900 nm; 1,000 nm; 2,000 nm; 3,000 nm; 4,000 nm; 5,000 nm; 6,000 nm; 7,000 nm; 8,000 nm; 9,000 nm; 10,000 nm; 20,000 nm; 30,000 nm; 40,000 nm; or 50,000 nm.

In any embodiment, the nanofiber core substrate comprises pores, said pores having a diameter of 1 nm to 50 μm. The pore diameter may, for example, range from about 1 nm; 10 nm; 20 nm; 30 nm; 40 nm; 50 nm; 60 nm; 70 nm; 80 nm; 90 nm; 100 nm; 200 nm; 300 nm; 400 nm; 500 nm; 600 nm; 700 nm; 800 nm; 900 nm; 1,000 nm; 2,000 nm; 3,000 nm; 4,000 nm; 5,000 nm; 6,000 nm; 7,000 nm; 8,000 nm; 9,000 nm; 10,000 nm; 20,000 nm; 30,000 nm; or 40,000 nm up to 10 nm; 20 nm; 30 nm; 40 nm; 50 nm; 60 nm; 70 nm; 80 nm; 90 nm; 100 nm; 200 nm; 300 nm; 400 nm; 500 nm; 600 nm; 700 nm; 800 nm; 900 nm; 1,000 nm; 2,000 nm; 3,000 nm; 4,000 nm; 5,000 nm; 6,000 nm; 7,000 nm; 8,000 nm; 9,000 nm; 10,000 nm; 20,000 nm; 30,000 nm; 40,000 nm or 50,000 nm.

In any embodiment, the nanofiber composition of the nanofiber core substrate of the implantable therapeutic delivery system as described herein is homogeneous. In any embodiment, the nanofiber composition of the nanofiber core substrate is heterogeneous.

Another aspect of the present disclosure is directed to an implantable therapeutic delivery system comprising: a nanofiber core substrate having proximal and distal ends, said nanofiber core substrate defined by an inner nanofiber layer and an outer nanofiber layer surrounding the inner nanofiber layer, wherein the inner nanofiber layer has a nanofiber structure that differs from the nanofiber structure of the outer nanofiber layer, said nanofiber core substrate further comprising an internal space surrounded by the inner nanofiber layer of the substrate, with one or more therapeutic agents positioned within said internal space; and a biocompatible polymeric coating surrounding said nanofiber core substrate.

In accordance with this aspect of the disclosure, the nanofiber core substrate optionally comprises one or more middle nanofiber layers positioned between the inner and outer nanofiber layers of the substrate, each middle nanofiber layer comprising a nanofiber structure that differs from the nanofiber structure of the inner and outer nanofiber layers.

In any embodiment, the nanofiber substrate of this implantable therapeutic delivery system as described herein is a cylindrical tube. In some embodiment, the cylindrical tube has a diameter of 0.1 mm to 30 cm. The diameter of the cylindrical tube may, for example, range from about 0.1 mm, 1 mm, 10 mm, 20 mm, 30 mm, 40 mm, 50 mm, 60 mm, 70 mm, 80 mm, 90 mm, 100 mm, 110 mm, 120 mm, 130 mm, 140 mm, 150 mm, 160 mm, 170 mm, 180 mm, 190 mm, 200 mm, 210 mm, 220 mm, 230 mm, 240 mm, 250 mm, 260 mm, 270 mm, 280 mm, or 290 mm, to 1 mm, 10 mm, 20 mm, 30 mm, 40 mm, 50 mm, 60 mm, 70 mm, 80 mm, 90 mm, 100 mm, 110 mm, 120 mm, 130 mm, 140 mm, 150 mm, 160 mm, 170 mm, 180 mm, 190 mm, 200 mm, 210 mm, 220 mm, 230 mm, 240 mm, 250 mm, 260 mm, 270 mm, 280 mm, or 290 mm up to about 1 mm, 10 mm, 20 mm, 30 mm, 40 mm, 50 mm, 60 mm, 70 mm, 80 mm, 90 mm, 100 mm, 110 mm, 120 mm, 130 mm, 140 mm, 150 mm, 160 mm, 170 mm, 180 mm, 190 mm, 200 mm, 210 mm, 220 mm, 230 mm, 240 mm, 250 mm, 260 mm, 270 mm, 280 mm, or 290 mm, to 1 mm, 10 mm, 20 mm, 30 mm, 40 mm, 50 mm, 60 mm, 70 mm, 80 mm, 90 mm, 100 mm, 110 mm, 120 mm, 130 mm, 140 mm, 150 mm, 160 mm, 170 mm, 180 mm, 190 mm, 200 mm, 210 mm, 220 mm, 230 mm, 240 mm, 250 mm, 260 mm, 270 mm, 280 mm, 290 mm or 300 mm. In any embodiment, the nanofiber substrate is a conical tube.

In any embodiment, the nanofibers of the inner nanofiber layer and outer nanofiber layer of the implantable therapeutic delivery system as described herein independently have a diameter of 1 nm to 50 μm. The nanofiber diameter may, for example, be about 1 nm; 10 nm; 20 nm; 30 nm; 40 nm; 50 nm; 60 nm; 70 nm; 80 nm; 90 nm; 100 nm; 200 nm; 300 nm; 400 nm; 500 nm; 600 nm; 700 nm; 800 nm; 900 nm; 1,000 nm; 2,000 nm; 3,000 nm; 4,000 nm; 5,000 nm; 6,000 nm; 7,000 nm; 8,000 nm; 9,000 nm; 10,000 nm; 20,000 nm; 30,000 nm; or 40,000 nm up to about 10 nm; 20 nm; 30 nm; 40 nm; 50 nm; 60 nm; 70 nm; 80 nm; 90 nm; 100 nm; 200 nm; 300 nm; 400 nm; 500 nm; 600 nm; 700 nm; 800 nm; 900 nm; 1,000 nm; 2,000 nm; 3,000 nm; 4,000 nm; 5,000 nm; 6,000 nm; 7,000 nm; 8,000 nm; 9,000 nm; 10,000 nm; 20,000 nm; 30,000 nm; 40,000 nm or 50,000 nm.

In any embodiment, the inner nanofiber layer and the outer nanofiber layer of the implantable therapeutic delivery system as described herein independently have a nanofiber density of 0.01 g/cm³ to 1.5 g/cm³. The nanofiber density of the inner nanofiber layer and/or the outer nanofiber layer may, for example, range from about 0.01 g/cm³, 0.05 g/cm³, 0.1 g/cm³, 0.15 g/cm³, 0.20 g/cm³, 0.25 g/cm³, 0.30 g/cm³, 0.35 g/cm³, 0.40 g/cm³, 0.45 g/cm³, 0.50 g/cm³, 0.55 g/cm³, 0.60 g/cm³, 0.65 g/cm³, 0.70 g/cm³, 0.75 g/cm³, 0.80 g/cm³, 0.85 g/cm³, 0.90 g/cm³, 0.95 g/cm³, 1.00 g/cm³, 1.05 g/cm³, 1.10 g/cm³, 1.15 g/cm³, 1.20 g/cm³, 1.25 g/cm³, 1.30 g/cm³, 1.35 g/cm³, 1.40 g/cm³, or 1.45 g/cm³ up to about 0.05 g/cm³, 0.1 g/cm³, 0.15 g/cm³, 0.20 g/cm³, 0.25 g/cm³, 0.30 g/cm³, 0.35 g/cm³, 0.40 g/cm³, 0.45 g/cm³, 0.50 g/cm³, 0.55 g/cm³, 0.60 g/cm³, 0.65 g/cm³, 0.70 g/cm³, 0.75 g/cm³, 0.80 g/cm³, 0.85 g/cm³, 0.90 g/cm³, 0.95 g/cm³, 1.00 g/cm³, 1.05 g/cm³, 1.10 g/cm³, 1.15 g/cm³, 1.20 g/cm³, 1.25 g/cm³, 1.30 g/cm³, 1.35 g/cm³, 1.40 g/cm³, 1.45 g/cm³, or 1.50 g/cm³.

In any embodiment, the inner nanofiber layer and the outer nanofiber layer of the implantable therapeutic delivery system as described herein independently have an average thickness of 1 μm to 5 mm. The inner nanofiber layer and the outer nanofiber layer may, for example, have a thickness of about 1 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1 mm, 2 mm, 3 mm, or 4 mm up to about 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1 mm, 2 mm, 3 mm, 4 mm, or 5 mm.

In any embodiment, the inner nanofiber layer of the implantable therapeutic delivery system as described herein comprises pores, said pores having a diameter of 1 nm to 50 μm. In any embodiment, the outer nanofiber layer comprises pores, said pores having a diameter of 1 nm to 50 μm. The pore diameter may, for example, range from about 1 nm; 10 nm; 20 nm; 30 nm; 40 nm; 50 nm; 60 nm; 70 nm; 80 nm; 90 nm; 100 nm; 200 nm; 300 nm; 400 nm; 500 nm; 600 nm; 700 nm; 800 nm; 900 nm; 1,000 nm; 2,000 nm; 3,000 nm; 4,000 nm; 5,000 nm; 6,000 nm; 7,000 nm; 8,000 nm; 9,000 nm; 10,000 nm; 20,000 nm; 30,000 nm; or 40,000 nm up to about 10 nm; 20 nm; 30 nm; 40 nm; 50 nm; 60 nm; 70 nm; 80 nm; 90 nm; 100 nm; 200 nm; 300 nm; 400 nm; 500 nm; 600 nm; 700 nm; 800 nm; 900 nm; 1,000 nm; 2,000 nm; 3,000 nm; 4,000 nm; 5,000 nm; 6,000 nm; 7,000 nm; 8,000 nm; 9,000 nm; 10,000 nm; 20,000 nm; 30,000 nm; 40,000 nm or 50,000 nm.

In any embodiment, the nanofiber structure of the inner nanofiber layer of the implantable therapeutic delivery system as described herein comprises a nanofiber density of <0.26 g/cm³ and the outer nanofiber layer comprises a nanofiber density of >0.26 g/cm³.

In any embodiment, the nanofiber structure of the inner nanofiber layer comprises a nanofiber density of >0.26 g/cm³ and the outer nanofiber layer comprises a nanofiber density of <0.26 g/cm³.

In any embodiment, the inner and outer nanofiber layers of the implantable therapeutic delivery system as described herein comprise pores, and the pores of the inner nanofiber layer have a greater diameter than the pores of the outer nanofiber layer.

In any embodiment, the inner and outer nanofiber layers of the implantable therapeutic delivery system as described herein comprise pores, and the pores of the outer nanofiber layer have a greater diameter than the pores of the inner nanofiber layer.

In any embodiment, the inner and outer nanofiber layers of the core substrate of the implantable therapeutic delivery system as described herein have a combined thickness of 1 μm to 5 mm. The combined thickness of the inner and outer nanofiber layers of the core of the substrate may, for example, range from about 1 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1 mm, 2 mm, 3 mm, or 4 mm to about 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1 mm, 2 mm, 3 mm, or 4 mm up to about 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1 mm, 2 mm, 3 mm, or 4 mm to about 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1 mm, 2 mm, 3 mm, 4 mm, or 5 mm.

In accordance with all aspects of the present disclosure, the nanofiber core substrate of the implantable therapeutic delivery systems as described herein has a length of 0.5 cm to 1000 m. The nanofiber core substrate may, for example, have a length ranging from about 0.5 cm, 1 cm, 10 cm, 20 cm, 30 cm, 40 cm, 50 cm, 60 cm, 70 cm, 80 cm, 90 cm, 1 m, 2 m, 3 m, 4 m, 5 m, 6 m, 7 m, 8 m, 9 m, 10 m, 20 m, 30 m, 40 m, 50 m, 60 m, 70 m, 80 m, 90 m, 100 m, 200 m, 300 m, 400 m, 500 m, 600 m, 700 m, 800 m, 900 m to 1 cm, 10 cm, 20 cm, 30 cm, 40 cm, 50 cm, 60 cm, 70 cm, 80 cm, 90 cm, 1 m, 2 m, 3 m, 4 m, 5 m, 6 m, 7 m, 8 m, 9 m, 10 m, 20 m, 30 m, 40 m, 50 m, 60 m, 70 m, 80 m, 90 m, 100 m, 200 m, 300 m, 400 m, 500 m, 600 m, 700 m, 800 m, or 900 m, up to about 1 cm, 10 cm, 20 cm, 30 cm, 40 cm, 50 cm, 60 cm, 70 cm, 80 cm, 90 cm, 1 m, 2 m, 3 m, 4 m, 5 m, 6 m, 7 m, 8 m, 9 m, 10 m, 20 m, 30 m, 40 m, 50 m, 60 m, 70 m, 80 m, 90 m, 100 m, 200 m, 300 m, 400 m, 500 m, 600 m, 700 m, 800 m, 900 m to 1 cm, 10 cm, 20 cm, 30 cm, 40 cm, 50 cm, 60 cm, 70 cm, 80 cm, 90 cm, 1 m, 2 m, 3 m, 4 m, 5 m, 6 m, 7 m, 8 m, 9 m, 10 m, 20 m, 30 m, 40 m, 50 m, 60 m, 70 m, 80 m, 90 m, 100 m, 200 m, 300 m, 400 m, 500 m, 600 m, 700 m, 800 m, 900 m or 1000 m. In some embodiment, the nanofiber core substrate has a length of 1 cm to 1 m.

In any embodiment, the nanofiber core substrate of the implantable therapeutic delivery systems as described herein comprises one or more biologically active agents selected from the group consisting of a protein, peptide, antibody or antibody fragment thereof, antibody mimetic, a nucleic acid, a small molecule, a hormone, a growth factor, an angiogenic factor, a cytokine, an anti-inflammatory agent, and combinations thereof. Suitable anti-inflammatory agents include, without limitation, diclofenac, diflunisal, etodolac, fenoprofen, flurbiprofen, ibuprofen, indomethacin, ketoprofen, ketorolac, mefenamic acid, meloxicam, nabumetone, naproxen, oxaprozin, piroxicam, salsalate, sulindac, and tolmetin.

In any embodiment, the nanofiber core substrate of the implantable therapeutic delivery systems as described herein comprises a material that is insoluble in the one or more biocompatible polymeric coatings surrounding the substrate. Suitable materials of the nanofiber core substrate include, without limitation, nylon, polyurethane, polysulfone, polyacrylonitrile, polyester such as polyethylene terephthalate and polybutester, polyvinylidene difluoride, polyacrylamide, poly (ethyl methacrylate), poly(methyl methacrylate), polyvinyl chloride, polyoxymethylene, polycarbonate, polypropylene, polyethylene, polybenzimidazole, polyaniline, polystyrene, polyvinylcarbazole, polyamide, poly vinyl phenol, cellulose acetate, polyacrylamide, poly(2-hydroxyethyl methacrylate), polyether imide, poly(ferrocenyldimethylsilane), poly(ethylene-co-vinylacetate), polyethylene-co-vinyl acetate, polyacrylic acid-polypyrene methanol, poly(ethylene-co-vinyl alcohol), polymetha-phenylene isophthalamide, poly(lactic acid), poly(ε-caprolactone), poly(lactic-co-glycolic acid), poly(l-lactide-co-ε-caprolactone), and combinations thereof.

In any embodiment, the nanofiber core substrate of the implantable therapeutic delivery systems as described herein is translucent. In any embodiment, the translucent nanofiber core substrate has >50% transmittance of light wave lengths between 400 nm and 800 nm.

In any embodiment, an elongated polymeric scaffold is positioned within the internal space of the nanofiber core substrate of the implantable therapeutic delivery systems as described herein. In any embodiment, the elongated polymeric scaffold comprises a rod, tube, or film. In any embodiment, the elongated polymeric scaffold comprises a material selected from the group consisting of silicone, PDMS, rubber, nylon, polyurethane, polysulfone, polyacrylonitrile, polyester such as polyethylene terephthalate and polybutester, polyvinylidene difluoride, polyacrylamide, poly (ethyl methacrylate), poly(methyl methacrylate), polyvinyl chloride, polyoxymethylene, polycarbonate, polypropylene, polyethylene, polybenzimidazole, polyaniline, polystyrene, polyvinylcarbazole, polyamide, poly vinyl phenol, cellulose acetate, polyacrylamide, poly(2-hydroxyethyl methacrylate), polyether imide, poly(ferrocenyldimethylsilane), poly(ethylene-co-vinylacetate), polyethylene-co-vinyl acetate, polyacrylic acid-polypyrene methanol, poly(ethylene-co-vinyl alcohol), polymetha-phenylene isophthalamide, poly(lactic acid), poly(ε-caprolactone), poly(lactic-co-glycolic acid), poly(l-lactide-co-ε-caprolactone), and combinations thereof.

In any embodiment, the elongated polymeric scaffold comprises an internal fluidic space containing an oxygen carrier. In any embodiment, the oxygen carrier comprises a perfluorinated compound. Suitable perfluorinated compounds include, without limitation, perfluorotributylamine (FC-43), perfluorodecalin, perfluorooctyl bromide, bis-perfluorobutyl-ethene, perfluoro-4-methylmorpholine, perfluorotriethylamine, perfluoro-2-ethyltetrahydrofuran, perfluoro-2-butyltetrahydrofuran, perfluoropentane, perfluoro-2-methylpentane, perfluorohexane, perfluoro-4-isopropylmorpholine, perfluorodibutyl ether, perfluoroheptane, perfluorooctane, and mixtures thereof.

In any embodiment, the elongated polymeric scaffold of the implantable therapeutic delivery systems as described herein comprises one or more therapeutic agents selected from the group consisting of therapeutic proteins, peptides, antibodies or fragments thereof, antibody mimetics, and other binding molecules, nucleic acids, small molecules, hormones, growth factors, angiogenic factors, cytokines, anti-inflammatory agents, and combinations thereof. Suitable anti-inflammatory agents are described supra.

In any embodiment, the internal space of the nanofiber core substrate is compartmentalized into two or more sub-internal spaces by one or more internal nanofiber walls.

In any embodiment, the one or more therapeutic agents positioned within the internal space of the nanofiber core substrate is selected from the group consisting of a protein, peptide, antibody or antibody fragment thereof, antibody mimetic, a nucleic acid, a small molecule, a hormone, a growth factor, an angiogenic factor, a cytokine, an anti-inflammatory agent, and combinations thereof. Suitable anti-inflammatory agents are described supra.

In any embodiment, a preparation of cells is positioned in the internal space of the nanofiber core substrate of the implantable therapeutic delivery systems as described herein, and the one or more therapeutic agents is released from said preparation of cells.

In any embodiment, one or more hydrogel films, hydrogel capsules, hydrogel fibers, or hydrogel tubes embedded with the preparation of cells is positioned in the internal space of the nanofiber core substrate.

In any embodiment, a porous scaffold coated with hydrogel comprising the preparation of cells is positioned within the internal space of the nanofiber core substrate. In any embodiment, the porous scaffold comprises a material selected from the group consisting of silicone, PDMS, rubber, nylon, polyurethane, polysulfone, polyacrylonitrile, polyester such as polyethylene terephthalate and polybutester, polyvinylidene difluoride, polyacrylamide, poly (ethyl methacrylate), poly(methyl methacrylate), polyvinyl chloride, polyoxymethylene, polycarbonate, polypropylene, polyethylene, polybenzimidazole, polyaniline, polystyrene, polyvinylcarbazole, polyamide, poly vinyl phenol, cellulose acetate, polyacrylamide, poly(2-hydroxyethyl methacrylate), polyether imide, poly(ferrocenyldimethylsilane), poly(ethylene-co-vinylacetate), polyethylene-co-vinyl acetate, polyacrylic acid-polypyrene methanol, poly(ethylene-co-vinyl alcohol), polymetha-phenylene isophthalamide, poly(lactic acid), poly(ε-caprolactone), poly(lactic-co-glycolic acid), poly(l-lactide-co-ε-caprolactone), and combinations thereof.

In any embodiment, the porous scaffold has pores having a diameter of between 1 nm and 500 μm. In any embodiment, the porous scaffold is a porous tube. In any embodiment, the porous tube comprises an internal fluidic space containing an oxygen carrier. In any embodiment, the oxygen carrier comprises a perfluorinated compound. Suitable perfluorinated compounds are described supra.

In any embodiment, the porous scaffold of the implantable therapeutic delivery systems as described herein, comprises one or more biologically active agents selected from the group consisting of a protein, peptide, antibody or antibody fragment thereof, antibody mimetic, a nucleic acid, a small molecule, a hormone, a growth factor, an angiogenic factor, a cytokine, an anti-inflammatory agent, and combinations thereof. Suitable anti-inflammatory agents are described supra.

In any embodiment, a cell growth matrix material embedded with the preparation of cells is positioned in the internal space of the nanofiber core substrate. In any embodiment, this cell growth matrix material is a hydrogel material. In any embodiment, the cell growth matrix material compromises a synthetic polymer selected from the group consisting of polyethylene glycol (PEG), poly(acrylic acid), poly(ethylene oxide), poly(vinyl alcohol), polyphosphazene, poly(hydroxyethyl methacrylate), triazole-zwitterion hydrogels, poly(sulfobetaine methacrylate), carboxybetaine methacrylate, poly[2-methacryloyloxyethyl phosphorylcholine, N-Hydroxyethyl acrylamide, copolymers thereof, derivatives thereof, and combinations thereof. In any embodiment, the cell growth matrix material compromises a natural polymeric material selected from the group consisting of collagen, elastin, fibrin, gelatin, gelatin-methacryloyl, silk fibroin, glycosaminoglycans, dextran, alginate, agarose, chitosan, bacterial cellulose, keratin, matrigel, decellularized hydrogels, and derivatives or combinations thereof.

In any embodiment, the cell growth matrix material further comprises one or more cell factors to enhance cell growth, differentiation, and/or survival selected from the group consisting of glutamine, non-essential amino acids, epidermal growth factors, fibroblast growth factors, transforming growth factor/bone morphogenetic proteins, platelet derived growth factors, insulin growth factors, cytokines, fibronectin, laminin, heparin, collagen, glycosaminoglycan, proteoglycan, elastin, chitin derivatives, fibrin, and fibrinogen, FGF, bFGF, acid FGF (aFGF), FGF-2, FGF-4, EGF, PDGF, TGF-beta, angiopoietin-1, angiopoietin-2, placental growth factor (PlGF), VEGF, PMA (phorbol 12-myristate 13-acetate), combinations thereof.

In any embodiment, the preparation of cells positioned in the internal space of the nanofiber core substrate of the implantable therapeutic delivery systems as described herein, is a preparation of single cells or a preparation of cell aggregates. In any embodiment, the preparation of cells is a preparation of primary cells or a preparation of immortalized cells. In any embodiment, the preparation of cells is a preparation of mammalian cells. In any embodiment, the preparation of cells is selected from the group consisting of a preparation of primate cells, rodent cells, canine cells, feline cells, equine cells, bovine cells, and porcine cells. In any embodiment, the preparation of cells is a preparation of human cells. In any embodiment, the preparation of cells is a preparation of stem cells or stem cell derived cells. In any embodiment, the stem cells are pluripotent, multipotent, oligopotent, or unipotent stem cells. In any embodiment, the preparation of stem cells is selected from the group consisting of embryonic stem cells, epiblast cells, primitive ectoderm cells, primordial germ cells, and induced pluripotent stem cells. In any embodiment, the preparation of cells is a preparation of cells selected from the group consisting of smooth muscle cells, cardiac myocytes, platelets, epithelial cells, endothelial cells, urothelial cells, fibroblasts, embryonic fibroblasts, myoblasts, chondrocytes, chondroblasts, osteoblasts, osteoclasts, keratinocytes, hepatocytes, bile duct cells, islet cells, thyroid, parathyroid, adrenal, hypothalamic, pituitary, ovarian, testicular, salivary gland cells, adipocytes, embryonic stem cells, mesenchymal stem cells, neural cells, endothelial progenitor cells, hematopoietic cells, precursor cells, mesenchymal stromal cells, Baby Hamster Kidney (BHK) cells, Chinese Hamster Ovary cells, Human Amniotic Epithelial (HAE) cells, choroid plexus cells, chromaffin cells, adrenal chromaffin cells, pheochomocytoma cell line PC12, human retinal pigment epithelium cells, recombinant human retinal pigment epithelium cells, NGF-secreting Baby Hamster Kidney (BHK) cells, human bone marrow-derived stem cells transfected with GLP-1, BDNF-producing fibroblasts, NGF-producing cells, CNTF-producing cells, BDNF-secreting Schwann cells, IL-2-secreting myoblasts, endostatin-secreting cells, and cytochrome P450 enzyme overexpressed feline kidney epithelial cells, myogenic cells, embryonic stem cell-derived neural progenitor cells, irradiated tumor cells, proximal tubule cells, neural precursor cells, astrocytes, genetically engineered cells.

In any embodiment, the preparation of cells comprises a cell density of between 1×10³ to 1×10¹⁰ cells/mL. For example, the cell density may range from about 1×10³ cells/mL, 1×10⁴ cells/mL, 1×10⁵ cells/mL, 1×10⁶ cells/mL, 1×10⁷ cells/mL, 1×10⁸ cells/mL, or 1×10⁹ cells/mL up to about 1×10⁴ cells/mL, 1×10⁵ cells/mL, 1×10⁶ cells/mL, 1×10⁷ cells/mL, 1×10⁸ cells/mL, 1×10⁹ cells/mL or 1×10¹⁰ cells/mL.

In any embodiment, the preparation of cells is a preparation comprising islet cells that release insulin and glucagon. In any embodiment, the preparation comprising islet cells is a preparation of human cells, porcine cells, or rodent cells. In any embodiment, the preparation of cells comprises an islet density between 1×10³ to 6×10⁵ islet equivalents (IEQs)/mL. For example, the islet equivalents may range from about 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, or 5×10⁵ up to about 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, or 6×10⁵.

In any embodiment, the proximal and distal ends of the nanofiber core substrate of the implantable therapeutic delivery systems as described herein are sealed. In any embodiment, the proximal and distal ends of the nanofiber core substrate are sealed by a heat seal, a suture knot, a clamp, a rubber seal, or a screw closure.

In any embodiment, the outer biocompatible polymeric coating of the implantable therapeutic delivery systems as described herein is a hydrogel material. In any embodiment, the hydrogel material is a synthetic polymer selected from the group consisting of polyethylene glycol (PEG), poly(acrylic acid), poly(ethylene oxide), poly(vinyl alcohol), polyphosphazene, poly(hydroxyethyl methacrylate), triazole-zwitterion hydrogels (TR-qCB, TR-CB, TR-SB), poly(sulfobetaine methacrylate), carboxybetaine methacrylate, poly[2-methacryloyloxyethyl phosphorylcholine, N-hydroxyethyl acrylamide, a copolymer thereof, a derivatives thereof, and a combination thereof. In any embodiment, the hydrogel material is a natural polymeric material selected from the group consisting of collagen, elastin, fibrin, gelatin, gelatin-methacryloyl, silk fibroin, glycosaminoglycans, dextran, alginate, agarose, chitosan, bacterial cellulose, keratin, matrigel, decellularized hydrogels, derivatives thereof, and combinations thereof. In any embodiment, the hydrogel material is a zwitterionically modified hydrogel. Suitable zwitterionically modified hydrogels include those described in Liu et al., “Developing mechanically robust, triazole-zwitterionic hydrogels to mitigate foreign body response (FBR) for islet encapsulation,” Biomaterials, 230:119640 (2019); Liu et al., “Zwitterionically Modified Alginates Mitigate Cellular Overgrowth for Cell Encapsulation,” Nat. Commun. 10(1):5262 (2019); and U.S. Patent Application Publication No. 20190389979 to Ma and Liu, which are hereby incorporated by reference in their entirety.

In any embodiment, the hydrogel material comprises a pure alginate, a modified alginate, or a mixture of pure and modified alginate. In any embodiment, the modified alginate is a zwitterionically modified alginate. Suitable zwitterionically modified alginates include, without limitation, those disclosed in Liu et al., “Zwitterionically Modified Alginates Mitigate Cellular Overgrowth for Cell Encapsulation,” Nat. Commun. 10(1):5262 (2019) and U.S. Patent Application Publication No. 20190389979 to Ma and Liu, which are hereby incorporated by reference in their entirety. In any embodiment, the hydrogel material comprises a mixture of pure alginate and modified alginate in a ratio of about 1:1000 to 1000:1 (v/v). The ratio of the pure alginate and modified alginate may range from about 1:1000; 10:1000; 20:1000; 30:1000; 40:1000; 50:1000; 60:1000; 70:1000; 80:1000; 90:1000; 100:1000; 200:1000; 300:1000; 400:1000; 500:1000; 600:1000; 700:1000; 800:1000; 900:1000; or 1,000:1000 (1:1) up to about 1000:1; 1000:10; 1000:20; 1000:30; 1000:40; 1000:50; 1000:60; 1000:70; 1000:80; 1000:90; 1000:100; 1000:200; 1000:300; 1000:400; 1000:500; 1000:600; 1000:700; 1000:800; or 1000:900. In any embodiment, the hydrogel material comprises a mixture of pure alginate and modified alginate in a ratio of about 3:7 to 7:3 (v/v). The ratio of pure alginate to modified alginate may, for example, be about 3:7, 4:6, 5:5 (1:1), 6:4, or 7:3 (v/v).

In any embodiment, the biocompatible polymeric coating surrounding the nanofiber core substrate has a thickness of 1 nm to 5 mm, wherein the standard deviation in thickness of the polymeric coating around the entirety of the nanofiber core substrate is <100%. The biocompatible polymeric coating may, for example, have a thickness ranging from about 1 nm; 10 nm; 100 nm; 200 nm; 300 nm; 400 nm; 500 nm; 600 nm; 700 nm; 800 nm; 900 nm; 1.00 nm; 2,000 nm; 3,000 nm; 4,000 nm; 5,000 nm; 6,000 nm; 7,000 nm; 8,000 nm; 9,000 nm; 10,000 nm; 20,000 nm; 30,000 nm; 40,000 nm; 50,000 nm; 60,000 nm; 70,000 nm; 80,000 nm; 90,000 nm; 100,000 nm; 200,000 nm; 300,000 nm; 400,000 nm; 500,000 nm; 600,000 nm; 700,000 nm; 800,000 nm; 900,000 nm; 1 mm; 2 mm; 3 mm; or 4 mm up to about 10 nm; 100 nm; 200 nm; 300 nm; 400 nm; 500 nm; 600 nm; 700 nm; 800 nm; 900 nm; 1.00 nm; 2,000 nm; 3,000 nm; 4,000 nm; 5,000 nm; 6,000 nm; 7,000 nm; 8,000 nm; 9,000 nm; 10,000 nm; 20,000 nm; 30,000 nm; 40,000 nm; 50,000 nm; 60,000 nm; 70,000 nm; 80,000 nm; 90,000 nm; 100,000 nm; 200,000 nm; 300,000 nm; 400,000 nm; 500,000 nm; 600,000 nm; 700,000 nm; 800,000 nm; 900,000 nm; 1 mm; 2 mm; 3 mm; 4 mm; or 5 mm.

In any embodiment, the biocompatible polymeric coating of the implantable therapeutic delivery systems as described herein is crosslinked and interlocked to the nanofiber core substrate.

In any embodiment, the biocompatible polymeric coating of the implantable therapeutic delivery systems as described herein, comprises one or more biologically active agents selected from the group consisting of a protein, peptide, antibody or antibody fragment thereof, antibody mimetic, a nucleic acid, a small molecule, a hormone, a growth factor, an angiogenic factor, a cytokine, an anti-inflammatory agent, and combinations thereof. Suitable anti-inflammatory agents are described supra.

In any embodiment, the implantable system described herein comprises one or more contrast agents to facilitate in vivo monitoring of implant placement, location of implant at some time point after implantation, health of the implant, deleterious effects on non-target cell types, inflammation, and/or fibrosis. Suitable contrast agents include, without limitation, nanoparticles, nanocrystals, gadolinium, iron oxide, iron platinum, manganese, iodine, barium, microbubbles, fluorescent dyes, and others known to those of skill in the art.

Methods of in vivo monitoring include but are not limited to confocal microscopy, 2-photon microscopy, high frequency ultrasound, optical coherence tomography (OCT), photoacoustic tomography (PAT), computed tomography (CT), magnetic resonance imaging (MRI), single photon emission computed tomography (SPECT), and positron emission tomography (PET). These alone or combined can provide useful means to monitoring the implantable system.

Another aspect of the present disclosure is directed to a method of delivering a therapeutic agent to a subject in need thereof. This method involves implanting any one of the implantable therapeutic delivery systems as described herein into the subject.

In some embodiments, the subject in need of treatment thereof, is a subject having diabetes, and the method of delivering a therapeutic agent to the subject involves implanting an implantable therapeutic delivery system into the subject having diabetes. In accordance with this embodiment, the one or more therapeutic agents of the implantable therapeutic delivery system is insulin, glucagon, or a combination thereof. In any embodiment, the insulin, glucagon, or combination thereof is released from a preparation of cells positioned in the internal space of the nanofiber core substrate. In any embodiment, the preparation of cells comprises a preparation of islets. In any embodiment, the preparation of islets is a preparation of primate islets, rodent islets, canine islets, feline islets, equine islets, bovine islets, or porcine islets. In any embodiment, the preparation of islets is derived from a preparation of stem cells. In any embodiment, the preparation of stem cells is a preparation of pluripotent, multipotent, oligopotent, or unipotent stem cells. In any embodiment, the preparation of stem cells is a preparation comprising embryonic stem cells, epiblast cells, primitive ectoderm cells, primordial germ cells, and induced pluripotent stem cells.

In some embodiments, the subject in need of treatment thereof is a subject having a bleeding disorder, and the method of delivering a therapeutic agent to the subject involves implanting an implantable therapeutic delivery system as described herein into the subject having the bleeding disorder. In accordance with this embodiment, the bleeding disorder can be any bleeding disorder, such as hemophilia A, hemophilia B, von Willebrand disease, Factor I deficiency, Factor II deficiency, Factor V deficiency, Factor VII deficiency, Factor X deficiency, Factor XI deficiency, Factor XII deficiency, and Factor XIII deficiency. In any embodiment, the one or more therapeutic agents is a blood clotting factor released from a preparation of cells positioned in the internal space of the nanofiber core substrate. In any embodiment, the preparation of cells comprises recombinant myoblasts, mesenchymal stromal cells, induced pluripotent stem cell derived endothelial cells, or a combination thereof. In any embodiment, the blood clotting factor is selected from the group consisting of Factor I, Factor II, Factor V, Factor VII, Factor VIII, Factor IX, Factor X, Factor XI, Factor XII, Factor XIII, and combinations thereof.

In some embodiments, the subject in need of treatment thereof is a subject having a lysosomal storage disorder, and the method of delivering a therapeutic agent to the subject involves implanting an implantable therapeutic delivery system as described herein into the subject having the lysosomal storage disorder. In any embodiment, the one or more therapeutic agents is an enzyme released from a preparation of cells positioned in the internal space of the nanofiber core substrate. In any embodiment, the preparation of cells comprises hematopoietic stem cells, fibroblasts, myoblasts, Baby Hamster Kidney (BHK) cells, Chinese Hamster Ovary cells, Human Amniotic Epithelial (HAE) cells, or combinations thereof. In any embodiment, the enzyme is selected from the group consisting of α-L-iduronidase, Iduronate-2-sulfatase, α-glucuronidase, Arylsulfatase A, alpha-Galactosidase A, and combinations thereof.

In some embodiments, the subject in need of treatment thereof is a subject having a neurological disorder, and the method of delivering a therapeutic agent to the subject involves implanting an implantable therapeutic delivery system as described herein into the subject having the neurological disorder. In any embodiment, neurological disorder is a sensory disorder. In any embodiment, the neurological disorder is selected from the group consisting of Parkinson's disorder, Alzheimer's disease, epilepsy, Huntington's disease, Amyotrophic lateral sclerosis, chronic pain, visual and hearing loss. In any embodiment, the one or more therapeutic agents is a therapeutic molecule released from a preparation of cells positioned in the internal space of the nanofiber core substrate. In any embodiment, the preparation of cells comprises choroid plexus cells, chromaffin cells, pheochomocytoma cell line PC12, human retinal pigment epithelial cells, NGF-secreting Baby Hamster Kidney (BHK) cells, myoblasts, human bone marrow-derived stem cells transfected with GLP-1, BDNF-producing fibroblasts, NGF-producing cells, CNTF-producing cells, adrenal chromaffin cells, BDNF-secreting Schwann cells, and combinations thereof. In any embodiment, the therapeutic molecule is selected from the group consisting of cerebrospinal fluid, extracellular fluid, levodopa, nerve growth factor (NGF), ciliary neurotrophic factor (CNTF), BLP-1, brain-derived neurotrophic factor (BDNF), vascular endothelial growth factor (VEGF), enkephalin, adrenaline, catecholamine, and combinations thereof.

In some embodiments, the subject in need of treatment thereof is a subject having cancer, and the method of delivering a therapeutic agent to the subject involves implanting an implantable therapeutic delivery system as described herein into the subject having cancer disorder. In any embodiment, the one or more therapeutic agents is a therapeutic molecule released from a preparation of cells positioned in the internal space of the nanofiber core substrate. In any embodiment, the preparation of cells comprises IL-2-secreting myoblasts, endostatin-secreting cells, Chinese Hamster Ovary cells, and cytochrome P450 enzyme overexpressed feline kidney epithelial cells. In any embodiment, the therapeutic molecule is selected from IL-2, endostatin, cytochrome P450 enzyme, and combinations thereof.

In some embodiments, the subject in need of treatment thereof is a subject having chronic eye disease and the method of delivering a therapeutic agent to the subject involves implanting an implantable therapeutic delivery system as described herein into the subject having a chronic eye disease. In any embodiment, this method further involves administering one or more trophic factors to the subject to protect compromised retinal neurons and to restore neural circuits. In any embodiment, the chronic eye disease is selected from the group consisting of age-related macular degeneration, diabetic retinopathy, retinitis pigmentosa, glaucoma, macular telangiectasia, and combinations thereof. In any embodiment, the one or more therapeutic agents is a therapeutic molecule released from a preparation of cells positioned in the internal space of the nanofiber core substrate. In any embodiment, the preparation of cells comprises human retinal pigment epithelium cells, recombinant human retinal pigment epithelium cells, or a combination thereof. In any embodiment, the therapeutic molecule is selected from the group consisting of ciliary neurotrophic factor, antagonists against vascular endothelial growth factor and platelet-derived growth factor, and combinations thereof.

In some embodiments, the subject in need of treatment thereof is a subject having kidney failure and the method of delivering a therapeutic agent to the subject involves implanting an implantable therapeutic delivery system as described herein into the subject having kidney failure. In any embodiment, the one or more therapeutic agents is a therapeutic molecule released from a preparation of cells positioned in the internal space of the nanofiber core substrate. In any embodiment, the preparation of cells comprises renal proximal tubule cells, mesenchymal stem cells, and a combination thereof.

In some embodiments, the subject in need of treatment thereof is a subject having chronic pain and the method of delivering a therapeutic agent to the subject involves implanting an implantable therapeutic delivery system as described herein into the subject having chronic pain. In any embodiment, chronic pain is chronic pain caused by degenerative back and knee, neuropathic back and knee, or cancer. In any embodiment, the one or more therapeutic agents is a therapeutic molecule released from a preparation of cells positioned in the internal space of the nanofiber core substrate. In any embodiment, the preparation of cells comprises chromaffin cells, neural precursor cells, mesenchymal stem cells, astrocytes, and genetically engineered cells, or a combination thereof. In any embodiment, the therapeutic molecule is selected from the group consisting of catecholamine, opioid peptides, enkephalins, and combinations thereof.

In some embodiments, the method of delivering a therapeutic agent to a subject in need thereof involves implanting an implantable therapeutic delivery system as described herein using a laparoscopic procedure. In some embodiments, the therapeutic delivery system is implanted intraperitoneally, percutaneously, or subcutaneously. In some embodiments, implanting the therapeutic delivery system involves suturing the delivery system to a body wall of the subject. In some embodiments, implanting the therapeutic delivery system involves anchoring the delivery system to a body wall of the subject via a transabdominal portal. In some embodiments, implanting the therapeutic delivery system involves wrapping the delivery system in omentum of the subject. In some embodiments, implanting the therapeutic delivery system involves positioning the delivery system in a cavity between the liver and the diaphragm. In some embodiments, implanting the therapeutic delivery system involves anchoring the delivery system to the diaphragm. In some embodiments, the method of delivering a therapeutic agent to a subject in need thereof further comprises retrieving the implantable therapeutic delivery system from the subject. In some embodiments, the method of delivering a therapeutic agent further involves implanting a replacement implantable therapeutic delivery system after said retrieving.

Another aspect of the present disclosure relates to method of making a nanofiber core substrate of any of the implantable therapeutic delivery systems as described herein. This method involves providing one or more polymer solutions comprising 1% to 50% polymer in a solvent; electrospinning said one or more polymer solutions onto a rotating collecting rod, wherein said collecting rod is coated with a viscous saccharide solution, to form the nanofiber core substrate; and removing the porous nanofiber core substrate from the collecting rod.

In any embodiment, the one or more polymer solutions are electrospun using a single channel nozzle or a multi-channel nozzle comprising needles of different diameters.

In any embodiment, removing the porous nanofiber core substrate from the collecting rod involves dissolving the saccharide solution from the collecting rod in water.

In any embodiment of this method, the solvent comprises hexafluoroisopropanol (HFIP). In any embodiment, the solvent is pure HFIP. In any embodiment, the solvent comprises a mixture of HFIP and formic acid. Other suitable organic solvents include, for example, and without limitation, dichloromethane, N,N-dimethyl formamide, ethanol, methanol, or any combination thereof.

In any embodiment of this method, the polymer solution is a 1% to 50% polymer solution, i.e., the polymer comprises about 1%, 5%, 10%, 15%, 20%, 25%, 30%, 35%, 40%, 45% or 50% of the solution. In any embodiment of this method, the polymer solution comprises one or more polymers selected from the group consisting of nylon, polyurethane, polysulfone, polyacrylonitrile, polyester such as polyethylene terephthalate and polybutester, polyvinylidene difluoride, polyacrylamide, poly (ethyl methacrylate), poly(methyl methacrylate), polyvinyl chloride, polyoxymethylene, polycarbonate, polypropylene, polyethylene, polybenzimidazole, polyaniline, polystyrene, polyvinylcarbazole, polyamide, poly vinyl phenol, cellulose acetate, polyacrylamide, poly(2-hydroxyethyl methacrylate), polyether imide, poly(ferrocenyldimethylsilane), poly(ethylene-co-vinylacetate), polyethylene-co-vinyl acetate, polyacrylic acid-polypyrene methanol, poly(ethylene-co-vinyl alcohol), polymetha-phenylene isophthalamide, poly(lactic acid), poly(ε-caprolactone), poly(lactic-co-glycolic acid), and poly(l-lactide-co-ε-caprolactone).

In any embodiment of this method, the viscous saccharide solution comprises one or more monosaccharides, disaccharides, oligosaccharides, and mixtures thereof. In any embodiment, the saccharide is selected from glucose, galactose, fructose, sucrose, lactose, maltose, trehalose, and mixtures thereof. In any embodiment, the saccharide solution has a viscosity of >4×10³ mPa·s. In any embodiment, the viscous saccharide solution is a sucrose solution comprising about 25 g/mL sucrose.

In any embodiment, the method of the method of making a nanofiber core substrate of any of the implantable therapeutic delivery systems as described herein further involves applying a solvent vapor to the collecting rod during said electrospinning under conditions effective to generate a translucent porous nanofiber substrate.

Another aspect of the present disclosure relates a method of producing an implantable therapeutic delivery system. This method involves providing a longitudinally extending nanofiber core substrate, said substrate having a proximal and a distal end, each proximal and distal end having an opening to at least one internal space within the nanofiber core substrate; sealing the proximal end of the nanofiber core substrate; soaking the sealed proximal end and outer surface of the nanofiber core substrate in a biocompatible polymer solution to allow polymer solution penetration into the nanofiber core substrate; filling the at least one internal space of the nanofiber core substrate with one or more crosslinking agents to crosslink the coated biocompatible polymer solution to the nanofiber core substrate; loading one or more therapeutic agents into the at least one internal space of the nanofiber core substrate through the opening at the distal end of the nanofiber core substrate; sealing the distal end of the loaded nanofiber core substrate; and coating the sealed distal end of the nanofiber core substrate with the biocompatible polymer solution to form the implantable therapeutic delivery system.

Another aspect of the present disclosure relates to a method of producing an implantable therapeutic delivery system. This method comprises: providing a longitudinally extending nanofiber core substrate, said substrate having a proximal and a distal end, each proximal and distal end having an opening to at least one internal space within the nanofiber core substrate; sealing the proximal end of the nanofiber core substrate; applying a biocompatible polymer solution to the sealed proximal end and outer surface of the nanofiber core substrate; loading one or more therapeutic agents into the at least one internal space of the nanofiber core substrate through the opening at the distal end of the nanofiber core substrate; sealing the distal end of the loaded nanofiber core substrate; applying the biocompatible polymer solution to the sealed distal end of the nanofiber core substrate; and crosslinking the coated biocompatible polymer solution to the nanofiber core substrate to form the implantable therapeutic delivery system.

Another aspect of the present disclosure relates to a method of producing an implantable therapeutic delivery system. This method involves providing a longitudinally extending nanofiber core substrate, said substrate having a proximal and a distal end, each proximal and distal end having an opening to at least one internal space within the nanofiber core substrate; sealing the proximal end of the nanofiber core substrate; loading one or more therapeutic agents into the at least one internal space of the nanofiber core substrate through the opening at the distal end of the nanofiber core substrate; sealing the distal end of the loaded nanofiber core substrate; soaking the sealed and loaded nanofiber core substrate in a cross-linker solution; coating the cross-linker soaked nanofiber core substrate with a biocompatible polymer solution; and crosslinking the coated biocompatible polymer solution to the nanofiber core substrate to form the implantable therapeutic delivery system.

In any embodiment, the step of loading one or more therapeutic agents into the at least one internal space of the nanofiber core substrate in accordance with the above described methods involves positioning one or more hydrogel films, hydrogel capsules, hydrogel fibers, hydrogel tubes, or a combination thereof within the at least one internal space, wherein said one or more films, capsules, fibers or tubes are embedded with a preparation of cells that release the one or more therapeutic agents.

In any embodiment, the step of loading one or more therapeutic agents into the at least one internal space of the nanofiber core substrate in accordance with the above described methods involves providing a porous scaffold coated with an outer layer of hydrogel, said hydrogel embedded with a preparation of cells that release the one or more therapeutic agents, and positioning the porous scaffold coated with hydrogel embedded cells within the at least one internal space of the nanofiber core substrate.

In any embodiment, the step of loading one or more therapeutic agents into the at least one internal space of the nanofiber core substrate in accordance with the above described methods involves providing a mixture of extracellular matrix precursor material and cells; loading said mixture into the at least one internal space through the distal end of the nanofiber core substrate, and crosslinking the extracellular matrix material.

In any embodiment, the step of sealing the proximal and/or distal end of the nanofiber core substrate is carried out using a thermo sealer.

In any embodiment, the one or more outer biocompatible polymeric solutions that is applied or coated on the nanofiber core substrate is a hydrogel material. In any embodiment, the hydrogel material is a synthetic polymer selected from the group consisting polyethylene glycol (PEG), poly(acrylic acid), poly(ethylene oxide), poly(vinyl alcohol), polyphosphazene, poly(hydroxyethyl methacrylate), triazole-zwitterion hydrogels, poly(sulfobetaine methacrylate), carboxybetaine methacrylate, poly[2-methacryloyloxyethyl phosphorylcholine, N-Hydroxyethyl acrylamide, copolymers thereof, derivatives thereof, and combinations thereof. In any embodiment, the hydrogel material is a natural polymeric material selected from the group consisting of collagen, elastin, fibrin, gelatin, gelatin-methacryloyl, silk fibroin, glycosaminoglycans, dextran, alginate, agarose, chitosan, bacterial cellulose, keratin, matrigel, decellularized hydrogels, derivatives thereof, and combinations thereof. In any embodiment, the hydrogel material is a zwitterionically modified hydrogel such as the zwitterionically modified hydrogels described in Liu et al. “Developing mechanically robust, triazole-zwitterionic hydrogels to mitigate foreign body response (FBR) for islet encapsulation,” Biomaterials, 230:119640 (2019); Liu et al., “Zwitterionically Modified Alginates Mitigate Cellular Overgrowth for Cell Encapsulation,” Nat. Commun. 10(1):5262 (2019); and U.S. Patent Application Publication No. 20190389979 to Ma and Liu, which are hereby incorporated by reference in their entirety. In any embodiment, the hydrogel material comprises a pure alginate, a modified alginate, or a mixture of pure and modified alginate. In any embodiment, the modified alginate is a zwitterionically modified alginate as described in Liu et al., “Zwitterionically Modified Alginates Mitigate Cellular Overgrowth for Cell Encapsulation,” Nat. Commun. 10(1):5262 (2019); and U.S. Patent Application Publication No. 20190389979 to Ma and Liu, which are hereby incorporated by reference in their entirety. In any embodiment, the hydrogel material comprises a mixture of pure alginate and modified alginate in a ratio of about 1:1000 to 1000:1 (v/v). The ratio of the pure alginate and modified alginate may range from about 1:1000; 10:1000; 20:1000; 30:1000; 40:1000; 50:1000; 60:1000; 70:1000; 80:1000; 90:1000; 100:1000; 200:1000; 300:1000; 400:1000; 500:1000; 600:1000; 700:1000; 800:1000; 900:1000; or 1,000:1000 (1:1) up to about 1000:1; 1000:10; 1000:20; 1000:30; 1000:40; 1000:50; 1000:60; 1000:70; 1000:80; 1000:90; 1000:100; 1000:200; 1000:300; 1000:400; 1000:500; 1000:600; 1000:700; 1000:800; or 1000:900. In any embodiment, the hydrogel material comprises a mixture of pure alginate and modified alginate in a ratio of about 3:7 to 7:3 (v/v). The ratio of pure alginate to modified alginate may, for example, be about 3:7, 4:6, 5:5 (1:1), 6:4, or 7:3 (v/v).

In any embodiment, the step of crosslinking the coated biocompatible polymer solution to the nanofiber core substrate involves exposing the biocompatible polymer solution to one or more crosslinking agents. In any embodiment, the one or more crosslinking agents is a cation selected from Ba²⁺, Ca²⁺, Cd²⁺, Cu²⁺, Fe²⁺, Mg²⁺, Mn²⁺, Ni²⁺, Pb²⁺, Sn²⁺, Sr²⁺, and Zn²⁺.

Another aspect of the present disclosure is directed to a method of producing a porous nanofiber substrate. This method involves providing one or more polymer-solvent solutions; coating a rotating collecting rod with a viscous saccharide solution; electrospinning said one or more polymer solutions onto the coated rotating collecting rod to form the porous nanofiber substrate; and dissolving the viscous saccharide solution from the collecting rod, thereby removing the porous nanofiber substrate from the collecting rod.

In any embodiment of this method, the viscous saccharide solution comprises one or more monosaccharides, disaccharides, oligosaccharides, and mixtures thereof. In any embodiment, the saccharide is selected from glucose, galactose, fructose, sucrose, lactose, maltose, trehalose, and mixtures thereof. In any embodiment, the saccharide solution has a viscosity of >4×10³ mPa·s. In any embodiment, the viscous saccharide solution is a sucrose solution comprising about 25 g/mL sucrose.

In any embodiment of this method, the solvent comprises hexafluoroisopropanol (HFIP). Other suitable organic solvents include, for example, and without limitation, dichloromethane, N,N-dimethyl formamide, ethanol, methanol, or any combination thereof.

In any embodiment of this method, the polymer solution is a 1% to 50% polymer solution, i.e., the polymer comprises about 1%, 5%, 10%, 15%, 20%, 25%, 30%, 35%, 40%, 45% or 50% of the solution. Suitable polymer solution may comprise one or more polymers selected from the group consisting of nylon, polyurethane, polysulfone, polyacrylonitrile, polyester such as polyethylene terephthalate and polybutester, polyvinylidene difluoride, polyacrylamide, poly (ethyl methacrylate), poly(methyl methacrylate), polyvinyl chloride, polyoxymethylene, polycarbonate, polypropylene, polyethylene, polybenzimidazole, polyaniline, polystyrene, polyvinylcarbazole, polyamide, poly vinyl phenol, cellulose acetate, polyacrylamide, poly(2-hydroxyethyl methacrylate), polyether imide, poly(ferrocenyldimethylsilane), poly(ethylene-co-vinylacetate), polyethylene-co-vinyl acetate, polyacrylic acid-polypyrene methanol, poly(ethylene-co-vinyl alcohol), polymetha-phenylene isophthalamide, poly(lactic acid), poly(ε-caprolactone), poly(lactic-co-glycolic acid), and poly(l-lactide-co-ε-caprolactone).

Another aspect of the present disclosure is directed to a thermo sealing device. This thermos sealing device comprises a first substrate portion comprising a cut-out along its peripheral edge; a second substrate portion comprising a cut-out that is substantially identical in shape and size to the cut-out of the first substrate, said second substrate further comprising a trench configured to house a heating element, wherein said trench aligns with the cut-out of the second substrate; a connector connecting the first and second substrate portions in a manner that aligns the cut-out of the first substrate portion with the cut-out of the second substrate portion; and a heating element positioned in the trench of the second substrate portion.

In any embodiment, the heating element is a ribbon-like structure. In any embodiment, the heating element is flat. In any embodiment, the heating element is positioned in the trench standing on its narrow edge.

In any embodiment, the first and second substrate portions of the thermos sealing device are made from a separate pieces of substrate material. In any embodiment, the first and second substrate portions of the thermos sealing device are made from a single piece of substrate material. In any embodiment, the first and second substrate portions of the thermo sealing device are comprised of a heat resistant material. Suitable heat resistant materials include, without limitation, ceramics and metals. In any embodiment, the first and second substrate portions of the thermos sealing device are comprised of a thermosetting material. Suitable thermosetting materials include, without limitation, polydimethylsiloxanes, epoxy resins, melamine formaldehydes, polyester resins, urea formaldehydes, and phenol formaldehydes.

In any embodiment, the thermosetting material is a transparent material.

In any embodiment, the cut-out of the first and second substrate portions has a rounded edge. In any embodiment, the cut-out of the first and second substrate portions has a straight edge. In any embodiment, the cut-out of the first and second substrate portions is adapted to a shape selected from an arch, triangle, square, circle, etc.

Encapsulation and transplantation of insulin-producing cells offer a promising curative treatment for type 1 diabetes (T1D). However, biomaterials used to encapsulate cells often elicit foreign body responses, leading to cellular overgrowth and deposition of fibrotic tissue, which in turn diminishes mass transfer to and from transplanted cells. The encapsulation device must be safe, ideally retrievable, and scalable to meet clinical requirements. Here we report a durable and safe nanofibrous device coated with a thin and uniform, fibrosis-mitigating zwitterionically modified alginate hydrogel for encapsulation of islets and stem cell-derived beta (SC-β) cells. We designed the device with a configuration that has cells encapsulated within a cylindrical wall allowing scale-up in both radial and longitudinal directions without sacrificing mass transfer. Due to its facile mass transfer and low level of fibrotic reaction, the device supports long-term cell engraftment, correcting diabetes in C57BL6/J mice with rat islets for up to 399 days and SCID-beige mice with human SC-β cells for up to 238 days. We further demonstrated the scalability and retrievability in dogs. These results demonstrate the potential of this new device for cell therapies for T1D and other diseases.

Preferences and options for a given aspect, feature, embodiment, or parameter of the technology described herein should, unless the context indicates otherwise, be regarded as having been disclosed in combination with any and all preferences and options for all other aspects, features, embodiments, and parameters of the technology.

The following Examples are presented to illustrate various aspects of the present application, but are not intended to limit the scope of the claimed application.

### EMBODIMENTS OF THE DISCLOSURE

Embodiment 1 is an implantable therapeutic delivery system comprising: a nanofiber core substrate having proximal and distal ends, said nanofiber core substrate having an interior nanofiber wall defining an internal space extending longitudinally along the core substrate, with one or more therapeutic agents positioned within the internal space; and a hydrogel surrounding said nanofiber core substrate, wherein said hydrogel comprises 0.1% to 20% of an alginate mixture, said alginate mixture comprising zwitterionically modified alginate and pure alginate in a ratio of 1:1000 to 1000:1 (v/v).

Embodiment 2 is an implantable therapeutic delivery system comprising: a nanofiber core substrate having proximal and distal ends, said nanofiber core substrate having an interior nanofiber wall defining an internal space extending longitudinally along the core substrate, with one or more therapeutic agents positioned within the internal space; and a biocompatible polymeric coating surrounding said nanofiber core substrate, wherein said biocompatible polymeric coating has a thickness of 1 nm to 5 mm, and wherein the standard deviation in thickness of the polymeric coating around the entirety of the nanofiber core substrate is <100%.

Embodiment 3 is the implantable therapeutic delivery system of Embodiment 2, wherein the standard deviation in thickness of the polymeric coating around the entirety of the nanofiber core substrate is <60%.

Embodiment 4 is the implantable therapeutic delivery system of Embodiment 1, wherein the hydrogel comprises 1% to 4% of an alginate mixture and/or wherein the alginate mixture comprises zwitterionically modified alginate and pure alginate in a ratio of 7:3 to 3:7 (v/v).

Embodiment 5 is the implantable therapeutic delivery system of Embodiment 1 or Embodiment 2, wherein the interior nanofiber wall of the nanofiber core substrate forms a tube having a diameter of 0.1 mm to 30 cm.

Embodiment 6 is the implantable therapeutic delivery system of Embodiment 5, wherein the tube is a conical tube.

Embodiment 7 is the implantable therapeutic delivery system of Embodiment 5, wherein the tube is a cylindrical tube.

Embodiment 8 is the implantable therapeutic delivery system of any one of Embodiments 1-7, wherein the interior wall has a thickness of 1 μm to 5 mm.

Embodiment 9 is the implantable therapeutic delivery system of any one of Embodiments 1-8, wherein the nanofiber core substrate has a nanofiber density of 0.01 g/cm³ to 1.5 g/cm³.

Embodiment 10 is the implantable therapeutic delivery system of any one of Embodiments 1-9, wherein nanofibers of the nanofiber core substrate have a diameter of 1 nm to 50 μm.

Embodiment 11 is the implantable therapeutic delivery system of any one of Embodiments 1-10, wherein the nanofiber core substrate comprises pores, said pores having a diameter of 1 nm to 50 μm.

Embodiment 12 is the implantable therapeutic delivery system of any one of Embodiments 1-11, wherein nanofiber composition of the nanofiber core substrate is homogeneous.

Embodiment 13 is the implantable therapeutic delivery system of any one of Embodiments 1-11, wherein nanofiber composition of the nanofiber core substrate is heterogeneous.

Embodiment 14 is an implantable therapeutic delivery system comprising: a nanofiber core substrate having proximal and distal ends, said nanofiber core substrate defined by an inner nanofiber layer and an outer nanofiber layer surrounding the inner nanofiber layer, wherein the inner nanofiber layer has a nanofiber structure that differs from the nanofiber structure of the outer nanofiber layer, said nanofiber core substrate further comprising an internal space surrounded by the inner nanofiber layer of the substrate, with one or more therapeutic agents positioned within said internal space; and a biocompatible polymeric coating surrounding said nanofiber core substrate.

Embodiment 15 is the implantable therapeutic delivery system of Embodiment 14, wherein the nanofiber core substrate comprises one or more middle nanofiber layers positioned between the inner and outer nanofiber layers of the substrate, each middle nanofiber layer comprising a nanofiber structure that differs from the nanofiber structure of the inner and outer nanofiber layers.

Embodiment 16 is the implantable therapeutic delivery system of Embodiment 14, wherein the nanofiber substrate is a cylindrical tube.

Embodiment 17 is the implantable therapeutic delivery system of Embodiment 16, wherein the cylindrical tube having a diameter of 0.1 mm to 30 cm.

Embodiment 18 is the implantable therapeutic delivery system of Embodiment 14, wherein the nanofiber substrate is a conical tube.

Embodiment 19 is the implantable therapeutic delivery system of any one of Embodiments 14-18, wherein nanofibers of the inner nanofiber layer and outer nanofiber layer independently have a diameter of 1 nm to 50 μm.

Embodiment 20 is the implantable therapeutic delivery system of any one of Embodiments 14-19, wherein the inner nanofiber layer and the outer nanofiber layer independently have a nanofiber density of 0.01 g/cm³ to 1.5 g/cm³.

Embodiment 21 is the implantable therapeutic delivery system of any one of Embodiments 14-20, wherein the inner nanofiber layer and the outer nanofiber layer independently have an average thickness of 1 μm to 5 mm.

Embodiment 22 is the implantable therapeutic delivery system of any one of Embodiments 14-21, wherein the inner nanofiber layer comprises pores, said pores having a diameter of 1 nm to 50 μm.

Embodiment 23 is the implantable therapeutic delivery system of any one of Embodiments 14-22, wherein the outer nanofiber layer comprises pores, said pores having a diameter of 1 nm to 50 μm.

Embodiment 24 is the implantable therapeutic delivery system of Embodiment 19, wherein the nanofiber structure of the inner nanofiber layer comprises a nanofiber density of <0.26 g/cm³ and the outer nanofiber layer comprises a nanofiber density of >0.26 g/cm³.

Embodiment 25 is the implantable therapeutic delivery system of Embodiment 19, wherein the nanofiber structure of the inner nanofiber layer comprises a nanofiber density of >0.26 g/cm³ and the outer nanofiber layer comprises a nanofiber density of <0.26 g/cm³.

Embodiment 26 is the implantable therapeutic delivery system of any one of Embodiments 14-23, wherein the inner and outer nanofiber layers comprise pores, and said pores of the inner nanofiber layer have a greater diameter than the pores of the outer nanofiber layer.

Embodiment 27 is the implantable therapeutic delivery system of any one of Embodiments 14-23, wherein the inner and outer nanofiber layers comprise pores, and said pores of the outer nanofiber layer have a greater diameter than the pores of the inner nanofiber layer

Embodiment 28 is the implantable therapeutic delivery system of any one of Embodiments 14-27, wherein the inner and outer nanofiber layers of the core substrate have a combined thickness of 1 μm to 5 mm.

Embodiment 29 is the implantable therapeutic delivery system of any one of Embodiments 1-28, wherein the nanofiber core substrate has a length of 0.5 cm to 1000 m.

Embodiment 30 is the implantable therapeutic delivery system of Embodiment 29, wherein the nanofiber core substrate has a length of 1 cm to 1 m.

Embodiment 31 is the implantable therapeutic delivery system of any of Embodiments 1-30, wherein the nanofiber core substrate comprises one or more biologically active agents selected from the group consisting of a protein, peptide, antibody or antibody fragment thereof, antibody mimetic, a nucleic acid, a small molecule, a hormone, a growth factor, an angiogenic factor, a cytokine, an anti-inflammatory agent, and combinations thereof.

Embodiment 32 is the implantable therapeutic delivery system of Embodiment 31, wherein the anti-inflammatory agent is selected from the group consisting of diclofenac, diflunisal, etodolac, fenoprofen, flurbiprofen, ibuprofen, indomethacin, ketoprofen, ketorolac, mefenamic acid, meloxicam, nabumetone, naproxen, oxaprozin, piroxicam, salsalate, sulindac, and tolmetin.

Embodiment 33 is the implantable therapeutic delivery system according to any of Embodiments 1-32, wherein the nanofiber core substrate comprises a material that is insoluble in the one or more biocompatible polymeric coatings surrounding the substrate.

Embodiment 34 is the implantable therapeutic delivery system according any one of Embodiments 1-33, wherein the nanofiber core substrate comprises a material selected from the group consisting of nylon, polyurethane, polysulfone, polyacrylonitrile, polyester such as polyethylene terephthalate and polybutester, polyvinylidene difluoride, polyacrylamide, poly (ethyl methacrylate), poly(methyl methacrylate), polyvinyl chloride, polyoxymethylene, polycarbonate, polypropylene, polyethylene, polybenzimidazole, polyaniline, polystyrene, polyvinylcarbazole, polyamide, poly vinyl phenol, cellulose acetate, polyacrylamide, poly(2-hydroxyethyl methacrylate), polyether imide, poly(ferrocenyldimethylsilane), poly(ethylene-co-vinylacetate), polyethylene-co-vinyl acetate, polyacrylic acid-polypyrene methanol, poly(ethylene-co-vinyl alcohol), polymetha-phenylene isophthalamide, poly(lactic acid), poly(ε-caprolactone), poly(lactic-co-glycolic acid), poly(l-lactide-co-ε-caprolactone), and combinations thereof.

Embodiment 35 is the implantable therapeutic delivery system of any one of Embodiments 1-34, wherein the nanofiber core substrate is translucent.

Embodiment 36 is the implantable therapeutic delivery system of claim 35, wherein the translucent nanofiber core substrate has >50% transmittance of light wave lengths between 400 nm and 800 nm.

Embodiment 37 is the implantable therapeutic delivery system of any one of Embodiments 1-36, wherein an elongated polymeric scaffold is positioned within the internal space of the nanofiber core substrate.

Embodiment 38 is the implantable therapeutic delivery system of Embodiment 37, wherein the elongated polymeric scaffold comprises a rod, tube, or film.

Embodiment 39 is the implantable therapeutic delivery system of Embodiment 37 or claim 38, wherein the elongated polymeric scaffold comprises a material selected from the group consisting of silicone, PDMS, rubber, nylon, polyurethane, polysulfone, polyacrylonitrile, polyester such as polyethylene terephthalate and polybutester, polyvinylidene difluoride, polyacrylamide, poly (ethyl methacrylate), poly(methyl methacrylate), polyvinyl chloride, polyoxymethylene, polycarbonate, polypropylene, polyethylene, polybenzimidazole, polyaniline, polystyrene, polyvinylcarbazole, polyamide, poly vinyl phenol, cellulose acetate, polyacrylamide, poly(2-hydroxyethyl methacrylate), polyether imide, poly(ferrocenyldimethylsilane), poly(ethylene-co-vinylacetate), polyethylene-co-vinyl acetate, polyacrylic acid-polypyrene methanol, poly(ethylene-co-vinyl alcohol), polymetha-phenylene isophthalamide, poly(lactic acid), poly(ε-caprolactone), poly(lactic-co-glycolic acid), poly(l-lactide-co-ε-caprolactone), and combinations thereof.

Embodiment 40 is the implantable therapeutic delivery system of any one of Embodiments 37-39, wherein the elongated polymeric scaffold comprises an internal fluidic space containing an oxygen carrier.

Embodiment 41 is the implantable therapeutic delivery system of Embodiment 40, wherein the oxygen carrier comprises a perfluorinated compound.

Embodiment 42 is the implantable therapeutic delivery system of Embodiment 41, wherein the perfluorinated compound is selected from the group consisting of perfluorotributylamine (FC-43), perfluorodecalin, perfluorooctyl bromide, bis-perfluorobutyl-ethene, perfluoro-4-methylmorpholine, perfluorotriethylamine, perfluoro-2-ethyltetrahydrofuran, perfluoro-2-butyltetrahydrofuran, perfluoropentane, perfluoro-2-methylpentane, perfluorohexane, perfluoro-4-isopropylmorpholine, perfluorodibutyl ether, perfluoroheptane, perfluorooctane, and mixtures thereof.

Embodiment 43 is the implantable therapeutic delivery system of any one of Embodiments 35-40, wherein the elongated polymeric scaffold comprises one or more therapeutic agents selected from the group consisting of therapeutic proteins, peptides, antibodies or fragments thereof, antibody mimetics, and other binding molecules, nucleic acids, small molecules, hormones, growth factors, angiogenic factors, cytokines, anti-inflammatory agents, and combinations thereof.

Embodiment 44 is the implantable therapeutic delivery system of Embodiment 43, wherein the anti-inflammatory agent is selected from the group consisting of diclofenac, diflunisal, etodolac, fenoprofen, flurbiprofen, ibuprofen, indomethacin, ketoprofen, ketorolac, mefenamic acid, meloxicam, nabumetone, naproxen, oxaprozin, piroxicam, salsalate, sulindac, and tolmetin.

Embodiment 45 is the implantable therapeutic delivery system of any one of Embodiments 1-44, wherein said internal space of the nanofiber core substrate is compartmentalized into two or more sub-internal spaces by one or more internal nanofiber walls.

Embodiment 46 is the implantable therapeutic delivery system of any one of Embodiments 1-45, wherein the one or more therapeutic agents positioned within the internal space of the nanofiber core substrate is selected from the group consisting of a protein, peptide, antibody or antibody fragment thereof, antibody mimetic, a nucleic acid, a small molecule, a hormone, a growth factor, an angiogenic factor, a cytokine, an anti-inflammatory agent, and combinations thereof.

Embodiment 47 is the implantable therapeutic delivery system of Embodiment 46, wherein the anti-inflammatory agent is selected from the group consisting of diclofenac, diflunisal, etodolac, fenoprofen, flurbiprofen, ibuprofen, indomethacin, ketoprofen, ketorolac, mefenamic acid, meloxicam, nabumetone, naproxen, oxaprozin, piroxicam, salsalate, sulindac, and tolmetin.

Embodiment 48 is the implantable therapeutic delivery system according to any one of Embodiments 1-45, wherein a preparation of cells is positioned in the internal space of the nanofiber core substrate and the one or more therapeutic agents is released from said preparation of cells.

Embodiment 49 is the implantable therapeutic delivery system according to Embodiment 48, wherein one or more hydrogel films, hydrogel capsules, hydrogel fibers, or hydrogel tubes embedded with the preparation of cells is positioned in the internal space of the nanofiber core substrate.

Embodiment 50 is the implantable therapeutic delivery system according to Embodiment 48, wherein a porous scaffold coated with hydrogel comprising the preparation of cells is positioned within the internal space of the nanofiber core substrate.

Embodiment 51 is the implantable therapeutic delivery system of Embodiment 50, wherein the porous scaffold comprises a material selected from the group consisting of silicone, PDMS, rubber, nylon, polyurethane, polysulfone, polyacrylonitrile, polyester such as polyethylene terephthalate and polybutester, polyvinylidene difluoride, polyacrylamide, poly (ethyl methacrylate), poly(methyl methacrylate), polyvinyl chloride, polyoxymethylene, polycarbonate, polypropylene, polyethylene, polybenzimidazole, polyaniline, polystyrene, polyvinylcarbazole, polyamide, poly vinyl phenol, cellulose acetate, polyacrylamide, poly(2-hydroxyethyl methacrylate), polyether imide, poly(ferrocenyldimethylsilane), poly(ethylene-co-vinylacetate), polyethylene-co-vinyl acetate, polyacrylic acid-polypyrene methanol, poly(ethylene-co-vinyl alcohol), polymetha-phenylene isophthalamide, poly(lactic acid), poly(ε-caprolactone), poly(lactic-co-glycolic acid), poly(l-lactide-co-ε-caprolactone), and combinations thereof.

Embodiment 52 is the implantable therapeutic delivery system of Embodiment 50, wherein the porous scaffold has pores having a diameter of between 1 nm and 500 μm.

Embodiment 53 is the implantable therapeutic delivery system of Embodiment 50, wherein the porous scaffold is a porous tube.

Embodiment 54 is the implantable therapeutic delivery system of Embodiment 53, wherein the porous tube comprises an internal fluidic space containing an oxygen carrier.

Embodiment 55 is the implantable therapeutic delivery system of Embodiment 54, wherein the oxygen carrier comprises a perfluorinated compound.

Embodiment 56 is the implantable therapeutic delivery system of Embodiment 55, wherein the perfluorinated compound is selected from the group consisting of perfluorotributylamine (FC-43), perfluorodecalin, perfluorooctyl bromide, bis-perfluorobutyl-ethene, perfluoro-4-methylmorpholine, perfluorotriethylamine, perfluoro-2-ethyltetrahydrofuran, perfluoro-2-butyltetrahydrofuran, perfluoropentane, perfluoro-2-methylpentane, perfluorohexane, perfluoro-4-isopropylmorpholine, perfluorodibutyl ether, perfluoroheptane, perfluorooctane, and mixtures thereof.

Embodiment 57 is the implantable therapeutic delivery system of any one of Embodiments 50-56, wherein the porous scaffold comprises one or more therapeutic agents selected from the group consisting of therapeutic proteins, peptides, antibodies or fragments thereof, antibody mimetics, and other binding molecules, nucleic acids, small molecules, hormones, growth factors, angiogenic factors, cytokines, anti-inflammatory agents, and combinations thereof.

Embodiment 58 is the implantable therapeutic delivery system of Embodiment 57, wherein the anti-inflammatory agent is selected from the group consisting of diclofenac, diflunisal, etodolac, fenoprofen, flurbiprofen, ibuprofen, indomethacin, ketoprofen, ketorolac, mefenamic acid, meloxicam, nabumetone, naproxen, oxaprozin, piroxicam, salsalate, sulindac, and tolmetin.

Embodiment 59 is the implantable therapeutic delivery system of Embodiment 48, wherein a cell growth matrix material embedded with the preparation of cells is positioned in the internal space of the nanofiber core substrate.

Embodiment 60 is the implantable therapeutic delivery system of Embodiment 59, wherein the cell growth matrix material is a hydrogel material.

Embodiment 61 is the implantable therapeutic delivery system of Embodiment 59, wherein the cell growth matrix material compromises a synthetic polymer selected from the group consisting of polyethylene glycol (PEG), poly(acrylic acid), poly(ethylene oxide), poly(vinyl alcohol), polyphosphazene, poly(hydroxyethyl methacrylate), triazole-zwitterion hydrogels, poly(sulfobetaine methacrylate), carboxybetaine methacrylate, poly[2-methacryloyloxyethyl phosphorylcholine, N-Hydroxyethyl acrylamide, copolymers thereof, derivatives thereof, and combinations thereof.

Embodiment 62 is the implantable therapeutic delivery system of Embodiment 59, wherein the cell growth matrix material compromises a natural polymeric material selected from the group consisting of collagen, elastin, fibrin, gelatin, gelatin-methacryloyl, silk fibroin, glycosaminoglycans, dextran, alginate, agarose, chitosan, bacterial cellulose, keratin, matrigel, decellularized hydrogels, and derivatives or combinations thereof.

Embodiment 63 is the implantable therapeutic delivery system of Embodiment 59, wherein the cell growth matrix material further comprises one or more cell factors to enhance cell growth, differentiation, and/or survival selected from the group consisting of glutamine, non-essential amino acids, epidermal growth factors, fibroblast growth factors, transforming growth factor/bone morphogenetic proteins, platelet derived growth factors, insulin growth factors, cytokines, fibronectin, laminin, heparin, collagen, glycosaminoglycan, proteoglycan, elastin, chitin derivatives, fibrin, and fibrinogen, FGF, bFGF, acid FGF (aFGF), FGF-2, FGF-4, EGF, PDGF, TGF-beta, angiopoietin-1, angiopoietin-2, placental growth factor (PlGF), VEGF, PMA (phorbol 12-myristate 13-acetate), combinations thereof.

Embodiment 64 is the implantable therapeutic delivery system of any one of Embodiments 48-63, wherein the preparation of cells is a preparation of single cells or a preparation of cell aggregates.

Embodiment 65 is the implantable therapeutic delivery system of any one of Embodiments 48-64, wherein the preparation of cells is a preparation of primary cells or a preparation of immortalized cells.

Embodiment 66 is the implantable therapeutic delivery system of any one of Embodiments 48-65, wherein the preparation of cells is a preparation of mammalian cells.

Embodiment 67 is the implantable therapeutic delivery system of any one of Embodiments 48-66, wherein the preparation of cells is selected from the group consisting of a preparation of primate cells, rodent cells, canine cells, feline cells, equine cells, bovine cells, and porcine cells.

Embodiment 68 is the implantable therapeutic delivery system of any one of Embodiments 48-67, wherein the preparation of cells is a preparation of human cells.

Embodiment 69 is the implantable therapeutic delivery system of any one of Embodiments 48-68, wherein the preparation of cells is a preparation of stem cells or stem cell derived cells.

Embodiment 70 is the implantable therapeutic delivery system of Embodiment 69, wherein the stem cells are pluripotent, multipotent, oligopotent, or unipotent stem cells.

Embodiment 71 is the implantable therapeutic delivery system of Embodiment 69, wherein the preparation of stem cells is selected from the group consisting of embryonic stem cells, epiblast cells, primitive ectoderm cells, primordial germ cells, and induced pluripotent stem cells.

Embodiment 72 is the implantable therapeutic delivery system of any one of Embodiments 48-68, wherein the preparation of cells is a preparation of cells selected from the group consisting of smooth muscle cells, cardiac myocytes, platelets, epithelial cells, endothelial cells, urothelial cells, fibroblasts, embryonic fibroblasts, myoblasts, chondrocytes, chondroblasts, osteoblasts, osteoclasts, keratinocytes, hepatocytes, bile duct cells, islet cells, thyroid, parathyroid, adrenal, hypothalamic, pituitary, ovarian, testicular, salivary gland cells, adipocytes, embryonic stem cells, mesenchymal stem cells, neural cells, endothelial progenitor cells, hematopoietic cells, precursor cells, mesenchymal stromal cells, Baby Hamster Kidney (BHK) cells, Chinese Hamster Ovary cells, Human Amniotic Epithelial (HAE) cells, choroid plexus cells, chromaffin cells, adrenal chromaffin cells, pheochomocytoma cell line PC12, human retinal pigment epithelium cells, recombinant human retinal pigment epithelium cells, NGF-secreting Baby Hamster Kidney (BHK) cells, human bone marrow-derived stem cells transfected with GLP-1, BDNF-producing fibroblasts, NGF-producing cells, CNTF-producing cells, BDNF-secreting Schwann cells, IL-2-secreting myoblasts, endostatin-secreting cells, and cytochrome P450 enzyme overexpressed feline kidney epithelial cells, myogenic cells, embryonic stem cell-derived neural progenitor cells, irradiated tumor cells, proximal tubule cells, neural precursor cells, astrocytes, genetically engineered cells.

Embodiment 73 is the implantable therapeutic delivery system of Embodiment 66, wherein the preparation of cells is a preparation comprising islet cells that release insulin and glucagon.

Embodiment 74 is the implantable therapeutic delivery system of Embodiment 67, wherein the preparation comprising islet cells is a preparation of human cells, porcine cells, or rodent cells.

Embodiment 75 is the implantable therapeutic delivery system of Embodiments 67 or 68, wherein the preparation of cells comprises an islet density between 1×10³ to 6×10⁵ islet equivalents (IEQs)/mL.

Embodiment 76 is the implantable therapeutic delivery system according to any one of Embodiments 48-74, wherein the preparation of cell comprises a cell density between 1×10³ to 6×10¹⁰ cells/mL.

Embodiment 77 is the implantable therapeutic delivery system of any one of Embodiments 1-76, wherein said proximal and distal ends of the nanofiber core substrate are sealed.

Embodiment 78 is the implantable therapeutic delivery system of Embodiment 77, wherein the proximal and distal ends of the nanofiber core substrate are sealed by a heat seal, a suture knot, a clamp, a rubber seal, or a screw closure.

Embodiment 79 is the implantable therapeutic delivery system of any one of Embodiments 2-78, wherein the outer biocompatible polymeric coating is a hydrogel material.

Embodiment 80 is the implantable therapeutic delivery system of Embodiments 79, wherein the hydrogel material is a synthetic polymer selected from the group consisting of polyethylene glycol (PEG), poly(acrylic acid), poly(ethylene oxide), poly(vinyl alcohol), polyphosphazene, poly(hydroxyethyl methacrylate), triazole-zwitterion hydrogels (TR-qCB, TR-CB, TR-SB), poly(sulfobetaine methacrylate), carboxybetaine methacrylate, poly[2-methacryloyloxyethyl phosphorylcholine, N-hydroxyethyl acrylamide, a copolymer thereof, a derivatives thereof, and a combination thereof.

Embodiment 81 is the implantable therapeutic delivery system of Embodiment 79, wherein the hydrogel material is a natural polymeric material selected from the group consisting of collagen, elastin, fibrin, gelatin, gelatin-methacryloyl, silk fibroin, glycosaminoglycans, dextran, alginate, agarose, chitosan, bacterial cellulose, keratin, matrigel, decellularized hydrogels, derivatives thereof, and combinations thereof.

Embodiment 82 is the implantable therapeutic delivery system of Embodiment 79, wherein the hydrogel material is a zwitterionically modified hydrogel.

Embodiment 83 is the implantable therapeutic delivery system of Embodiment 79, wherein the hydrogel material comprises a pure alginate, a modified alginate, or a mixture of pure and modified alginate.

Embodiment 84 is the implantable therapeutic delivery system of Embodiment 83, wherein the modified alginate is a zwitterionically modified alginate.

Embodiment 85 is the implantable therapeutic delivery system of Embodiment 79, wherein the hydrogel material comprises a mixture of pure alginate and modified alginate in a ratio of about 1:1000 to 1000:1 (v/v).

Embodiment 86 is the implantable therapeutic delivery system of Embodiment 79, wherein the hydrogel material comprises a mixture of pure alginate and modified alginate in a ratio of about 3:7 to 7:3 (v/v).

Embodiment 87 is the implantable therapeutic delivery system of any one of Embodiments 2-86, wherein the biocompatible polymeric coating is crosslinked and interlocked to the nanofiber core substrate.

Embodiment 88 is the implantable therapeutic delivery system of any one of Embodiments 2-86, wherein the biocompatible polymeric coating comprises one or more biologically active agents selected from the group consisting of a protein, peptide, antibody or antibody fragment thereof, antibody mimetic, a nucleic acid, a small molecule, a hormone, a growth factor, an angiogenic factor, a cytokine, an anti-inflammatory agent, and combinations thereof.

Embodiment 89 is the implantable therapeutic delivery system of Embodiment 88, wherein the biocompatible polymer coating comprises an anti-inflammatory agent selected from the group consisting of diclofenac, diflunisal, etodolac, fenoprofen, flurbiprofen, ibuprofen, indomethacin, ketoprofen, ketorolac, mefenamic acid, meloxicam, nabumetone, naproxen, oxaprozin, piroxicam, salsalate, sulindac, and tolmetin.

Embodiment 90 is the implantable therapeutic delivery system of Embodiment 1, wherein the hydrogel surrounding the nanofiber core substrate is crosslinked and interlocked to the nanofiber core substrate.

Embodiment 91 is the implantable therapeutic delivery system of Embodiment 1, wherein the hydrogel surrounding the nanofiber core substrate has a thickness of 1 nm to 5 mm, wherein the standard deviation in thickness of the hydrogel around the entirety of the nanofiber core substrate is <100%.

Embodiment 92 is the implantable therapeutic delivery system of Embodiment 1, wherein the hydrogel surrounding the nanofiber core substrate comprises one or more biologically active agents selected from the group consisting of a protein, peptide, antibody or antibody fragment thereof, antibody mimetic, a nucleic acid, a small molecule, a hormone, a growth factor, an angiogenic factor, a cytokine, an anti-inflammatory agent, and combinations thereof.

Embodiment 93 is the implantable therapeutic delivery system of Embodiment 92, wherein the hydrogel surrounding the nanofiber core substrate comprises an anti-inflammatory agent selected from the group consisting of diclofenac, diflunisal, etodolac, fenoprofen, flurbiprofen, ibuprofen, indomethacin, ketoprofen, ketorolac, mefenamic acid, meloxicam, nabumetone, naproxen, oxaprozin, piroxicam, salsalate, sulindac, and tolmetin

Embodiment 94 is the implantable therapeutic delivery system of Embodiment 14, wherein the biocompatible polymeric coating surrounding the nanofiber core substrate has a thickness of 1 nm to 5 mm, wherein the standard deviation in thickness of the polymeric coating around the entirety of the nanofiber core substrate is <100%.

Embodiment 95 is a method of delivering a therapeutic agent to a subject in need thereof, said method comprising: implanting the implantable therapeutic delivery system according to any one of Embodiments 1-94 into the subject.

Embodiment 96 is the method of treating diabetes in a subject, said method comprising: implanting the implantable therapeutic delivery system according to any one of Embodiments 1-94 into the subject having diabetes.

Embodiment 97 is the method of Embodiment 96, wherein the one or more therapeutic agents of the implantable therapeutic delivery system is insulin, glucagon, or a combination thereof released from a preparation of cells positioned in the internal space of the nanofiber core substrate.

Embodiment 98 is the method of Embodiment 97, wherein the preparation of cells comprises a preparation of islets.

Embodiment 99 is the method of Embodiment 98, wherein the preparation of islets is a preparation of primate islets, rodent islets, canine islets, feline islets, equine islets, bovine islets, or porcine islets.

Embodiment 100 is the method of Embodiment 98, wherein the preparation of islets is derived from a preparation of stem cells.

Embodiment 101 is the method of Embodiment 100, wherein the preparation of stem cells is a preparation of pluripotent, multipotent, oligopotent, or unipotent stem cells.

Embodiment 102 is the method of Embodiment 100, wherein the preparation of stem cells is selected from the group consisting of embryonic stem cells, epiblast cells, primitive ectoderm cells, primordial germ cells, and induced pluripotent stem cells.

Embodiment 103 is a method of treating a bleeding disorder in a subject, said method comprising: implanting the implantable therapeutic delivery system of any one of Embodiments 1-94 into the subject having a bleeding disorder.

Embodiment 104 is the method of Embodiment 103, wherein the bleeding disorder is selected from the group consisting of hemophilia A, hemophilia B, von Willebrand disease, Factor I deficiency, Factor II deficiency, Factor V deficiency, Factor VII deficiency, Factor X deficiency, Factor XI deficiency, Factor XII deficiency, and Factor XIII deficiency.

Embodiment 105 is the method of Embodiment 103, wherein the one or more therapeutic agents is a blood clotting factor released from a preparation of cells positioned in the internal space of the nanofiber core substrate.

Embodiment 106 is the method of Embodiment 105, wherein the preparation of cells comprises recombinant myoblasts, mesenchymal stromal cells, induced pluripotent stem cell derived endothelial cells, or a combination thereof.

Embodiment 107 is the method of Embodiment 105, wherein the blood clotting factor is selected from the group consisting of Factor I, Factor II, Factor V, Factor VII, Factor VIII, Factor IX, Factor X, Factor XI, Factor XII, Factor XIII, and combinations thereof.

Embodiment 108 is the method of treating a lysosomal storage disease in a subject, said method comprising: implanting the implantable therapeutic delivery system of any one of Embodiments 1-94 into the subject having the lysosomal storage disease.

Embodiment 109 is the method of Embodiment 108, wherein the one or more therapeutic agents is an enzyme released from a preparation of cells positioned in the internal space of the nanofiber core substrate.

Embodiment 110 is the method of Embodiment 109, wherein the preparation of cells comprises hematopoietic stem cells, fibroblasts, myoblasts, Baby Hamster Kidney (BHK) cells, Chinese Hamster Ovary cells, Human Amniotic Epithelial (HAE) cells, or combinations thereof.

Embodiment 111 is the method of Embodiment 109, wherein the enzyme is selected from the group consisting of α-L-iduronidase, Iduronate-2-sulfatase, α-glucuronidase, Arylsulfatase A, alpha-Galactosidase A, and combinations thereof.

Embodiment 112 is the method of treating a neurological disorder in a subject, said method comprising: implanting the implantable therapeutic delivery system of any one of Embodiments 1-94 into the subject having the neurological disorder.

Embodiment 113 is the method of Embodiment 112, wherein the neurological disorder is a sensory disorder.

Embodiment 114 is the method of Embodiment 113, wherein the neurological disorder is selected from the group consisting of Parkinson's disorder, Alzheimer's disease, epilepsy, Huntington's disease, Amyotrophic lateral sclerosis, chronic pain, visual loss, hearing loss, peripheral nerve injury, and spinal cord injury.

Embodiment 115 is the method of Embodiment 112, wherein the one or more therapeutic agents is a therapeutic molecule released from a preparation of cells positioned in the internal space of the nanofiber core substrate.

Embodiment 116 is the method of Embodiment 115, wherein the preparation of cells comprises choroid plexus cells, chromaffin cells, pheochomocytoma cell line PC12, human retinal pigment epithelial cells, NGF-secreting Baby Hamster Kidney (BHK) cells, myoblasts, human bone marrow-derived stem cells transfected with GLP-1, BDNF-producing fibroblasts, NGF-producing cells, CNTF-producing cells, adrenal chromaffin cells, BDNF-secreting Schwann cells, myogenic cells, embryonic stem cell-derived neural progenitor cells, and combinations thereof.

Embodiment 117 is the method of Embodiment 115, wherein the therapeutic molecule is selected from the group consisting of cerebrospinal fluid, extracellular fluid, levodopa, nerve growth factor (NGF), ciliary neurotrophic factor (CNTF), BLP-1, brain-derived neurotrophic factor (BDNF), vascular endothelial growth factor (VEGF), enkephalin, adrenaline, catecholamine, and combinations thereof.

Embodiment 118 is the method of treating a cancer in a subject, said method comprising: implanting the implantable therapeutic delivery system of any one of Embodiments 1-94 into the subject having cancer.

Embodiment 119 is the method of Embodiment 118, wherein the one or more therapeutic agents is a therapeutic molecule released from a preparation of cells positioned in the internal space of the nanofiber core substrate.

Embodiment 120 is the method of Embodiment 119, wherein the preparation of cells comprises IL-2-secreting myoblasts, endostatin-secreting cells, Chinese Hamster Ovary cells, and cytochrome P450 enzyme overexpressed feline kidney epithelial cells, irradiated tumor cells, and combinations thereof.

Embodiment 121 is the method of Embodiment 120, wherein the therapeutic molecule is selected from IL-2, endostatin, cytochrome P450 enzyme, tumor antigens, a cytokine, and combinations thereof.

Embodiment 122 is the method of treating a chronic eye disease in a subject, said method comprising: implanting the implantable therapeutic delivery system of any one of Embodiments 1-94 into the subject having a chronic eye disease.

Embodiment 123 is the method of Embodiment 122 further comprising: administering one or more trophic factors to the subject to protect compromised retinal neurons and to restore neural circuits.

Embodiment 124 is the method of Embodiment 122, wherein the chronic eye disease is selected from the group consisting of age-related macular degeneration, diabetic retinopathy, retinitis pigmentosa, glaucoma, macular telangiectasia, and combinations thereof.

Embodiment 125 is the method of Embodiment 122, wherein the one or more therapeutic agents is a therapeutic molecule released from a preparation of cells positioned in the internal space of the nanofiber core substrate.

Embodiment 126 is the method of Embodiment 125, wherein the preparation of cells comprises human retinal pigment epithelium cells, recombinant human retinal pigment epithelium cells, or a combination thereof.

Embodiment 127 is the method of Embodiment 125, wherein the therapeutic molecule is selected from the group consisting of ciliary neurotrophic factor, antagonists against vascular endothelial growth factor and platelet-derived growth factor, and combinations thereof.

Embodiment 128 is the method of treating a kidney failure in a subject, said method comprising: implanting the implantable therapeutic delivery system of any one of Embodiments 1-94 into the subject having a kidney failure.

Embodiment 129 is the method of Embodiment 128, wherein the one or more therapeutic agents is a therapeutic molecule released from a preparation of cells positioned in the internal space of the nanofiber core substrate.

Embodiment 130 is the method of Embodiment 129, wherein the preparation of cells comprises renal proximal tubule cells, mesenchymal stem cells, and a combination thereof.

Embodiment 131 is the method of treating a chronic pain in a subject, said method comprising: implanting the implantable therapeutic delivery system of any one of Embodiments 1-94 into the subject having a chronic pain.

Embodiment 132 is the method of Embodiment 131, wherein the chronic pain is chronic pain caused by degenerative back and knee, neuropathic back and knee, or cancer.

Embodiment 133 is the method of Embodiment 131, wherein the one or more therapeutic agents is a therapeutic molecule released from a preparation of cells positioned in the internal space of the nanofiber core substrate.

Embodiment 134 is the method of Embodiment 133, wherein the preparation of cells comprises chromaffin cells, neural precursor cells, mesenchymal stem cells, astrocytes, and genetically engineered cells, or a combination thereof.

Embodiment 135 is the method of Embodiment 133, wherein the therapeutic molecule is selected from the group consisting of catecholamine, opioid peptides, enkephalins, and combinations thereof.

Embodiment 136 is the method according to any one of Embodiments 95-135, wherein said implanting is carried out via a laparoscopic procedure.

Embodiment 137 is the method according to any one of Embodiments 95-135, wherein said therapeutic delivery system is implanted intraperitoneally, percutaneously, or subcutaneously.

Embodiment 138 is the method according to any one of Embodiments 95-135, wherein said implanting involves suturing the delivery system to a body wall of the subject.

Embodiment 139 is the method according to any one of Embodiments 95-135, wherein said implanting involves anchoring the delivery system to a body wall of the subject via a transabdominal portal.

Embodiment 140 is the method according to any one of Embodiments 95-135, wherein said implanting involves wrapping the delivery system in omentum of the subject.

Embodiment 141 is the method according to any one of Embodiments 95-135, wherein said implanting involves positioning the delivery system in a cavity between the liver and the diaphragm.

Embodiment 142 is the method according to any one of Embodiments 95-135, wherein said implanting involves anchoring the delivery system to the diaphragm.

Embodiment 143 is the method according to any one of Embodiments 95-142, wherein said method further comprises: retrieving the implantable therapeutic delivery system from the subject.

Embodiment 144 is the method according to Embodiment 143, wherein said method further comprises: implanting a replacement implantable therapeutic delivery system after said retrieving.

Embodiment 145 is the method of making a nanofiber core substrate according to any of Embodiments 1-94, said method comprising: providing one or more polymer solutions comprising 1% to 50% polymer in a solvent; electrospinning said one or more polymer solutions onto a rotating collecting rod, wherein said collecting rod is coated with a viscous saccharide solution, to form the nanofiber core substrate; and removing the porous nanofiber core substrate from the collecting rod.

Embodiment 146 is the method of Embodiment 145, wherein said one or more polymer solutions are electrospun using a single channel nozzle or a multi-channel nozzle comprising needles of different diameters.

Embodiment 147 is the method of Embodiment 145, wherein said removing comprises: dissolving the saccharide solution from the collecting rod in water.

Embodiment 148 is the method of Embodiment 145, wherein the solvent comprising hexafluoroisopropanol (HFIP)

Embodiment 149 is the method of Embodiment 145, wherein said solvent is pure HFIP.

Embodiment 150 is the method of Embodiment 145, wherein said solvent comprises a mixture of HFIP and formic acid.

Embodiment 151 is the method of Embodiment 145, wherein the polymer solution comprises one or more polymers selected from the group consisting of nylon, polyurethane, polysulfone, polyacrylonitrile, polyester such as polyethylene terephthalate and polybutester, polyvinylidene difluoride, polyacrylamide, poly (ethyl methacrylate), poly(methyl methacrylate), polyvinyl chloride, polyoxymethylene, polycarbonate, polypropylene, polyethylene, polybenzimidazole, polyaniline, polystyrene, polyvinylcarbazole, polyamide, poly vinyl phenol, cellulose acetate, polyacrylamide, poly(2-hydroxyethyl methacrylate), polyether imide, poly(ferrocenyldimethylsilane), poly(ethylene-co-vinylacetate), polyethylene-co-vinyl acetate, polyacrylic acid-polypyrene methanol, poly(ethylene-co-vinyl alcohol), polymetha-phenylene isophthalamide, poly(lactic acid), poly(ε-caprolactone), poly(lactic-co-glycolic acid), and poly(l-lactide-co-ε-caprolactone).

Embodiment 152 is the method of Embodiment 145, wherein the viscous saccharide solution comprises one or more monosaccharides, disaccharides, oligosaccharides, and mixtures thereof.

Embodiment 153 is the method of Embodiment 145, wherein the saccharide solution comprises glucose, galactose, fructose, sucrose, lactose, maltose, trehalose, and mixtures thereof.

Embodiment 154 is the method of Embodiment 145, wherein the saccharide solution has a viscosity of >4×10³ mPa·s.

Embodiment 155 is the method of Embodiments 145, wherein the viscous saccharide solution is a sucrose solution comprising about 25 g/mL sucrose.

Embodiment 156 is the method according to any one of Embodiments 145-156, where in the method further comprises: applying a solvent vapor to the collecting rod during said electrospinning under conditions effective to generate a translucent porous nanofiber substrate.

Embodiment 157 is the method of producing an implantable therapeutic delivery system, said method comprising: providing a longitudinally extending nanofiber core substrate, said substrate having a proximal and a distal end, each proximal and distal end having an opening to at least one internal space within the nanofiber core substrate; sealing the proximal end of the nanofiber core substrate; soaking the sealed proximal end and outer surface of the nanofiber core substrate in a biocompatible polymer solution to allow polymer solution penetration into the nanofiber core substrate; filling the at least one internal space of the nanofiber core substrate with one or more crosslinking agents to crosslink the coated biocompatible polymer solution to the nanofiber core substrate; loading one or more therapeutic agents into the at least one internal space of the nanofiber core substrate through the opening at the distal end of the nanofiber core substrate; sealing the distal end of the loaded nanofiber core substrate; and coating the sealed distal end of the nanofiber core substrate with the biocompatible polymer solution to form the implantable therapeutic delivery system.

Embodiment 158 is the method of producing an implantable therapeutic delivery system, said method comprising: providing a longitudinally extending nanofiber core substrate, said substrate having a proximal and a distal end, each proximal and distal end having an opening to at least one internal space within the nanofiber core substrate; sealing the proximal end of the nanofiber core substrate; applying a biocompatible polymer solution to the sealed proximal end and outer surface of the nanofiber core substrate; loading one or more therapeutic agents into the at least one internal space of the nanofiber core substrate through the opening at the distal end of the nanofiber core substrate; sealing the distal end of the loaded nanofiber core substrate; applying the biocompatible polymer solution to the sealed distal end of the nanofiber core substrate; and crosslinking the coated biocompatible polymer solution to the nanofiber core substrate to form the implantable therapeutic delivery system.

Embodiment 159 is the method of producing an implantable therapeutic delivery system, said method comprising: providing a longitudinally extending nanofiber core substrate, said substrate having a proximal and a distal end, each proximal and distal end having an opening to at least one internal space within the nanofiber core substrate; sealing the proximal end of the nanofiber core substrate; loading one or more therapeutic agents into the at least one internal space of the nanofiber core substrate through the opening at the distal end of the nanofiber core substrate; sealing the distal end of the loaded nanofiber core substrate; soaking the sealed and loaded nanofiber core substrate in a cross-linker solution; coating the cross-linker soaked nanofiber core substrate with a biocompatible polymer solution; and crosslinking the coated biocompatible polymer solution to the nanofiber core substrate to form the implantable therapeutic delivery system.

Embodiment 160 is the method of any one of Embodiments 157-159, wherein said loading comprises: positioning one or more hydrogel films, hydrogel capsules, hydrogel fibers, hydrogel tubes, or a combination thereof within the at least one internal space, wherein said one or more films, capsules, fibers or tubes are embedded with a preparation of cells that release the one or more therapeutic agents.

Embodiment 161 is the method of any one of Embodiments 157-159, wherein said loading comprises: providing a porous scaffold coated with an outer layer of hydrogel, said hydrogel embedded with a preparation of cells that release the one or more therapeutic agents, and positioning the porous scaffold coated with hydrogel embedded cells within the at least one internal space of the nanofiber core substrate.

Embodiment 162 is the method of any one of Embodiments 157-159, wherein said loading comprises: providing a mixture of extracellular matrix precursor material and cells; loading said mixture into the at least one internal space through the distal end of the nanofiber core substrate, and crosslinking the extracellular matrix material.

Embodiment 1633 is the method of any one of Embodiments 157-159, wherein said sealing is carried out using a thermo sealer.

Embodiment 164 is the method of any one of Embodiments 157-159, wherein the one or more outer biocompatible polymeric solution comprises a hydrogel material.

Embodiment 165 is the method of Embodiment 164, wherein the hydrogel material is a synthetic polymer selected from the group consisting polyethylene glycol (PEG), poly(acrylic acid), poly(ethylene oxide), poly(vinyl alcohol), polyphosphazene, poly(hydroxyethyl methacrylate), triazole-zwitterion hydrogels, poly(sulfobetaine methacrylate), carboxybetaine methacrylate, poly[2-methacryloyloxyethyl phosphorylcholine, N-Hydroxyethyl acrylamide, copolymers thereof, derivatives thereof, and combinations thereof.

Embodiment 166 is the method of Embodiment 164, wherein the hydrogel material is a natural polymeric material selected from the group consisting of collagen, elastin, fibrin, gelatin, gelatin-methacryloyl, silk fibroin, glycosaminoglycans, dextran, alginate, agarose, chitosan, bacterial cellulose, keratin, matrigel, decellularized hydrogels, derivatives thereof, and combinations thereof.

Embodiment 167 is the method of Embodiments 164, wherein the hydrogel material is a zwitterionically modified hydrogel.

Embodiment 168 is the method of Embodiment 164, wherein the hydrogel material comprises a pure alginate, a modified alginate, or a mixture of pure and modified alginate.

Embodiment 169 is the method of Embodiment 168, wherein the modified alginate is a zwitterionically modified alginate.

Embodiment 170 is the method of Embodiment 168, wherein the hydrogel material comprises a mixture of pure alginate and modified alginate in a ratio of about 1:1000 to 1000:1 (v/v).

Embodiment 171 is the method of Embodiment 168, wherein the hydrogel material comprises a mixture of pure alginate and modified alginate in a ratio of about 3:7 to 7:3 (v/v).

Embodiment 172 is the method of Embodiments 158 or 159, wherein said crosslinking comprises: exposing the biocompatible polymer solution to one or more crosslinking agents.

Embodiment 173 is the method of Embodiment 172, wherein the one or more crosslinking agents is a cation selected from Ba²⁺, Ca²⁺, Cd²⁺, Cu²⁺, Fe²⁺, Mg²⁺, Mn²⁺, Ni²⁺, Pb²⁺, Sn²⁺, Sr²⁺, and Zn²⁺.

Embodiment 174 is the method of producing a porous nanofiber substrate, said method comprising: providing one or more polymer-solvent solutions; coating a rotating collecting rod with a viscous saccharide solution; electrospinning said one or more polymer solutions onto the coated rotating collecting rod to form the porous nanofiber substrate; and dissolving the viscous saccharide solution from the collecting rod, thereby removing the porous nanofiber substrate from the collecting rod.

Embodiment 175 is the method of Embodiment 174, wherein the viscous saccharide solution comprises one or more monosaccharides, disaccharides, oligosaccharides, and mixtures thereof.

Embodiment 176 is the method of Embodiments 174, wherein the viscous saccharide solution comprises glucose, galactose, fructose, sucrose, lactose, maltose, trehalose, and mixtures thereof.

Embodiment 177 is the method of Embodiment 174, wherein the saccharide solution has a viscosity of >4×10³ mPa·s.

Embodiment 178 is the method of Embodiment 174, wherein the viscous saccharide solution is a sucrose solution comprising about 25 g/mL sucrose.

Embodiment 179 is the method of Embodiment 174, wherein the solvent comprises hexafluoroisopropanol (HFIP).

Embodiment 180 is the method of Embodiment 174, wherein the polymer solution is a 1% to 50% polymer solution.

Embodiment 181 is the method of Embodiment 174, wherein the polymer solution comprises one or more polymers selected from the group consisting of nylon, polyurethane, polysulfone, polyacrylonitrile, polyester such as polyethylene terephthalate and polybutester, polyvinylidene difluoride, polyacrylamide, poly (ethyl methacrylate), poly(methyl methacrylate), polyvinyl chloride, polyoxymethylene, polycarbonate, polypropylene, polyethylene, polybenzimidazole, polyaniline, polystyrene, polyvinylcarbazole, polyamide, poly vinyl phenol, cellulose acetate, polyacrylamide, poly(2-hydroxyethyl methacrylate), polyether imide, poly(ferrocenyldimethylsilane), poly(ethylene-co-vinylacetate), polyethylene-co-vinyl acetate, polyacrylic acid-polypyrene methanol, poly(ethylene-co-vinyl alcohol), polymetha-phenylene isophthalamide, poly(lactic acid), poly(ε-caprolactone), poly(lactic-co-glycolic acid), and poly(l-lactide-co-ε-caprolactone)

Embodiment 182 is a thermo sealing device comprising: a first substrate portion comprising a cut-out along its peripheral edge; a second substrate portion comprising a cut-out that is substantially identical in shape and size to the cut-out of the first substrate, said second substrate further comprising a trench configured to house a heating element, wherein said trench aligns with the cut-out of the second substrate; a connector connecting the first and second substrate portions in a manner that aligns the cut-out of the first substrate portion with the cut-out of the second substrate portion; and a heating element positioned in the trench of the second substrate portion.

Embodiment 183 is the thermo sealing device of Embodiment 182, wherein the first and second substrate portions are made from a separate pieces of substrate material.

Embodiment 184 is the thermo sealing device of Embodiment 182, wherein the first and second substrate portions are made from a single piece of substrate material.

Embodiment 185 is the thermo sealing device of Embodiment 182, wherein the first and second substrate portions are comprised of a heat resistant material.

Embodiment 186 is the thermo sealing device of Embodiment 182, wherein the heat resistant material is a ceramic material or a metal material.

Embodiment 187 is the thermo sealing device of Embodiment 182, wherein the heat resistant material is a thermosetting material.

Embodiment 188 is the thermo sealing device of Embodiment 187, wherein the thermosetting material is selected from the group consisting of polydimethylsiloxane, epoxy resin, melamine formaldehyde, polyester resin, urea formaldehyde, and phenol formaldehyde.

Embodiment 189 is the thermo sealing device of Embodiment 187, wherein the thermosetting material is a transparent material.

Embodiment 190 is the thermo sealing device of Embodiment 182, wherein the cut-out of the first and second substrate portions has a rounded edge.

Embodiment 191 is the thermo sealing device of Embodiment 182, wherein the heating element is a ribbon-like structure.

Embodiment 192 is the thermo sealing device of Embodiment 182, wherein the heating element is flat and positioned in the trench standing on its narrow edge.

### EXAMPLES

### Materials and Methods

Materials

Poly (caprolactam) (nylon 6, 181110), formic acid (FA, F0507), thrombin from bovine plasma (T4648), fibrinogen from bovine plasma (F8630), streptozotocin (STZ, 50130) and gelatin from porcine skin (G1890) were purchased from Sigma-Aldrich Co (St. Louis, Mo.). 1,1,1,3,3,3-Hexafluoro-2-propanol (HFIP, 003409) was purchased from Oakwood Products, Inc. (Estill, S.C.). Calcium chloride (CaCl₂, BDH9224) and sodium chloride (NaCl, BDH9286) were purchased from VWR International (Radnor, Pa.). Barium chloride dihydrate (BaCl₂.2H₂O, BX0060-1) was purchased from EMD Millipore Corporation (Burlington, Mass.). Regular sodium alginate (PROTANAL LF 10/60FT) and sterile sodium alginate (Pronova SLG 100) were purchased from FMC BioPolymer Co (Philadelphia, Pa.). Sucrose (8360-6) was purchased from Avantor Performance Materials, LLC. (Center Valley, Pa.). All reagents were used without further purification. Sulfobetaine-modified alginate (SB-alginate) was synthesized according to a previously published protocol (Liu et al., “Zwitterionically Modified Alginates Mitigate Cellular Overgrowth for Cell Encapsulation,” Nat. Commun. 10:1-14 (2019), which is hereby incorporated by reference in its entirety).

Animals

C57BL/6J mice were purchased from Jackson Lab. SCID-beige mice were purchased from Taconic Farms. Sprague-Dawley rats were purchased from Charles River Laboratories. Beagle dogs were purchased from Marshall Bioresources. All animal procedures were approved by the Cornell Institutional Animal Care and Use Committee.

Statistical Analysis

Results were presented as average±standard deviation. Statistical analysis was conducted by GraphPad Prism 8.0.1. Unpaired t test was performed when two groups were compared, while one-way ANOVA with a Tukey's multiple comparisons test was performed when more than two groups were compared. Statistical significance was determined as n.s. or *, **, ***, ****, when p-value was <0.05, <0.01, <0.001, <0.0001, respectively.

### Example 1—Electrospinning

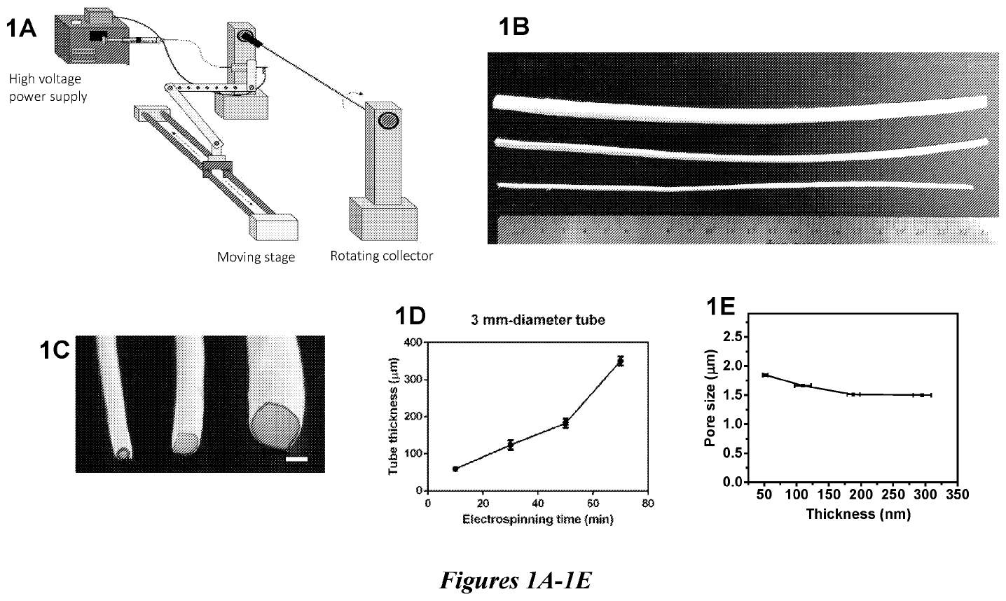

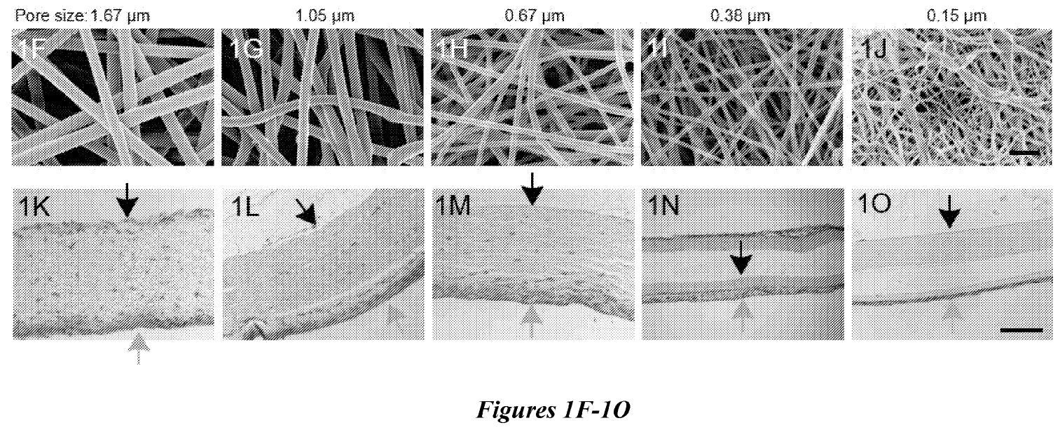

Nanofiber tubes were fabricated by electrospinning of nylon 6 (PA6) solutions. To fabricate nanofiber tubes with controllable fiber diameter, pore size, thickness, and good uniformity, a customized electrospinning setup was developed for the SHIELD device (FIG. 1A). In particular, the uniformity was achieved by using a rotating collector and a moving stage. Both speeds were precisely regulated by a controller. Specifically, the moving stage enables the back-and-forth movement of the spinning nozzle and thus the uniform deposition of nanofibers on the collector that rotates simultaneously. Importantly, both the travel length of the moving stage and the length of the collector can be adjusted with ease to fabricate tubes with different lengths. Unless otherwise noted, the diameter of rod collectors, collecting distance, rotating speed of collecting rod, and the speed of moving stage were kept constant at 3.2 mm, 8 cm, 375 rpm, 3.48 m min⁻¹, respectively. Detailed electrospinning parameters for different pore sizes can be found in Table 1. It should be noted nanofiber membranes with an average pore size of 1.05 μm were used for most studies unless otherwise noted.

TABLE 1
Parameters for electrospun nanofiber tubes with different average pore sizes.

| | Pumping speed | Voltage | Electrospinning time | Pore size |
|---|---|---|---|---|
| Solution | (mL h⁻¹) | (kV) | (min) | (μm) |
| 20% PA6/HFIP | 2.4 | 15 | 20 | 1.67 |
| 25% PA6/(HFIP/FA 8/2 v/v) | 1.2 | 18 | 32 | 1.05 |
| 20% PA6/(HFIP/FA 8/2 v/v) | 1.2 | 18 | 40 | 0.67 |
| 15% PA6/(HFIP/FA 8/2 v/v) | 0.6 | 18 | 107 | 0.38 |
| 10% PA6/(HFIP/FA 8/2 v/v) | 0.6 | 18 | 160 | 0.15 |

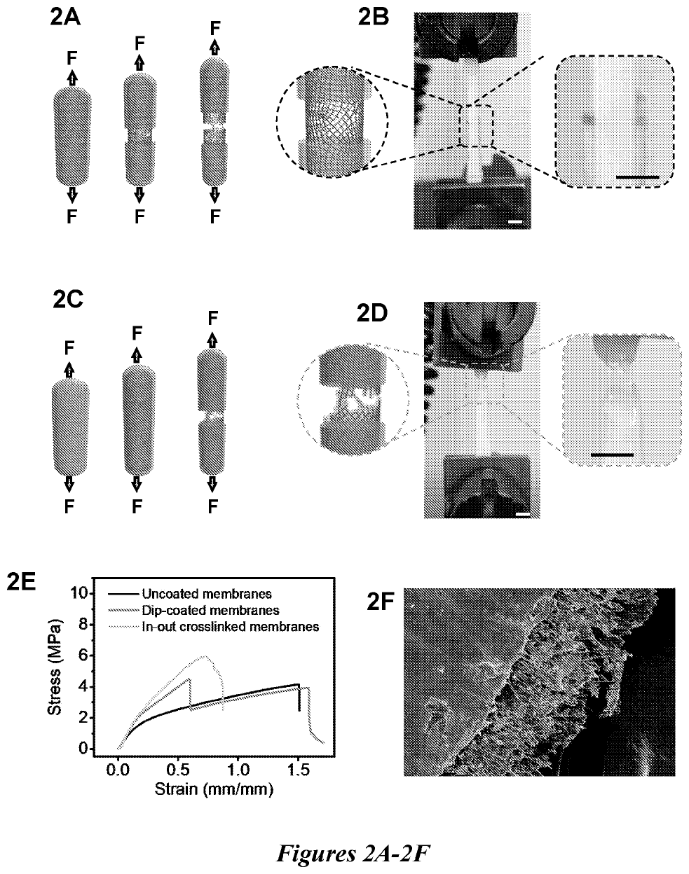

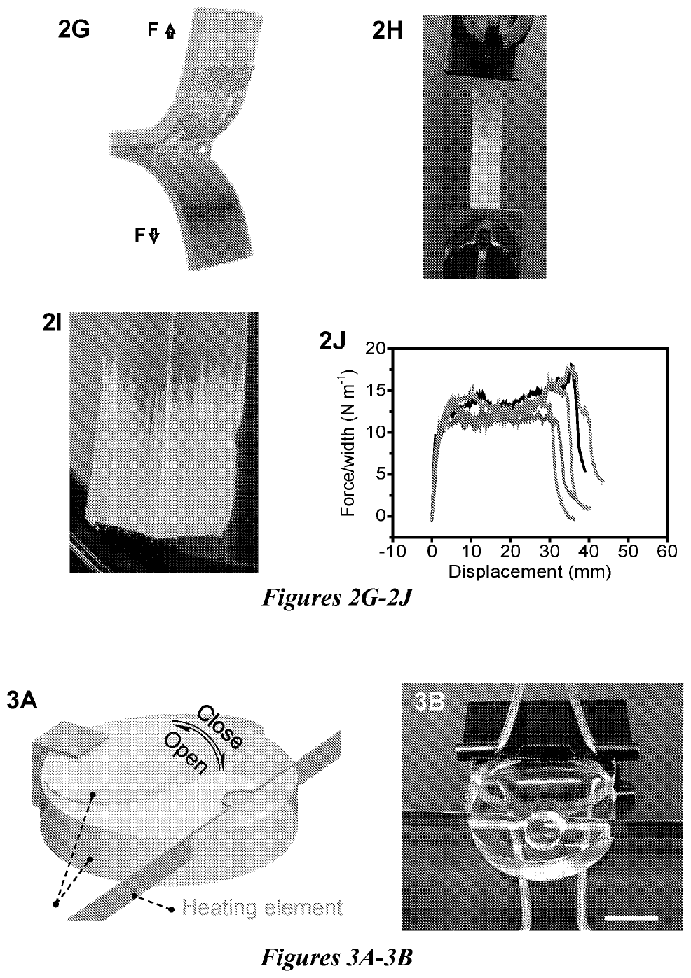

To achieve good reproducibility, not only a highly controllable electrospinning setup is needed, but also stable recipes for the polymer solutions and electrospinning. By using hexafluoroisopropanol (HFIP) and HFIP/FA (8/2, v/v) solvent systems, stable electrospinning of nylon 6 (PA6) solutions was achieved without needle clog, making it possible to fabricate nanofiber tubes with reproducible and controllable quality. As a demonstration, more than 20 cm long nanofiber tubes were fabricated (FIG. 1B). Nanofiber tubes with different diameters were generated by using conductive collecting rods with desired diameters (FIG. 1C). In addition, the thickness of nanofiber tubes was controlled by electrospinning time (FIG. 1D). While the average pore size was tailored by adjusting the diameter of nanofibers (FIGS. 2A and 1F-1J). Furthermore, minimal influence of thickness on the pore size of nanofiber membrane was observed (FIG. 1E). Notably, the pore size has a significant influence in preventing cell penetration (FIGS. 1K-1O). While nylon 6 was used for this study, other polymers (polyurethane, polysulfone, polyacrylonitrile, polyethylene terephthalate, polyvinylidene difluoride, polyacrylamide, poly(ethyl methacrylate), poly(methyl methacrylate), polyvinyl chloride, polyoxymethylene, etc.) compatible with electrospinning are also suitable for the fabrication of SHIELD devices.

To facilitate the removal of nanofiber tubes from rod collectors, a thin layer of sucrose syrup (25 g mL⁻¹) was coated on the rod collectors before electrospinning. After electrospinning, nanofiber tubes were removed and released from rod collectors by soaking in DI water. The sucrose was removed by washing with a large volume DI water three times (at least 10 min each time). Then nanofiber tubes were placed on a clean surface to dry out. To remove the residual solvents, the dry tubes were heated in a vacuum oven (Temperature 60° C., Pressure 27 in. Hg) for 24 hours.

### Example 2—Preparation of Sucrose Syrup

The sucrose syrup was prepared by adding 45 g of sucrose into 18 mL DI water in 50 mL a falcon tube and resulted in a ˜47 mL mixture after dissolution. The mixture was placed in an oven (132° C.) with the cap closed. Shaking was needed every 10 min for 3 times to accelerate the dissolving process. Once all the sucrose was dissolved (indicated by a colorless solution), the solution was kept in the oven (80° C.) for ˜24 hours after removing the cap. Finally, the solution became viscous (˜42 mL) and turned golden brown. Then it was removed from the oven. The solution was stored at room temperature. If precipitation happened, heating the solution in the oven (132° C.) for 30 min would dissolve the precipitated sucrose. A regular sucrose solution displayed a low viscosity similar to water. In contrast, the sucrose syrup was made highly viscous via our protocol so that the sucrose would stay adhered to collecting rods long enough for fabrication. A low viscosity solution would result in discontinuous droplets on collecting rods in a second due to surface tension that would influence the shape of nanofiber tubes and make it difficult to remove the nanofiber tubes.

### Example 3—In-Out Crosslinking of Alginate

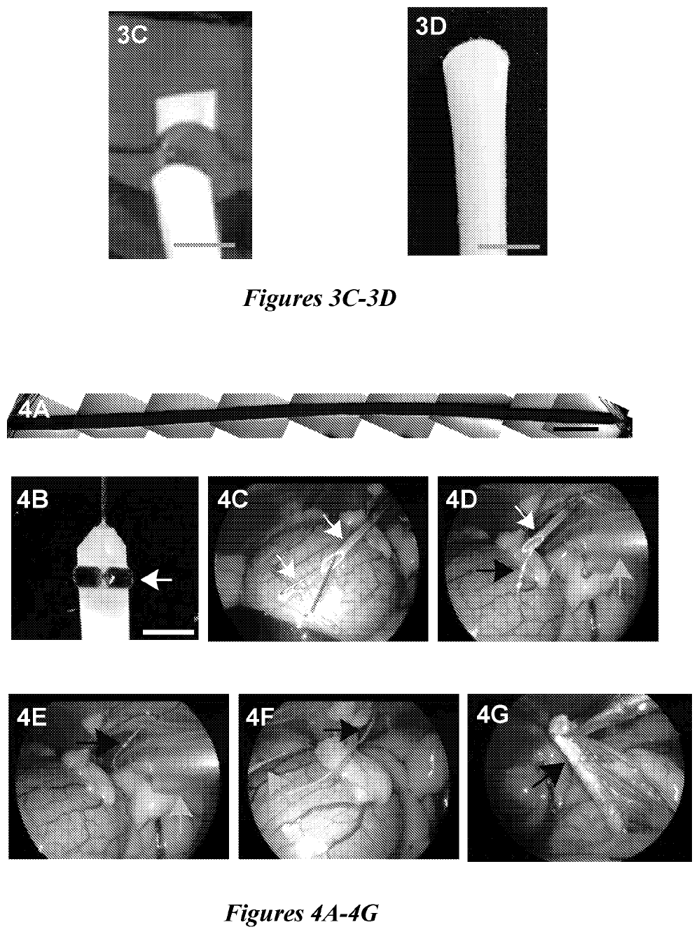

Outer nanofiber tubes (ID 3.2 mm, pore size 1.05 Dry) were cut into sections ˜2.5 cm long, and one end was sealed using the transparent thermo cutter (FIGS. 3A-3D). One-end sealed nanofiber tubes were treated with 20% sodium hydroxide overnight to make them hydrophilic and facilitate the penetration of alginate precursor during in-out crosslinking. After washing away excessive sodium hydroxide, the nanofiber tubes were sterilized by autoclave (120° C., 20 min). Unless otherwise noted, the length, diameter, and average pore size of nanofiber tubes were kept constant at 2.5 cm, 3.2 mm, 1.05 μm, respectively.

During in-out crosslinking, a stainless steel capillary (OD˜2.5 mm) connected to a syringe (filled with crosslinking buffer, 200 mM BaCl₂) was inserted into the one-end sealed nanofiber tube. The nanofiber tube was first dipped into coating alginate precursor, allowing the penetration of alginate precursor into the nanofiber membranes, thus forming alginate hydrogels after crosslinking in the interconnected pores of nanofiber membranes. Next, the nanofiber tube was filled with crosslinking buffer that diffused through the porous membranes of nanofiber tubes and crosslinked alginate. The diffusion time was controlled to achieve alginate hydrogel coating with a desired thickness. Then uncrosslinked alginate precursor was washed away immediately after a specific diffusion time by shaking the stainless capillary with the nanofiber tube in a reservoir filled with saline. Finally, these alginate hydrogel coated nanofiber tubes were further crosslinked (200 mM BaCl₂) and washed with saline at least 6 times to remove residual crosslinkers. It is important not to contaminate the dry nanofiber tubes with crosslinking buffer before soaking them in alginate solution. Otherwise, the penetration of alginate precursor will be prevented, resulting in poor coating adhesion between alginate hydrogels and nanofiber membranes.

### Example 4—Fabrication of SHIELD Devices

Nanofiber tubes (OD 2.2 mm, pore size 1.67 μm, dry) were cut into 2 cm long sections and sterilized by autoclave (120° C., 20 min) to prepare inner nanofibrous tubes. Next, inner nanofibrous tubes were soaked in crosslinkers (5 mM BaCl₂ 95 mM CaCl₂)) for 20 s. In the meantime, cells were mixed with 2% SLG100. Then excessive crosslinkers were removed using sterile napkins. It was crucial to make sure that no visible liquid was left in the lumen. Before applying 80 μL volume cells/SLG100 mixture, one arm of tweezer was inserted into the lumen of an inner nanofibrous tube for rotating when applying cells/alginate precursor around it. Once uniform cell loading was achieved, inner nanofibrous tubes were further crosslinked in the crosslinking solution for 4 min. Then an inner nanofibrous tube was inserted into an outer nanofibrous tube (coated by in-out crosslinking method, ID 3.2 mm, pore size 1.05 μm, length ˜2.5 cm) immediately followed by 6 times of washing. Finally, the open end of the outer nanofibrous tube was sealed with the transparent thermo cutter. In addition, the sealing end was applied with coating alginate precursor and crosslinked in 200 mM BaCl₂ for 30 s. After washing 6 times, the SHIELD devices were imaged and incubated for at least 1 h before implantation. It should be noted that saline was the washing buffer and also used for dissolving alginate for rat islets encapsulation, while saline was replaced with HBSS for encapsulation of human SC-β cells.

Each device's dosage was controlled by dispersing 80 μL, 2% SLG100/islets mixture around the inner nanofibrous tube (600 islet equivalents (IEQs) for rat islets or 4,500 clusters for human SC-β cells). The existence of pre-loaded crosslinkers (95 CaCl₂), 5 mM BaCl₂ in saline) in the pores of inner nanofibrous tubes allowed for the uniform dispersion and in situ crosslinking of alginate/islets mixture in ˜1 min. By making full use of the shrinking (in crosslinkers) property of alginate hydrogels, a typical SHIELD device was achieved by inserting a freshly crosslinked inner nanofibrous tube to a pre-coated outer nanofibrous tube. Post-insertion washing in saline and incubation in medium permitted the equilibration and swelling of alginate hydrogels, which rendered the well-fit SHIELD device having islets distributed in the wall between the inner and outer nanofibrous tubes and thus ensured a short diffusion distance.

### Example 5—Characterizations of SHIELD Devices

Nanofibers were imaged by a field emission scanning electron microscopy (Zeiss-Gemini-500-FESEM). The diameter of nanofibers was determined by analyzing the SEM images using Adobe Acrobat (Adobe, San Jose, Calif.). The pore size of the nanofiber membranes was measured using a capillary flow porometer (PMI, CFP-1100-AEHXL).

### Example 6—Mechanical Test of SHIELD Devices

Mechanical properties were measured using a mechanical testing machine (Instron 5965). Specifically, a tensile test (tensile rate 50 mm min⁻¹, clamping distance 20 mm) was conducted to determine the mechanical properties of nanofiber tubes (diameter 3.2 mm, thickness 140 μm, length 30 mm). For dip-coated samples, the fabrication procedure was quite close to the in-out crosslinking method, except for the timing of injecting the crosslinker. Specifically, the dip-coated samples (FIGS. 2C and 2D) were prepared by injecting crosslinkers first to prevent the penetration of alginate precursor into nanofiber membranes. A peeling test was conducted to determine the coating fidelity (tensile rate 50 mm min⁻¹, clamping distance 20 mm, sample width 10 mm). The samples for the peeling test were prepared by in-out crosslinking with minor modifications. Particularly, only a partial length (˜2 cm) of the devices was soaked in alginate precursor first to allow the alginate penetration. Then the nanofiber tubes were moved ˜2 cm deeper after injecting crosslinkers to have an area without interlocked interaction for clamping. In addition, the coated tubes were cut along the length direction, resulting in a film (width 10 mm) for the peeling test. It should be noted that 3% regular sodium alginate (PROTANAL LF 10/60FT) in saline was used for the tensile and peeling tests and measured directly after crosslinking.

### Example 7—In Vitro Test

NIH3T3/GFP mouse fibroblasts were used for the test of cell escape and cell attachment. NIH3T3 mouse fibroblasts were used for viability test, Live/Dead staining was conducted according to the manufacturer's protocol (ThermoFisher) and imaged using an inverted fluorescent microscope (EVOS fl). All samples were cultured in DMEM supplemented with 10% FBS and 1% P/S. The medium was changed every other day. The culture environment was maintained in a 37° C. incubator with 5% humidified atmosphere of CO₂.

For the cell escape test, cells were dispersed in 20 mg mL⁻¹ fibrinogen/saline and then mixed with 0.5 U mL⁻¹ thrombin, 100 mg mL⁻¹ gelatin/saline in a volume ratio of 1:1 to get a final concentration of 10 mg mL⁻¹ fibrinogen, 0.25 U mL⁻¹ thrombin, 50 mg mL⁻¹ gelatin/saline solution with a cell density of 1 million mL⁻¹. Next, 60 μL cell-matrix suspension was filled into each one-end sealed, coated or uncoated nanofiber tube (length 2.5 cm, diameter 3 mm) using a 1 mL syringe connecting to a 23 G blunt needle, followed by thermo sealing using the transparent thermo cutter. Devices for the cell escape test were imaged on day 2, 5, 7, 10, and 14.

A presto blue assay was conducted on day 2, 3, and 4, respectively. Each sample was incubated with 400 μL 10% presto blue solution in a 500 μL Eppendorf tube for 1 hour. After incubation, 100 μL incubated presto blue solution in triplicate was transferred to a 96 well plate for fluorescence reading. The excitation/emission wavelengths were 560/590 nm. The readings were normalized according to the background reading of 10% presto blue solution incubated without samples.

For the cell attachment test, coated nanofiber tubes were cut along the length direction into films. With the coated surface facing up and fixed by PDMS rings in 6 well plates, 3 mL cell suspension containing 2.5×10⁶ NIH3T3/GFP cells were seeded on the coating surface. After 1 day of incubation, each sample was gently transferred to a fresh medium and was imaged under an inverted fluorescent microscope (EVOS fl).

### Example 8—STZ-Induced Diabetic Mice

Male C57BL/6J mice purchased from Jackson Lab were intraperitoneally injected with 140 mg kg⁻¹ STZ to make them diabetic. The diabetes was confirmed before implantation by at least two consecutive measurements of blood glucose higher than ˜500 mg dL⁻¹. Male SCID-beige mice purchased from Taconic Farms were intraperitoneally injected with 140 mg kg⁻¹ STZ to induce diabetes. The diabetes was confirmed before implantation by at least two consecutive measurements of blood glucose higher than ˜350 mg dL⁻¹.

### Example 9—Islet Isolation

Sprague-Dawley rats obtained from Charles River Laboratories were used for islet isolation. First, the rats were anesthetized by 3% isoflurane in oxygen. Second, rat pancreases were cannulated with 0.16 mg mL⁻¹ liberase (Roche Diagnostics GmbH) dissolved by M199 medium. Third, the pancreases were detached from other organs and collected into 50 mL falcon tubes (2 pancreases per tube) placed in an ice bath. Fourth, the pancreases were digested in 37° C. water bath for ˜30 min. The digestion was stopped by a cold RPMI medium supplemented with 10% FBS and 1% pen strep (purification medium). After vigorously shaking to break pancreases into small pieces, twice more washing with purification medium was conducted. Then pancreases were filtered by a 450 μm sieve. The supernatant was collected and rewashed with the purification medium. Then cells were suspended in 20 mL Histopaque 1077 with 10 mL purification medium on the top and centrifuged at 1700 RCF (0 break and 0 acceleration) for 17 min at 4° C. (repeated twice). Next, the islets were collected from the interface of Histopaque 1077 and purification medium. Islets were further purified by gravity sedimentations and handpicking to remove impurities. Finally, islets were washed once with purification medium and incubated overnight in a low adhesion petri dish with purification medium for further use.

### Example 10—Aggregation of Human SC-β Cells

Human SC-β cells were provided by Novo Nordisk. During the aggregation process, ˜2.2 million mL⁻¹ single cells in re-aggregation medium were first seeded into a 250 mL flask (Corning, #431144). The flask was placed on an orbital shaker (70 rpm) in a 37° C. incubator with 5% humidified atmosphere of CO₂. After 48 hours, the re-aggregation medium was replaced with culturing medium and further cultured for 24 hours. On day 3, the aggregated clusters were harvested for encapsulation.

### Example 11—Mouse Surgeries for Implantation and Device Retrieval

Mice were anesthetized using 3% isoflurane in oxygen. The ventral area was shaved and sterilized by betadine and 70% ethanol. A minimal incision with a length of ˜5 mm was made to implant the devices and was subsequently closed by a suturing process. Retrieval was conducted at different time points. If the blood glucose was under control, a survival procedure was conducted. Blood glucose was then further monitored to confirm that mice were diabetic after retrieval and previous normoglycemia resulted from implanted devices. If the blood glucose was out of control at the endpoint, devices in most mice were retrieved after euthanizing the mice.

### Example 12—Mouse Monitoring and Characterization

Blood glucose and body weight were measured every other day in the first week after implantation and twice a week afterwards. Blood was collected from the tail using a 27 G needle to prick the tail vein and analyzed using a Bayer Contour Next EZ blood glucose meter.

Oral glucose tolerance test (OGTT) was conducted to confirm the functionality of the devices. Specifically, mice were fasted for ˜12 hours before injecting 2 g kg⁻¹ D-glucose per body weight dissolved in tap water at a concentration of 320 mg mL⁻¹. Then blood glucose was measured at 0, 15, 30, 60, 90, 120 min.

When human SC-β cells were encapsulated and transplanted, human C-peptide was quantified by measuring mouse serum from non-fasting mice using ultra-sensitive ELISA kits (Mercodia) according to the supplier's protocol. About 200 μL facial vein blood was collected and clotted naturally for ˜15 min at room temperature. Then the clot was removed by centrifuging at 2000 rpm for 10 min, which resulted in ˜100 μL supernatant of serum.

### Example 13—Characterizations of Retrieved Devices

Retrieved devices were imaged under an optical microscope (EVOS fl) or stereomicroscope (Olympus SZ61) immediately after retrieval. Devices were fixed in 10% neutral buffered formalin and kept in 70% ethanol before being sent for histology. The retrieved devices were embedded in paraffin, sectioned (thickness 10 μm), and stained with H&E or Masson's Trichrome by Cornell Histology Core Facility. The H&E and Masson's Trichrome samples were imaged by a microscope (IN200TC, Amscope). In addition, rat islets were further stained with insulin/glucagon/DAPI. Human SC-β cells were stained with C-peptide/PDX1/DAPI and insulin/glucagon/DAPI. Imaging was conducted by a laser scanning confocal microscope (LSM 710).

The coating stability was qualified by dividing the area of the remaining coated alginate hydrogel after retrieval (according to H&E images) by that of the original alginate hydrogel coating. For uncoated devices, thick cellular overgrowth with a complete coverage was usually found and therefore quantified by measuring the thickness of the fibrotic layer. In contrast, the cellular overgrowth on coated devices was very mild and usually not fully covered by cells, and therefore, it was characterized by the percentage of cell coverage.

Static GSIS was conducted for the retrieved devices using the Krebs Ringer Bicarbonate (KRB) buffer (135 mM NaCl, 3.6 mM KCl, 5 mM NaHCO₃, 0.5 mM NaH₂PO₄, 0.5 mM MgCl₂, 1.5 mM CaCl₂, 10 mM HEPES, 0.1% BSA) supplemented with 2 mM or 20 mM D-glucose. Specifically, each retrieved device was incubated in 2 mM D-glucose KRB buffer for 1 h to equilibrate, then sequentially incubated (1 h) in 2 mM and 20 mM D-glucose KRB buffers. It should be noted that 3 mL buffer was used for the GSIS test. The buffers at 2 mM and 20 mM D-glucose were collected for characterization using rat ultrasensitive insulin ELISA (ALPCO 80-INSRTU-EO1, E10) according to the supplier's protocol.

### Example 14—Fabrication of Devices and Surgeries for Dog Studies

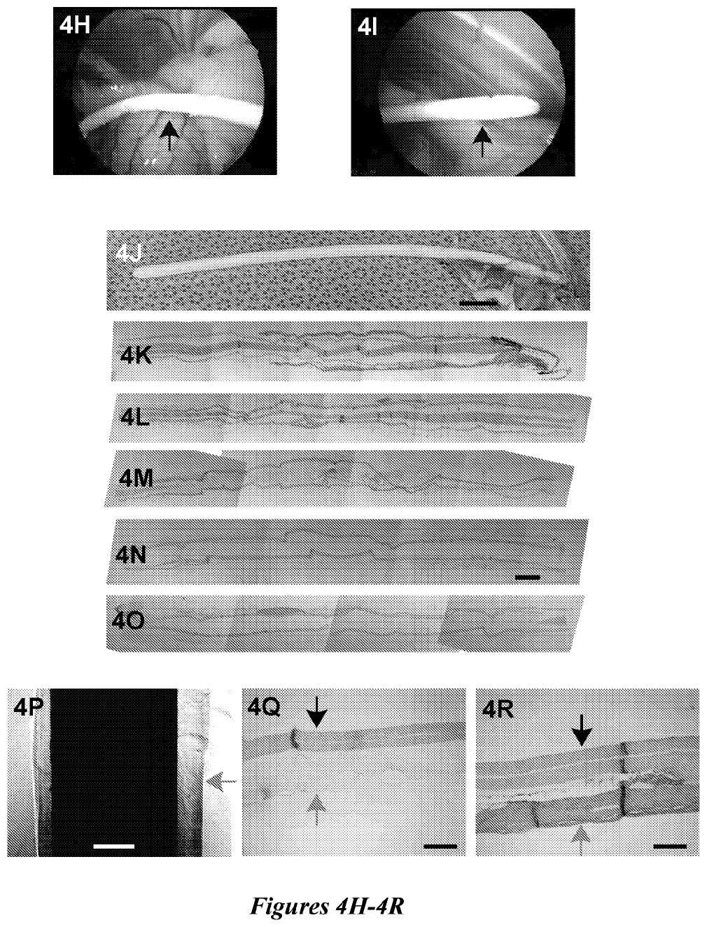

SHIELD devices were adapted to the form of hanging-suture devices. Specifically, a nylon suture and a device with the desired length were bonded together using the transparent thermo sealer (FIG. 4B). In addition, the thermo bonding area and suture were coated with PDMS to mitigate tissue adhesion. The outer nanofibrous tube for hanging suture devices (length ˜12 cm, ID 3.2 mm) was coated by the in-out crosslinking method with 4% modified alginate (3:7). The inner nanofibrous tube (length ˜11 cm, OD˜2.2 mm) was coated with 480 μL 2% SLG100 and inserted into the coated outer nanofibrous tube with the assistance of a stainless-steel capillary. A nylon template (11 cm×2.5 mm×0.25 mm) was inserted into the inner tube to prevent kinking. It should be noted that other plastic membranes or rubbers with similar stiffness to nylon could also be used as the template to prevent kinking. Except for the abovementioned procedures, the fabrication process was the same as the general SHIELD device. Long devices without a hanging suture (length ˜12 cm, ID 3.2 mm) were fabricated with a similar procedure.

Both implantation and retrieval were performed by laparoscopic surgeries. Before implantation, the intraperitoneal space was filled with CO₂ to create enough space for surgical operation. Each device was placed in a plastic tube (˜10 mm in diameter) and delivered through a trocar by pushing with an aluminum rod. The devices were implanted in the area near the bladder. For the hanging-suture SHIELD devices, the suture end was delivered as the head to be caught by a PMI suture grasper (OD 2.1 mm), and the suture was fixed to the recipient's body wall. During retrieval, the mild omentum adhesion was separated by electrocautery. Then the devices were pulled out through a trocar.

### Results of Examples 1-14

Design and Fabrication of SHIELD with Safety, Scalability, and Biocompatibility

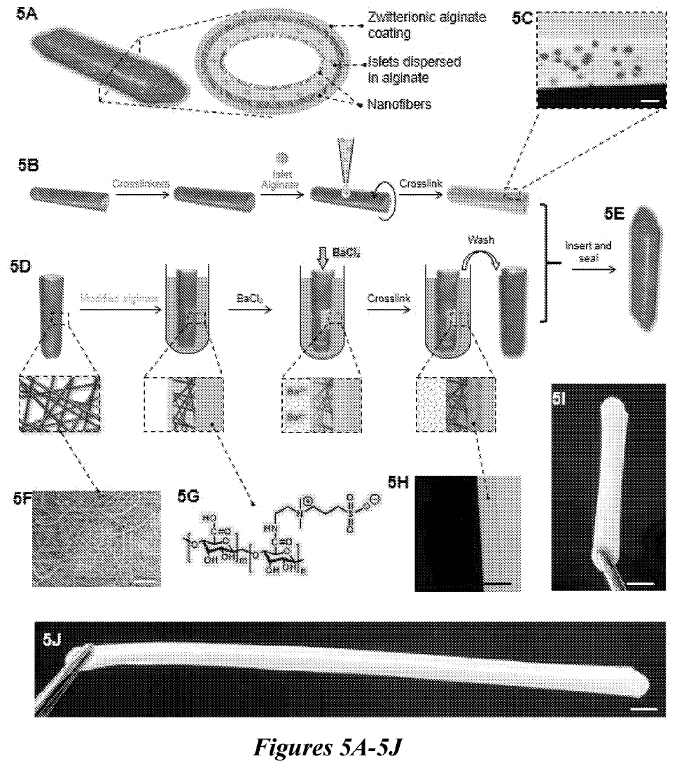

We considered several criteria in designing SHIELD. For scalability, we adopted a concentric geometry and encapsulated cells in the cylindrical wall where the capacity is decoupled from the diffusion distance and increases with the inner diameter of the device (FIGS. 5A-5E), allowing scale-up in both longitudinal and radial directions. For safety, we used electrospun nylon nanofiber membrane as a barrier which is not only mechanically robust but also has tunable pore structures (FIG. 5F), allowing us to balance the safety (i.e. prevention of cell escape) and function (i.e. facile mass transfer). For biocompatibility, we coated the device with the zwitterionically modified alginate to mitigate the fibrotic reactions (FIGS. 5D, 5G, and 5H), thus maintaining facile mass transfer and enabling long-term function of encapsulated cells.

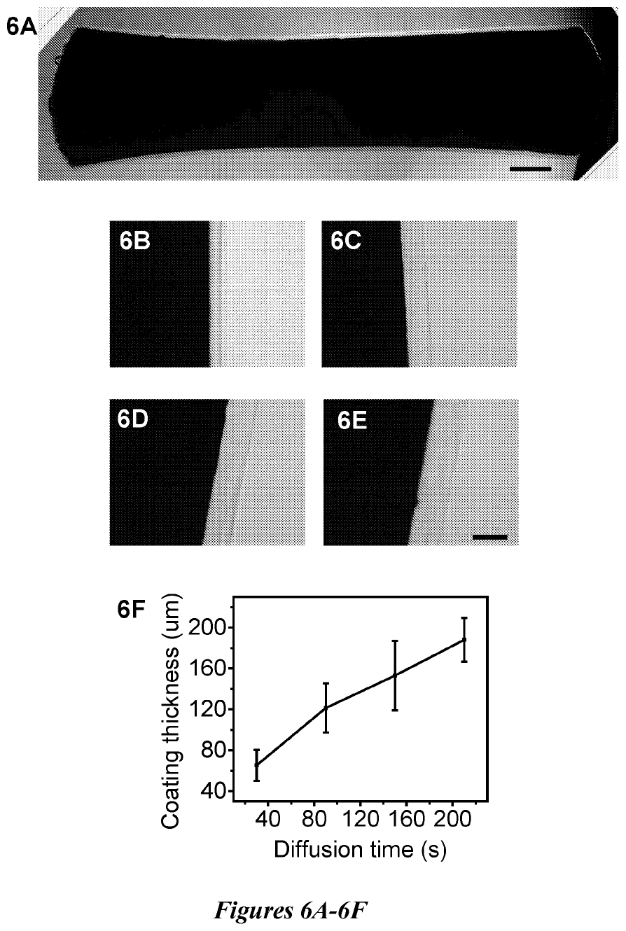

To realize the concentric configuration, we first coated an inner nanofibrous tube with cell-laden alginate hydrogel (FIGS. 5B and 5C), then inserted it into another outer nanofibrous tube (alginate-coated), followed by thermal sealing using a custom-designed thermo cutting device (FIGS. 5D, 5E, and 3A-3D). The alginate coating for the outer tube is critical for the device performance. Previously reported methods such as impregnating porous membranes with alginate precursor and subsequent crosslinking often had poor control over uniformity and thickness (An et al., “Developing Robust, Hydrogel-based, Nanofiber-enabled Encapsulation Devices (NEEDS) for Cell Therapies,” Biomaterials 37:40-48 (2015), which is hereby incorporated by reference in its entirety). Here we developed a new method to achieve a uniform coating with a controllable thickness (FIG. 5D). Specifically, a one-end sealed, dry nanofibrous tube was first dipped into alginate precursor to facilitate the alginate penetration into the nanofibrous wall. Subsequently, a crosslinking solution was injected into the lumen from the open end so that crosslinkers could uniformly diffuse through the interconnected pores of the nanofibrous wall and gradually crosslink the alginate from inside to outside (we term this process as “in-out crosslinking”), resulting in a layer of uniform and smooth hydrogel coating (FIGS. 5H and 6A). The coating thickness could be controlled by adjusting the diffusion time. For example, the coating thickness increased from ˜65±15 μm to ˜188±21 μm when the diffusion time was extended from 30 s to 210 s (FIGS. 6B-6F). After washing away uncrosslinked alginate, the coated tube was further crosslinked to improve the strength of the hydrogel coating. Importantly, the “in-out crosslinking” is applicable for devices with various lengths and diameters (FIGS. 5I and 5J) and scalable to clinically relevant capacities. In addition, the lumen of the inner tube could be filled with a kink-preventing template for long devices, which is crucial for scaling up and will be discussed in the dog study.

The “In-Out Crosslinking” Method Leads to Robust Alginate Coating

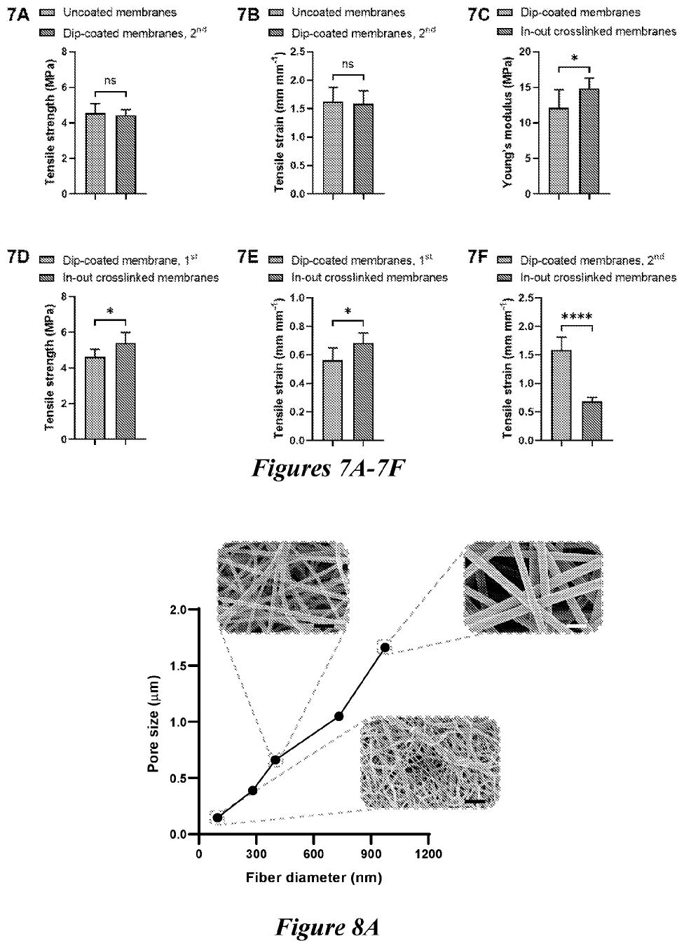

Another advantage of the “in-out crosslinking” is that the interconnected pores of nanofibrous membranes are occupied by alginate hydrogels, enabling a robust mechanical interlock between the coated hydrogel and the membrane, and thus good coating stability. To verify the interlock, we first performed tensile tests (FIGS. 2A-2D). Dip-coated membranes without interlocked interaction (see details in methods) were prepared as a control. It is noted that the thickness of alginate was neglected for the convenience of comparison. According to the stress-strain curve, dip-coated membranes exhibited two breaking points (FIG. 2E). At the strain of ˜0.56 mm mm⁻¹, the first stress drop represented the break of alginate coating. Further elongation resulted in apparent delamination between coated alginate and nanofiber membrane (FIGS. 2A and 2B), which was not observed for the “in-out crosslinked” membranes (FIGS. 2C and 2D). In addition, the stress and strain at the second breaking point were in agreement with those of uncoated nanofiber membranes (FIGS. 7A and 7B), further verifying that there was no interlocked interaction. On the contrary, only one breaking point was observed for the “in-out crosslinked” membranes featuring larger Young's modulus than that of dip-coated membranes (FIG. 7C). Furthermore, the tensile strength of the “in-out crosslinked” membranes was significantly higher than that of dip-coated membranes at the first breaking point (FIG. 7D). The tensile strain of the “in-out crosslinked” membranes was between the two breaking points for dip-coated membranes (FIGS. 7E and 7F). These results indicate that the “in-out crosslinking” resulted in the formation of an integrated nanofiber-hydrogel composite. To view the structure of “in-out crosslinked” membranes, scanning electron microscopy (SEM) was used to image the cross-section of lyophilized samples. As expected, the interpenetration between alginate and nylon nanofibers was observed (FIG. 2F). Lastly, to more directly measure adhesion between the hydrogel coating and the nanofibrous membrane, we performed peeling tests (FIGS. 2G-2J). Results showed that the adhesion between coated hydrogel and nanofiber membranes was 13.1±1.5 N m⁻¹ (FIG. 2J), which was remarkable given the intrinsically weak mechanical properties of alginate hydrogels. The strong adhesion was also evidenced by the existence of residual nanofibers on the hydrogel after the peeling (FIGS. 2H and 2I). Taken together, it was clear that the new “in-out crosslinking” method resulted in a uniform and robust alginate coating with a controllable thickness and a strong adhesion.

Optimize the Pore Size by Balancing Safety and Mass Transfer

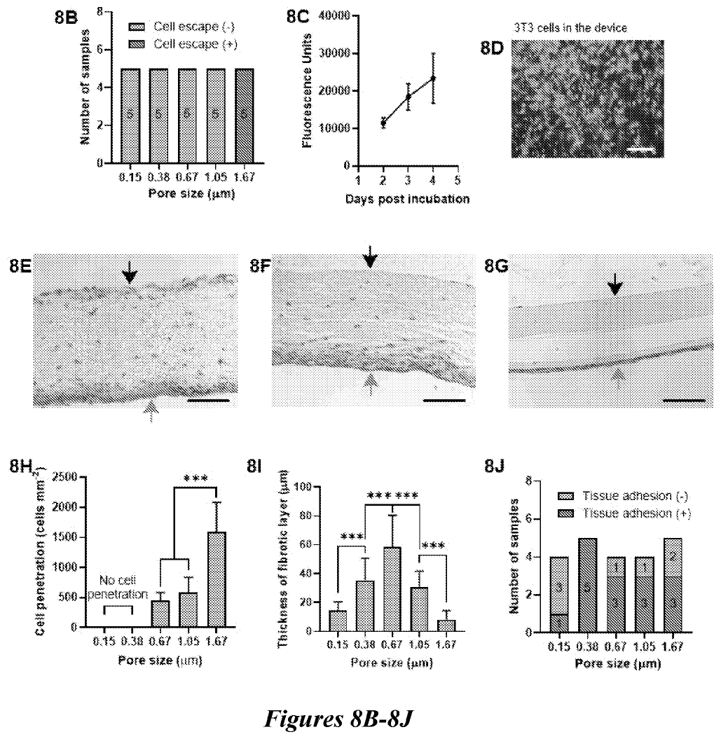

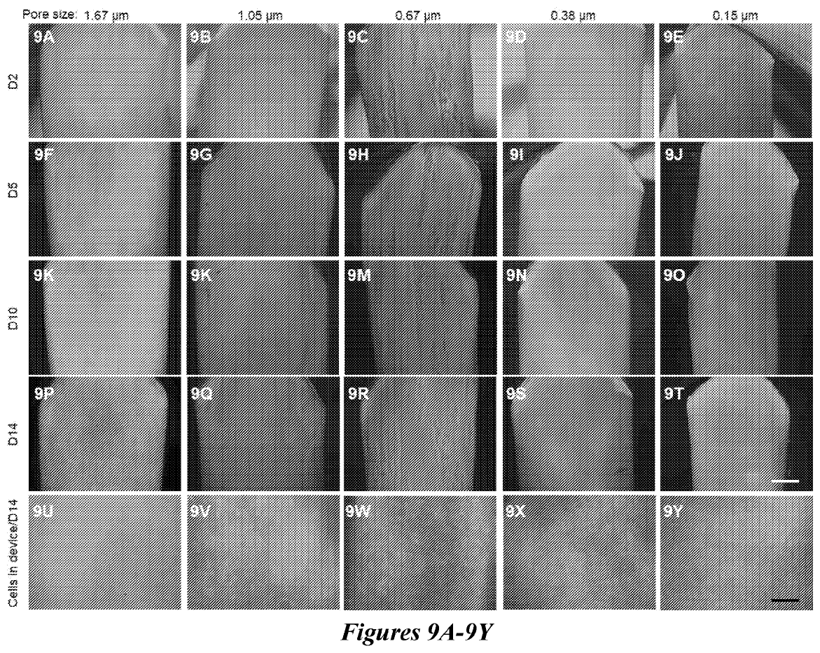

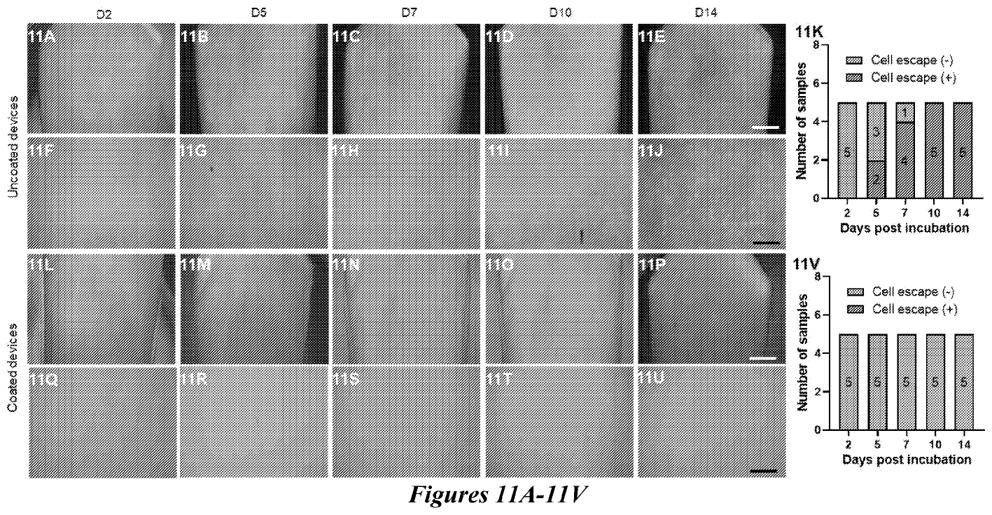

Next, we sought to optimize the mass transfer while ensuring that the SHIELD device could confine encapsulated cells and prevent cell escape. Devices with average pore sizes ranging from 0.15 μm to 1.67 μm were fabricated by adjusting the nanofiber diameter (FIGS. 8A and 1A-1J). To investigate cell escape, GFP expressing cells (NIH3T3/GFP) dispersed in 60 tit fibrin/gelatin hydrogels at a density of 1.0 million mL⁻¹ were encapsulated in the device, cultured, and monitored for up to 2 weeks. Fibrin gel, which could be degraded by NIH3T3/GFP cells in 2-3 days, was used as the matrix to allow for free cell growth and migration. Both uncoated and coated devices were evaluated. Cell escape was only detected for the uncoated device with the pore size of 1.67 μm (FIGS. 8B, 9A-9T, 10A-10T, and 11A-11V): 2 out of 5 devices failed to confine the cells since day 5 and the rest 3 from day 7 to day 10 (FIGS. 11A-11K). All other devices completely confined the cells despite massive cell growth inside (FIGS. 9A-9Y, 10A-10Y, and 11A-11V).

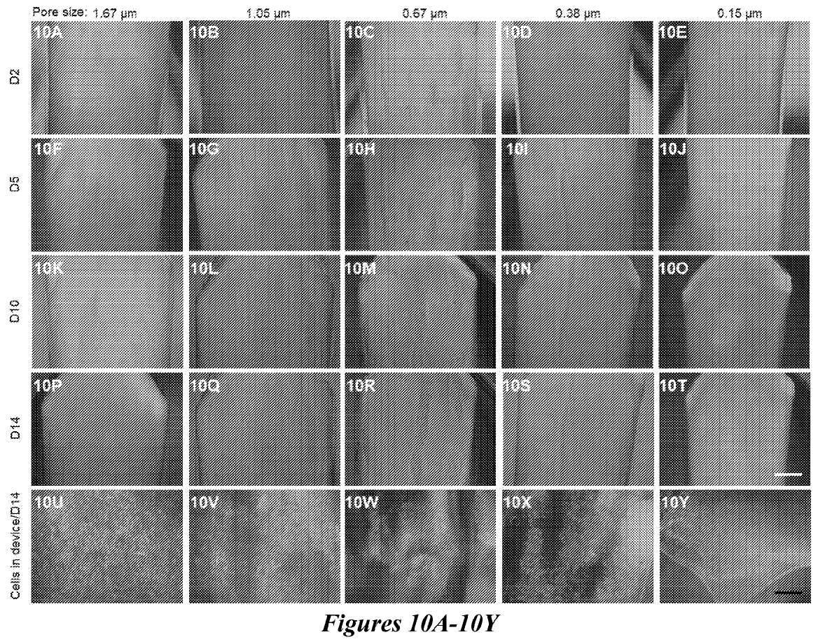

It is interesting to note when the devices were coated with alginate hydrogel, even the 1.67 μm ones could prevent cell escape (FIGS. 10A-10V and 11L-11V), suggesting that the formation of alginate hydrogel in the interconnected pores of nanofiber membranes could stop the cells from escaping. Importantly, massive and crowded cells were found in both coated and uncoated devices for all the pore sizes investigated (FIGS. 9U-9Y and 10U-10Y). Furthermore, presto blue and Live/Dead staining confirmed that cells remained viable and proliferated normally in the coated devices, verifying that the mass transfer of the SHIELD device was sufficient for encapsulated cells (FIGS. 8C and 8D). Lastly, we implanted empty uncoated devices into the intraperitoneal cavity of C57BL6/J mice for two weeks to assess the fibrotic reaction and penetration of host cells. Histological images (FIGS. 8E-8H and 1K-1O) revealed that the 1.67 μm device allowed extensive cell penetration into the nanofibrous membrane while the other devices had minimal (for 0.67 μm and 1.05 μm devices) or no cell penetration (for 0.15 μm and 0.38 μm devices). In addition, the thickness of the fibrotic layer on the device first increased and then decreased when the average pore size was changed from 0.15 μm to 1.67 μm, with the peak at 0.67 μm (FIG. 8I). Tissue adhesions occurred for all pore sizes, with the highest frequency for 0.38 μm devices (FIG. 8J). Considering all these results, we chose the device with 1.05 μm average pores in following investigations to maximize the mass transfer while ensuring no cell escape and minimizing cell penetration, fibrotic deposition, and tissue adhesion. It is noted that these in vivo tests were performed based on uncoated devices; alginate coating would provide additional protection and significantly improve the biocompatibility.

Stable Zwitterionic Alginate Coating Results in Superior Biocompatibility

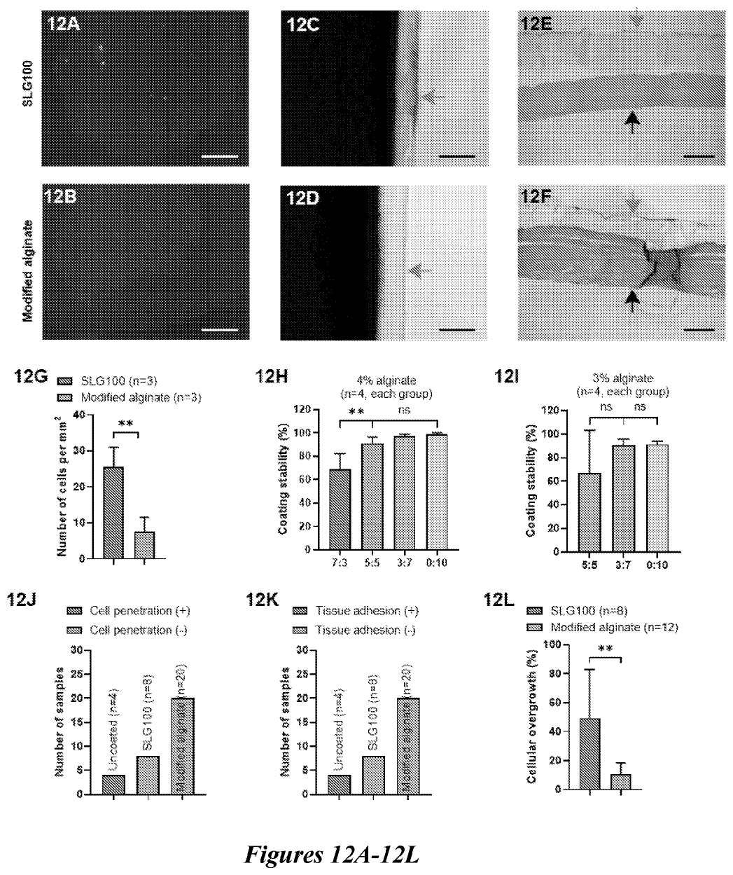

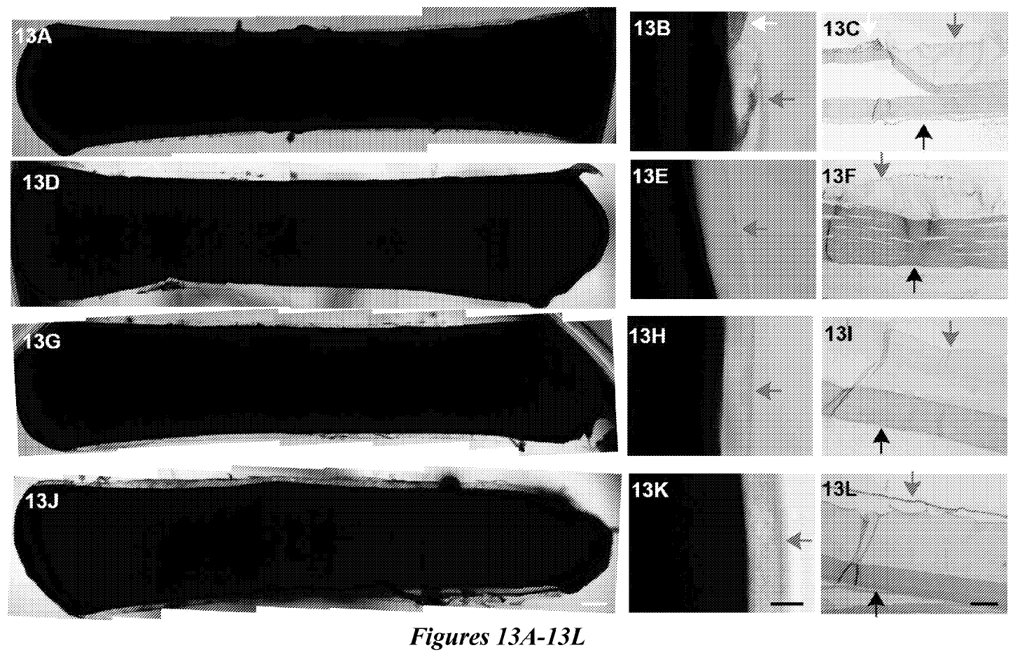

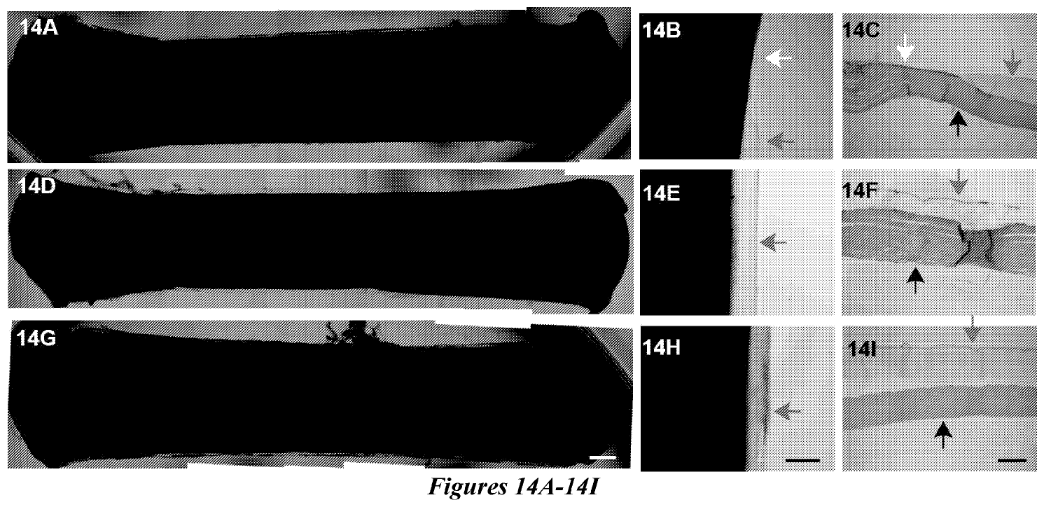

Alginate hydrogel is a commonly used material for cell encapsulation. However, its inadequate biocompatibility remains a challenge. We previously developed zwitterionically modified alginates and showed reproducible and robust reduction of cellular overgrowth on microcapsules in mice, dogs, and pigs. Here we applied one of the zwitterionic alginates sulfobetaine-modified alginate (SB-alginate) to the SHIELD device as a thin and uniform coating to improve its biocompatibility. By seeding NIH3T3/GFP cells on the outer surface of coated devices, we confirmed that modified alginate indeed performed better in preventing cell attachment compared to unmodified SLG100 alginate (FIGS. 12A, 12B, and 12G). To obtain the best coating stability in vivo, we used the “in-out crosslinking” method, and tried alginate with three different ratios between SB-alginate and unmodified high molecular weight alginate SLG100 (i.e. SB-alginate:SLG100=7:3, 5:5, 3:7). Neat SLG100 alginate coating (or 0:10) was included as a control. After intraperitoneal implantation in C57BL6/J mice for 2 and 4 weeks, devices were imaged right after retrieval and then processed with histological sectioning and H&E staining (FIGS. 12C-12F). At a total alginate concentration of 4%, the coating was relatively unstable when the ratio was 7:3, with ˜69% coated hydrogel remaining on the device after retrieval based on H&E images. However, the other two ratios (5:5, 3:7) had more than 90% coated hydrogel remaining, which was comparable to that of neat SLG100 (i.e. 0:10) coating (FIGS. 12H and 13A-13L). Since a lower concentration is expected to provide better mass transfer, we further tested 3% alginate concentration with 5:5, 3:7, and 0:10 ratios. While the 5:5 ratio resulted in relatively unstable coating with a large variation (˜67% coated hydrogel remaining), the 3:7 and 0:10 ratios led to much more robust coatings (˜90% coated hydrogel remaining, FIGS. 12I and 14A-14I).

Notably, even with some hydrogel detachments (5:5 at 3% and 7:3 at 4%), cell penetration was not observed in the detached areas (FIGS. 12J, 13C, and 14C) probably due to the formation of hydrogel within the interconnected pores, confirming that the coating and the nanofiber membranes provided double protections. In addition, tissue adhesion was not observed for any coated devices (20 for modified alginate coating and 8 for neat SLG100 coating), including those having alginate detachment, suggesting the excellent biocompatibility of SB-alginate hydrogels (FIG. 12K). More importantly, the modified alginate coatings (both 3:7 at 3% and 5:5, 3:7 at 4%) exhibited significantly less cellular overgrowth than the neat SLG 100 (FIGS. 12C-12F, 12L, 13A-13I, and 14A-14I), consistent with the results we observed for alginate microcapsules (Liu et al., “Zwitterionically Modified Alginates Mitigate Cellular Overgrowth for Cell Encapsulation,” Nat. Commun. 10:1-14 (2019), which is hereby incorporated by reference in its entirety). In general, the coating with either neat SLG100 or modified alginate prevented the devices from being fully covered by cellular overgrowth. In contrast, uncoated devices were usually entirely covered by a layer of cellular overgrowth with varying thicknesses (FIGS. 1K-1O). In particular, the devices coated with modified alginate hydrogels had a much smaller percentage of cellular coverage (˜11%) relative to those coated with neat SLG100 (˜50%) (12C-12F, 12L, 13A-13L, and 14A-14I). Besides, the cellular overgrowth was usually thin, with only one or two layers of cells.

SHIELD Supports Long-Term Function of Rat Islets in C57BL6/J Mice

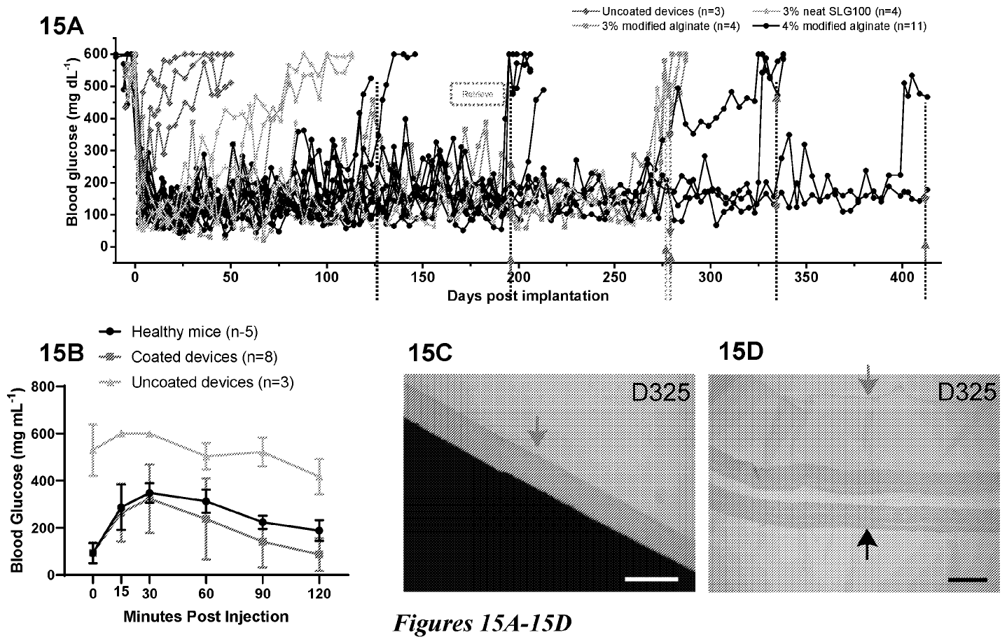

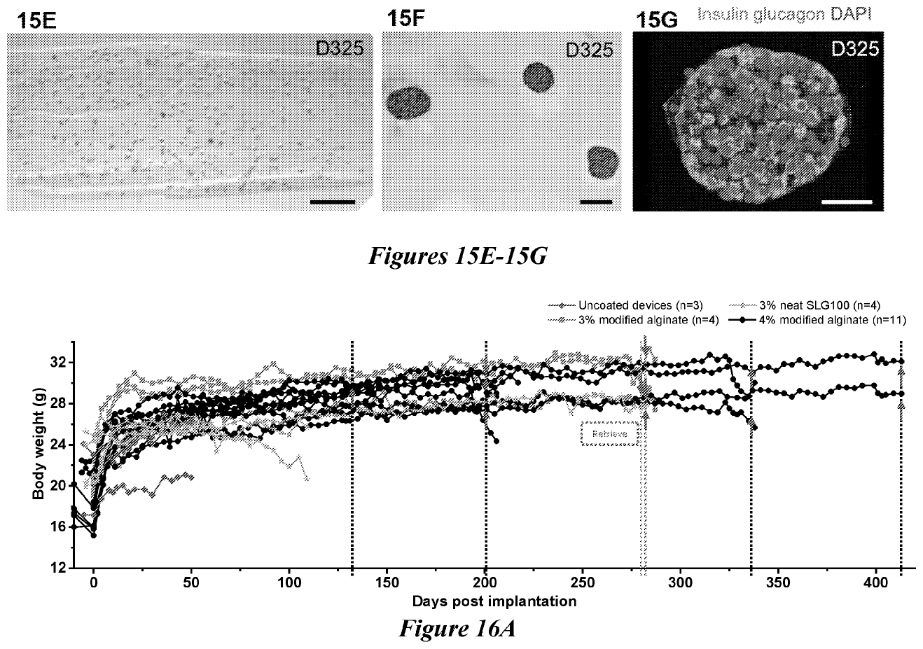

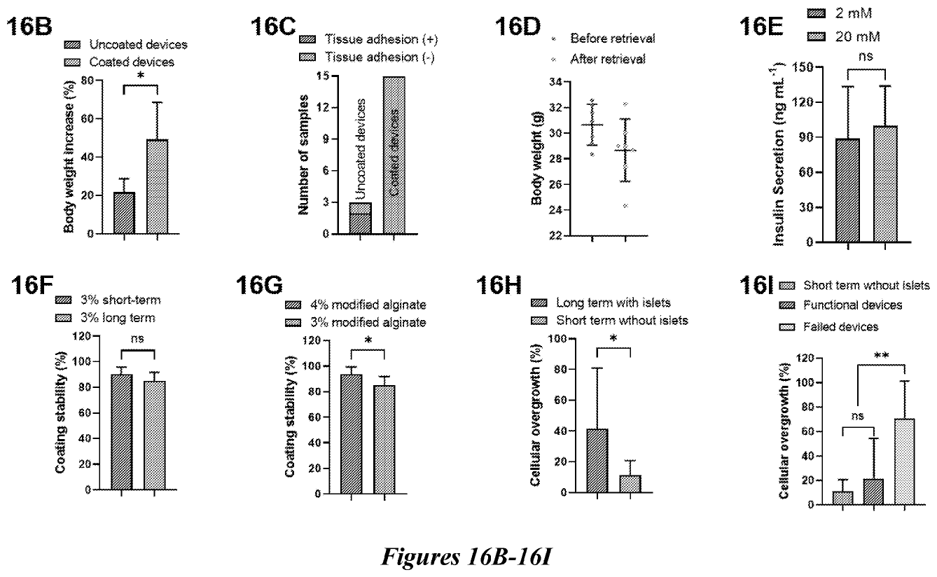

To evaluate the efficacy of the SHIELD device, we encapsulated rat islets (600 islet equivalents (IEQs)) and transplanted them in the intraperitoneal space of streptozotocin (STZ) induced C57BL6/J diabetic mice. Devices coated with 3% (n=4) and 4% (n=11) modified alginate both at 3:7 ratio were investigated; devices coated with 3% neat SLG100 (n=4) and uncoated devices (n=3) were included as controls. According to the blood glucose data, the devices coated with modified alginate showed much better performance than those coated with neat SLG100 or uncoated devices (FIG. 15A). Although all mice treated with devices became normoglycemic shortly after transplantation (20 mice in 2 days, 1 mouse in 4 days), mice treated with uncoated devices maintained a short period of normoglycemia and all returned the diabetic state within 12 days. In contrast, normoglycemia was greatly extended when devices were coated with alginate. With the neat SLG100 coating, 2 out of 4 devices failed on day 35 and 63, 1 was functional on day 85 when the mouse was found dead for an unknown reason, and 1 was functional when retrieved on day 270. With the modified alginate coating, only 1 out of 15 devices failed within 100 days (at day 71), 2 failed between 100 and 200 days, 3 failed between 200 and 300 days, and 7 were still functional when retrieved for up to 399 days (1 mouse died during the study, and 1 had spontaneous reverse of diabetes; detailed information about all mice is summarized in Table 2). At ˜50 days after implantation, the body weight increase for the modified alginate coated device group (˜49%) was significantly higher than that for the uncoated group (˜22%) (FIGS. 16A and 16B), indicating the better performance of modified alginate coated devices.

TABLE 2
The performance of all SHIELD devices encapsulating rat islets in C57BL6/J mice.

| | | Cure | Cure | | | |
|---|---|---|---|---|---|---|
| | | starting | length | Retrieval | BG after | |
| | Mouse# | date | (days) | date | retrieval | Notes |
| Uncoated | #1 | 2 | 4 | 50 | | Euthanized |
| devices | #2 | 2 | 8 | 50 | | Euthanized |
| (n = 3) | #3 | 2 | 12 | 50 | | Euthanized |
| 3% neat | #4 | 2 | 35 | 78 | Became higher | |
| SLG100 | #5 | 2 | 63 | 78 | Became higher | |
| (n = 4) | #6 | 2 | 85 | 85 | | Found dead with an |
| | | | | | | unknown reason |
| | #7 | 2 | 270 | 270 | Became higher | |
| 3% | #8 | 2 | 71 | 82 | | Euthanized |
| modified | #9 | 2 | 260 | 274 | Became higher | |
| alginate | #10 | 4 | 274 | 274 | Became higher | |
| (n = 4) | #11 | 2 | 274 | 274 | Became higher | |
| 4% | #12 | 2 | 34 | 34 | | Found dead with an |
| modified | | | | | | unknown reason |
| alginate | #13 | 2 | 111 | 124 | | Euthanized |
| (n = 11) | #14 | 2 | 119 | 129 | Became higher | |
| | #15 | 2 | 193 | 193 | Became higher | |
| | #16 | 2 | 193 | 193 | Became higher | |
| | #17 | 2 | 193 | 193 | Became higher | |
| | #18 | 2 | 205 | 217 | | Euthanized |
| | #19 | 2 | 267 | 325 | | |
| | #20 | 2 | 325 | 325 | Became higher | |
| | #21 | 2 | 399 | 399 | Became higher | |
| | #22 | 2 | | 399 | No change | False-diabetic |

To verify the function of implanted devices, oral glucose tolerance tests (OGTT) were performed at various time points (day 50 for mice receiving uncoated devices, day 273 for those receiving devices coated with 3% modified alginate, and day 192, 342, 398 for those receiving devices coated with 4% modified alginate). Results showed a similar glucose clearance profile between the modified alginate coated device group and the healthy control group. In contrast, only a slight blood glucose decrease was observed for the mice treated with uncoated devices (FIG. 15B). Importantly, all modified alginate coated devices (n=15) were retrieved with a tiny incision and without any tissue adhesion, while uncoated devices (2 out of 3) had tissue adhesion issues at retrieval (FIG. 16C). After retrieval of engrafted devices, blood glucose increase and body weight decrease (˜1.4 g in ˜2 weeks) were generally observed, confirming that the normoglycemia was attributed to the therapeutic function of the implanted devices (FIGS. 15A and 16D). Furthermore, ex vivo GSIS test was performed for the retrieved devices. A fair amount of insulin secretion was detected, indicating the function of encapsulated islets in the devices after long-term implantation (FIG. 16E). Imaging of islets harvested from retrieved devices indicated that most islets remained healthy with a round shape and rare necrosis (FIG. 15E). The H&E images and insulin/glucagon staining also confirmed the intact islet morphology and function (FIGS. 15F and 15G).

We further evaluated the coating stability and cellular overgrowth of the retrieved devices. Comparing to short-term (2-4 weeks) studies, coating stability of 3% modified alginate (3:7) seemed to decline slightly after long-term studies (82 days (n=1) and 274 days (n=3)), but it was not statistically significant (FIG. 16F). In particular, the only device that failed within 100 days (at day 71) had hydrogel detachment (with 77% left) and elevated fibrosis. In contrast, 4% alginate hydrogel coating exhibited a comparable long-term and short-term stability, significantly better than the long-term stability of 3% alginate hydrogel coating (FIG. 16G). As expected, long-term implantation with islets resulted in increased cellular overgrowth (˜38% coverage) compared to that of short-term without islets (˜11% coverage) (FIG. 16H). In particular, up to 80% coverage of cellular overgrowth was observed on devices (4 out of 6) that had failed before retrieval (FIG. 16I). However, most functional devices had minimal cellular overgrowth (˜10% coverage) (FIGS. 15C, 15D, and 16I).

SHIELD Supports Long-Term Function of Human SC-β Cells in SCID-Beige Mice

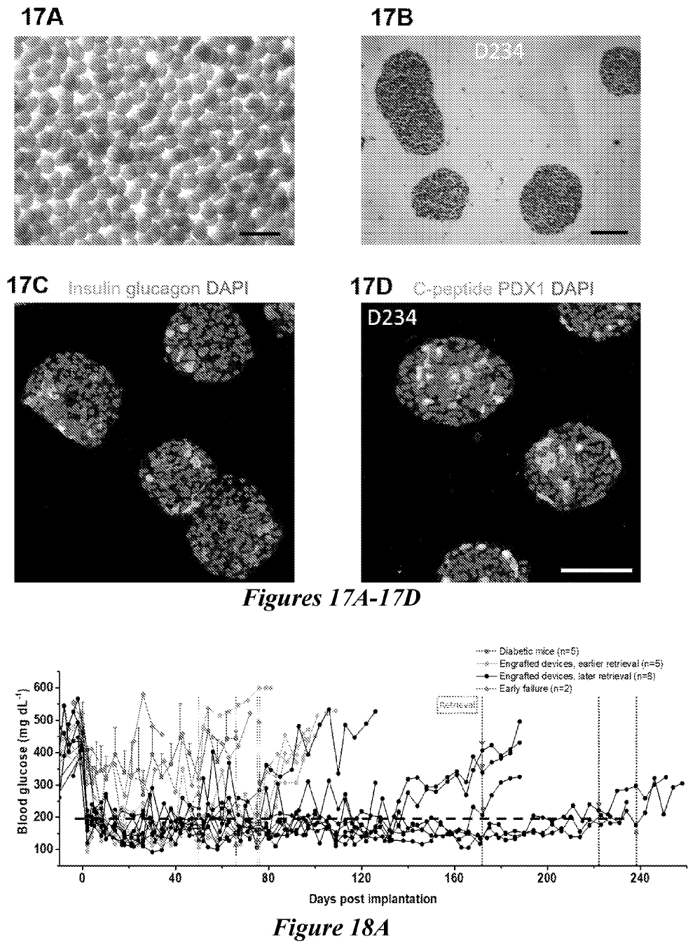

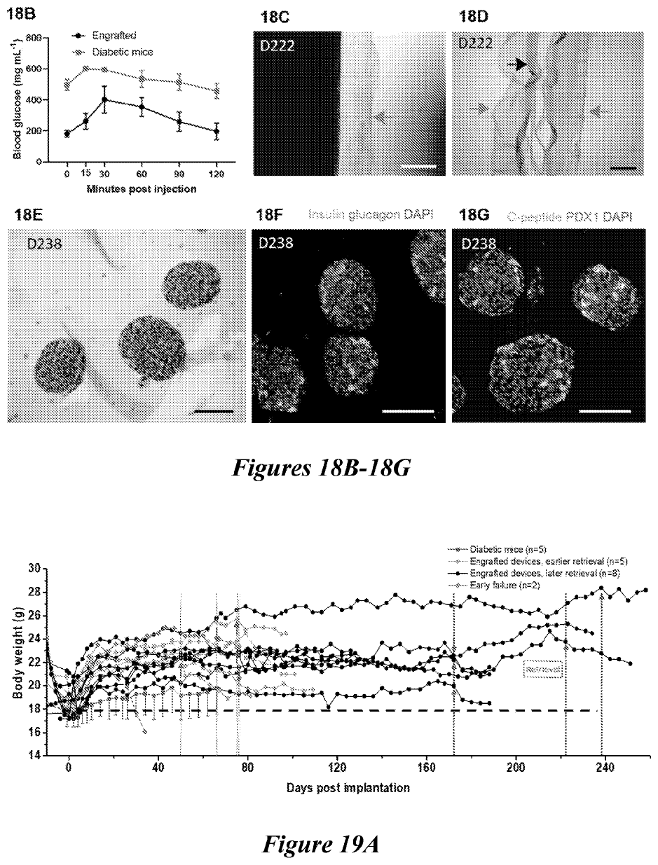

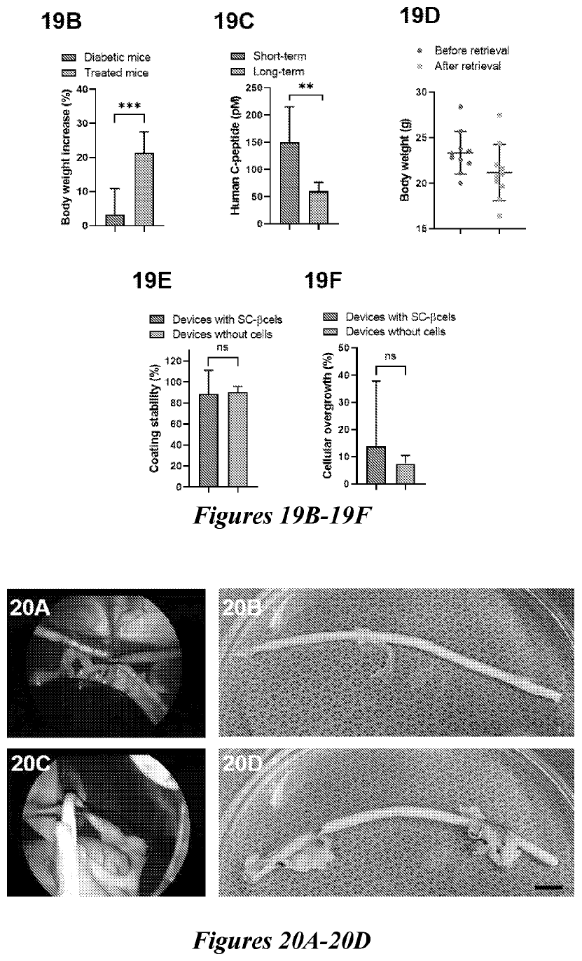

The most impactful application of a safe, scalable, and long-term functional encapsulation device is to deliver human SC-β cells. To test the feasibility, we encapsulated human SC-β cells and transplanted them into STZ-induced immunodeficient SCID-Beige mice. Uniform clusters (˜150 μm) of SC-β cells were prepared by aggregation of single cells (FIG. 17A), and 3% modified alginate (3:7) was used for the device coating. Each mouse was transplanted with a device encapsulating approximately 4,500 clusters. Most devices (13 out of 15) corrected diabetes shortly (within 2-5 days) after implantation and remained functional for up to 238 days (FIG. 18A). Among the functional devices, only 1 failed within 100 days, and 3 failed between 100 and 200 days (detailed information about all mice is summarized in Table 3). At ˜50 days after implantation, body weight increased by ˜22%, significantly higher than that of the diabetic control group (FIGS. 19A and 19B). OGTT tests revealed that the treated mice were significantly better than untreated diabetic control in glucose clearance, confirming the function of encapsulated SC-β cells (FIG. 18B).

TABLE 3
The performance of all SHIELD devices encapsulating
human SC-β cells in SCID-beige mice.

| | | Cure | Cure | | | |
|---|---|---|---|---|---|---|
| | | starting | length | Retrieval | BG after | |
| | Mouse# | date | (days) | date | retrieval | Notes |
| 3% | #1 | 2 | 0 | 36 | | Didn't work, |
| modified | | | | | | euthanized |
| alginate | #2 | 2 | 0 | 76 | | Didn't work, |
| (n = 4) | | | | | | euthanized |
| | #3 | 5 | 50 | 50 | Became higher | |
| | #4 | 2 | 66 | 66 | Became higher | |
| | #5 | 2 | 76 | 76 | Became higher | |
| | #6 | 2 | 76 | 76 | Became higher | |
| | #7 | 9 | 76 | 76 | Became higher | |
| | #8 | 2 | 86 | 138 | | Euthanized |
| | #9 | 2 | 132 | 138 | | Euthanized |
| | #10 | 2 | 140 | 172 | Became higher | |
| | #11 | 2 | 148 | 172 | Became higher | |
| | #12 | 2 | 172 | 172 | Became higher | |
| | #13 | 2 | 222 | 222 | Became higher | |
| | #14 | 2 | 234 | 234 | | Died accidentally |
| | | | | | | due to blood |
| | | | | | | collection |
| | #15 | 2 | 238 | 238 | Became higher | |

Human C-peptide was quantified by measuring its concentration in mouse serum using an ELISA kit. Results confirmed that implanted SC-β cells secreted human C-peptide in all treated mice for both short-term and long-term studies (FIG. 19C). Although the amount of C-peptide seemed to decrease over time, the fact the human C-peptide was detected after 234 days of implantation indicated the potential of this device for SC-β cell encapsulation. To further verify the function of implanted devices, mice were kept alive after retrieving the devices. After device retrieval, blood glucose increased, and body weight decreased for all mice (FIGS. 18A and 19D), confirming that the restoration of normoglycemia was due to the implanted devices. We attributed the success of the devices to the stability and superior biocompatibility of the modified alginate coating. Overall, all devices (n=15) were free of tissue adhesion, and the alginate hydrogel coating for most devices (13 out of 15) remained stable, comparable to the devices from short-term studies (FIG. 19E). Despite a high density of encapsulated human SC-β cells, the cellular overgrowth on most devices (14 out of 15) in SCID-beige mice was as mild as observed during short-term implantation in C57BL6/J mice (FIGS. 18C, 18D and 19F). Most cell clusters in retrieved devices were healthy and functional, containing C-peptide/insulin-positive cells with PDX1 expression as well as glucagon-positive cells (FIGS. 17B-17D and 18E-18G).

Scalability and Retrievability of SHIELD in Dogs

The scalability of an encapsulation device is highly desirable for clinical applications (An et al., “Designing a Retrievable and Scalable Cell Encapsulation Device for Potential Treatment of Type 1 Diabetes,” Proc. Natl. Acad. Sci. USA 115(2):E263-E272 (2017), which is hereby incorporated by reference in its entirety). SHIELD can be scaled up in both radial and longitudinal directions without affecting the diffusion distance. As a proof-of-concept for scalability and retrievability, long devices (length ˜12 cm, ID 3.2 mm) were fabricated and intraperitoneally implanted into healthy dogs (n=3). Considering the occasional coating detachment for 3% modified alginate (3:7), 4% modified alginate (3:7) was used for the dog experiment. Uniform coating along the entire length of the device was achieved using the “in-out crosslinking” method (FIG. 4A). To prevent kinking which may happen for long devices, a nylon ribbon was inserted into the inner lumen to ensure a stable shape while maintaining flexibility. The slender geometry allowed us to implant the device using a minimally invasive laparoscopic procedure. Among the 3 dogs, 1 was implanted with the device without any anchoring. In the other two dogs, the devices were anchored to the body wall through a nylon suture for rapid localization and convenient retrieval. The suture was bonded to one end of the device using a transparent thermo sealer and silicone coating (FIG. 4B). In addition, the suture extended ˜10 cm away from the peritoneal wall to provide freedom for the device movement and minimize irritation to surrounding tissues (FIGS. 4C-4F).

Devices were retrieved after 1 month using a similar laparoscopic procedure. No adhesion to any organs occurred for all three devices except mild adhesion to omentum which could be easily separated by electrocautery (FIGS. 4G-4I, and 20A-20D). Notably, for one of the suture-anchored devices, omentum adhesion only occurred to one end near the anchoring point and the rest was free of adhesion (FIGS. 4G-4K), indicating an excellent performance of the modified alginate coating. Optical images and H&E staining indicated that most of the device was still covered with alginate hydrogels (FIGS. 4K-4Q). Except for the adhesion end (FIGS. 4K and 4R), cellular overgrowth was minimal and comparable to the device in mice (FIGS. 4L-4Q), indicating the excellent biocompatibility of modified alginate hydrogels. These results demonstrate that the device can be scaled up, implanted, and retrieved using minimally invasive procedures.

### Discussion of Examples 1-14

Cell encapsulation has the potential to provide a compliance-free, immunosuppression-free treatment for T1D. However, developing a device that simultaneously meets the requirements of safety, scalability, and long-term functionality is a great challenge. One of the major hurdles is the foreign body response against the encapsulation material. Cellular overgrowth and fibrotic deposition diminish the transfer of oxygen and nutrients to the cells, and insulin and metabolic wastes from the cells. Recent clinical trials using the ViaCyte device and the BetaAir device, two of the most developed ones in the field, have convincingly shown the foreign body response as the critical barrier for function (Bose et al., “A Retrievable Implant for the Long-term Encapsulation and Survival of Therapeutic Xenogeneic Cells,” Nature Biomedical Engineering 4:814-826 (2020); Pullen, L. C., “Stem Cell—Derived Pancreatic Progenitor Cells Have Now Been Transplanted into Patients: Report from IPITA 2018,” Am. J. Transplant 18:1581-1582 (2018); Liu et al., “Zwitterionically Modified Alginates Mitigate Cellular Overgrowth for Cell Encapsulation,” Nat. Commun. 10:1-14 (2019), which are hereby incorporated by reference in their entirety). Alginate hydrogels, either microcapsules or fibers, have shown promising biocompatibility in animal studies. Further chemical modifications can drastically improve its biocompatibility, significantly reducing the foreign body response-induced cellular overgrowth and fibrosis. However, hydrogels are intrinsically weak and easy to swell or break, posing a safety concern for clinical applications, especially when SC-β cells are transplanted.

As reported herein, SHIELD combines the safety of a retrievable device and the biocompatibility of a zwitterionically modified alginate. There are several innovative features in the device design worth reiterating. First, the device has a concentric configuration with cells encapsulated in the cylindrical wall. Compared with previously reported tubular or fiber devices (An et al., “Designing a Retrievable and Scalable Cell Encapsulation Device for Potential Treatment of Type 1 Diabetes,” Proc. Natl. Acad. Sci. USA 115(2):E263-E272 (2017); An et al., “Developing Robust, Hydrogel-based, Nanofiber-enabled Encapsulation Devices (NEEDs) for Cell Therapies,” Biomaterials 37:40-48 (2015), which are hereby incorporated by reference in their entirety), SHIELD allows scale-up not only in the longitudinal but also radial directions without significantly sacrificing the mass transfer or diffusion distance. In principle, a clinically relevant cell loading capacity may be achievable with a reasonable length (i.e. on the order of tens of centimeters instead of meters). Second, SHIELD has a nanofibrous membrane as the primary barrier to prevent cell escape or penetration. Electrospun nanofiber membranes have several unique properties that make them an excellent candidate for cell encapsulation, such as excellent mechanical properties, high porosity, tunable and interconnected pore structures. These properties enable optimization of mass transfer while ensuring safety that is of utmost importance for delivering SC-β cells in clinical applications. Third, SHIELD has a zwitterionically modified alginate hydrogel as the outer skin, mitigating cellular overgrowth for long-term implantation. Importantly, to achieve a thin, uniform, and robust hydrogel coating, we developed an “in-out crosslinking” strategy. Both the superior biocompatibility and stability of the hydrogel coating were critical to supporting the long-term function of insulin-producing cells.

The pore size of the nanofibrous membrane and coating conditions to achieve a SHIELD with balanced safety and functionality have been systemically investigated. The optimized SHIELD significantly reduced cellular growth compared with uncoated devices or those with control alginate coatings. As a result, it was demonstrated that the device could support the long-term function of rat islets in immunocompetent mice for up to 399 days. More importantly, it was found that human SC-β cells at a high density survived in the device and restored normoglycemia of immunodeficient diabetic mice shortly after implantation without any maturation period, for up to 238 days. Lastly, large-animal studies were performed to demonstrate the scalability and retrievability by intraperitoneally implanting 12 cm-long devices in dogs. The devices could be conveniently implanted and rapidly retrieved using minimally invasive laparoscopic procedures. All these results provide a proof of concept for the potential of SHIELD to safely deliver human SC-β cells to T1D patients.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. An implantable therapeutic delivery system comprising:
a nanofiber core substrate having proximal and distal ends, said nanofiber core substrate having an interior nanofiber wall defining an internal space extending longitudinally along the core substrate, with one or more therapeutic agents positioned within the internal space; and
a hydrogel surrounding said nanofiber core substrate, wherein said hydrogel comprises 0.1% to 20% of an alginate mixture, said alginate mixture comprising zwitterionically modified alginate and pure alginate in a ratio of 1:1000 to 1000:1 (v/v).

2. An implantable therapeutic delivery system comprising:
a nanofiber core substrate having proximal and distal ends, said nanofiber core substrate having an interior nanofiber wall defining an internal space extending longitudinally along the core substrate, with one or more therapeutic agents positioned within the internal space; and
a biocompatible polymeric coating surrounding said nanofiber core substrate, wherein said biocompatible polymeric coating has a thickness of 1 nm to 5 mm, and wherein the standard deviation in thickness of the polymeric coating around the entirety of the nanofiber core substrate is <100%.

3. The implantable therapeutic delivery system of claim 2, wherein the standard deviation in thickness of the polymeric coating around the entirety of the nanofiber core substrate is <60%.

4. The implantable therapeutic delivery system of claim 1, wherein the alginate mixture comprises zwitterionically modified alginate and pure alginate in a ratio of 7:3 to 3:7 (v/v).

5. The implantable therapeutic delivery system of claim 1 or claim 2, wherein the interior nanofiber wall of the nanofiber core substrate forms a tube having a diameter of 0.1 mm to 30 cm.

6. The implantable therapeutic delivery system of any one of claims 1-5, wherein the interior wall has a thickness of 1 μm to 5 mm.

7. The implantable therapeutic delivery system of any one of claims 1-6, wherein the nanofiber core substrate has a nanofiber density of 0.01 g/cm³ to 1.5 g/cm³.

8. The implantable therapeutic delivery system of any one of claims 1-7, wherein nanofibers of the nanofiber core substrate have a diameter of 1 nm to 50 μm.

9. The implantable therapeutic delivery system of any one of claims 1-8, wherein the nanofiber core substrate comprises pores, said pores having a diameter of 1 nm to 50 μm.

10. The implantable therapeutic delivery system of any one of claims 1-9, wherein nanofiber composition of the nanofiber core substrate is homogeneous.

11. The implantable therapeutic delivery system of any one of claims 1-9, wherein nanofiber composition of the nanofiber core substrate is heterogeneous.

12. An implantable therapeutic delivery system comprising:
a nanofiber core substrate having proximal and distal ends, said nanofiber core substrate defined by an inner nanofiber layer and an outer nanofiber layer surrounding the inner nanofiber layer, wherein the inner nanofiber layer has a nanofiber structure that differs from the nanofiber structure of the outer nanofiber layer, said nanofiber core substrate further comprising an internal space surrounded by the inner nanofiber layer of the substrate, with one or more therapeutic agents positioned within said internal space; and
a biocompatible polymeric coating surrounding said nanofiber core substrate.

13. The implantable therapeutic delivery system of claim 12, wherein the nanofiber core substrate comprises one or more middle nanofiber layers positioned between the inner and outer nanofiber layers of the substrate, each middle nanofiber layer comprising a nanofiber structure that differs from the nanofiber structure of the inner and outer nanofiber layers.

14. The implantable therapeutic delivery system of claim 12, wherein the nanofiber substrate is a cylindrical tube.

15. The implantable therapeutic delivery system of claim 12, wherein the nanofiber substrate is a conical tube.

16. The implantable therapeutic delivery system of any one of claims 12-15, wherein nanofibers of the inner nanofiber layer and outer nanofiber layer independently have a diameter of 1 nm to 50 μm.

17. The implantable therapeutic delivery system of any one of claims 12-16, wherein the inner nanofiber layer and the outer nanofiber layer independently have a nanofiber density of 0.01 g/cm³ to 1.5 g/cm³.

18. The implantable therapeutic delivery system of any one of claims 12-17, wherein the inner nanofiber layer and the outer nanofiber layer independently have an average thickness of 1 μm to 5 mm.

19. The implantable therapeutic delivery system of any one of claim 12-18, wherein the inner nanofiber layer comprises pores, said pores having a diameter of 1 nm to 50 μm.

20. The implantable therapeutic delivery system of any one of claims 12-19, wherein the outer nanofiber layer comprises pores, said pores having a diameter of 1 nm to 50 μm.

21. The implantable therapeutic delivery system of any one of claims 12-20, wherein the inner and outer nanofiber layers of the core substrate have a combined thickness of 1 μm to 5 mm.

22. The implantable therapeutic delivery system of any one of claims 1-21, wherein the nanofiber core substrate has a length of 0.5 cm to 1000 m.

23. The implantable therapeutic delivery system of any of claims 1-22, wherein the nanofiber core substrate comprises one or more biologically active agents selected from the group consisting of a protein, peptide, antibody or antibody fragment thereof, antibody mimetic, a nucleic acid, a small molecule, a hormone, a growth factor, an angiogenic factor, a cytokine, an anti-inflammatory agent, and combinations thereof.

24. The implantable therapeutic delivery system according to any of claims 1-23, wherein the nanofiber core substrate comprises a material that is insoluble in the one or more biocompatible polymeric coatings surrounding the substrate.

25. The implantable therapeutic delivery system of any one of claims 1-24, wherein the nanofiber core substrate is translucent.

26. The implantable therapeutic delivery system of any one of claims 1-25, wherein an elongated polymeric scaffold is positioned within the internal space of the nanofiber core substrate.

27. The implantable therapeutic delivery system of any one of claims 1-26, wherein said internal space of the nanofiber core substrate is compartmentalized into two or more sub-internal spaces by one or more internal nanofiber walls.

28. The implantable therapeutic delivery system of any one of claims 1-27, wherein the one or more therapeutic agents positioned within the internal space of the nanofiber core substrate is selected from the group consisting of a protein, peptide, antibody or antibody fragment thereof, antibody mimetic, a nucleic acid, a small molecule, a hormone, a growth factor, an angiogenic factor, a cytokine, an anti-inflammatory agent, and combinations thereof.

29. The implantable therapeutic delivery system according to any one of claims 1-28, wherein a preparation of cells is positioned in the internal space of the nanofiber core substrate and the one or more therapeutic agents is released from said preparation of cells.

30. The implantable therapeutic delivery system of any one of claims 1-29, wherein said proximal and distal ends of the nanofiber core substrate are sealed.

31. The implantable therapeutic delivery system of any one of claims 2-30, wherein the outer biocompatible polymeric coating is a hydrogel material.

32. The implantable therapeutic delivery system of any one of claims 2-31, wherein the biocompatible polymeric coating is crosslinked and interlocked to the nanofiber core substrate.

33. The implantable therapeutic delivery system of any one of claims 2-32, wherein the biocompatible polymeric coating comprises one or more biologically active agents selected from the group consisting of a protein, peptide, antibody or antibody fragment thereof, antibody mimetic, a nucleic acid, a small molecule, a hormone, a growth factor, an angiogenic factor, a cytokine, an anti-inflammatory agent, and combinations thereof.

34. The implantable therapeutic delivery system of claim 1, wherein the hydrogel surrounding the nanofiber core substrate is crosslinked and interlocked to the nanofiber core substrate.

35. The implantable therapeutic delivery system of claim 1, wherein the hydrogel surrounding the nanofiber core substrate has a thickness of 1 nm to 5 mm, wherein the standard deviation in thickness of the hydrogel around the entirety of the nanofiber core substrate is <100%.

36. The implantable therapeutic delivery system of claim 1, wherein the hydrogel surrounding the nanofiber core substrate comprises one or more biologically active agents selected from the group consisting of a protein, peptide, antibody or antibody fragment thereof, antibody mimetic, a nucleic acid, a small molecule, a hormone, a growth factor, an angiogenic factor, a cytokine, an anti-inflammatory agent, and combinations thereof.

37. The implantable therapeutic delivery system of claim 12, wherein the biocompatible polymeric coating surrounding the nanofiber core substrate has a thickness of 1 nm to 5 mm, wherein the standard deviation in thickness of the polymeric coating around the entirety of the nanofiber core substrate is <100%.
